SEQUENCE LISTING (14 sequences)

SEQ ID NO: 1 - protein, 631 aa, Aspergillus fumigatus
  misc_feature (1)..(631): AfGA1 precursor
MPRLSYALCALSLGHAAIAAPQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTAAETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWQ

SEQ ID NO: 2 - protein, 631 aa, Aspergillus fumigatus A1163
  misc_feature (1)..(631): AfGA2 precursor
MPRLSYALCALSLGHAAIAAPQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTATETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWR

SEQ ID NO: 3 - protein, 627 aa, Neosartorya fisheri NRRL 181
  misc_feature (1)..(627): Nf_NRRL_181_GA
MPRLSYALCALSLGHAAIAAPQLSPRATGSLDSWLATESTVSLNGILANIGADGAYAKSAKPGIIIASPSTSDPDYYYTWTRDAALVTKVLVDLFRNGNLGLQKVITEYVNSQAYLQTVSTPSGGLSSGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGVAVSAGRYPEDVYYNGNPWFLTTLAAAEQLYDAIYQWKKIGSISITSTSLAFFKDIYSSVAVGTYASSSSTFTAIIDAVKTYADGYVSIVEAHAMTNGSLSEQFDKSSGMSLSARDLTWSYAALLTANMRRNGVVPAPWGAASANSVPSSCSMGSATGTYSTATATSWPSTLTSGSPSDTTSGTTPGTTTTTSACTTPTSVAVTFDEIATTTYGENVYIIGSISQLGSWDTSKAVPLSSSKYTSSNNLWYVTINLPAGTTFEYKYIRKESDGSIEWESDPNRSYTVPSACGVSTATEKDTWR

SEQ ID NO: 4 - protein, 637 aa, Talaromyces stipitatus ATCC 10500
  misc_feature (1)..(637): Ts_ATCC0_10500_GA
MTRLSSVLCALAALGQTALAAPGLSPRASTSLDAWLATETTVSLSGILANIGADGAYSKSAKPGVVIASPSTDNPNYYYTWTRDSALTLKVLIDLFRNGNLGLQTVIEEYVNAQAYLQTVSNPSGDLSSGAGLAEPKFNVDMSAFTGSWGRPQRDGPALRAIALIDFGNWLIENGYTSLAANNIWPIVRNDLSYVAQYWSQSGFDLWEEVNSMSFFTVANQHRSLVEGSTFAAKVGASCSWCDSQAPQILCYMQTFWTGSYMNANTGGGRSGKDANTVLTSIATFDPEATCDDVTFQPCSPRALANHKVYTDSFRSVYGLNSGIAEGVAVAVGRYPEDSYYNGNPWFLSNLAAAEQLYDAIYQWNKIGSITITSTSLAFFKDVYSSAAVGTYASGSSAFTSIINAVKTYADGYISVVQSHAMNNGSLSEQFDKNTGAELSARDLTWSYAALLTANMRRNGVVPPSWGAASATSIPSSCTTGSAIGTYSTPTATSWPSTLTSGTGSPGSTTSATGSVSTSVSATTTSAGSCTTPTSVAVTFDEIATTSYGENVYIVGSISQLGSWNTANAIALSASKYTTSNNLWYVTINLPAGTTFQYKYIRKESDGTVKWESDPNRSYTVPSACGVSTATENDTWR

SEQ ID NO: 5 - protein, 641 aa, Penicillium marneffei ATCC 18224
  misc_feature (1)..(641): Pm_ATCC_18224_GA
MTFSRLSSSVLCALAALGHNALAAPQFSPRATVGLDAWLASETTFSLNGILANIGSSGAYSASAKPGVVIASPSTNNPNYYYTWTRDSALTLKVLIDLFGNGNLSLQTVIEEYINAQAYLQTVSNPSGDLSSGAGLAEPKYNVDMSPFTGGWGRPQRDGPALRAIALIEFGNWLIDNGYSSYAVNNIWPIVRNDLSYVSQYWSQSGFDLWEEVNSMSFFTVANQHRALVQGSTFAARVGASCSWCDSQAPQILCYMQTFWTGSYINANTGGGRSGKDSNTVLTTIHTFDPEATCDDVTFQPCSPRALANHKVYTDSFRSIYGVNSGIAQGVAVSVGRYPEDSYYGGNPWFLSNLAAAEQLYDAIYQWNKIGSITITSTSLAFFKDVYSSAAVGTYASGSTAFTSIISAVKTYADGYVSIVQGHAAANGSLSEQFDRNSGVEISARDLTWSYAALLTANLRRNGVMPPSWGAASANSVPSSCSMGSATGTYSTPTATAWPSTLTSATGIPVTTSATASVTKATSATSTTTSATTCTTPTSVAVTFDEIATTTYGENVFIVGSISQLGSWDTSKAIALSASQYTSSNHLWFATLSLPAGTTFQYKYIRKESNGSIVWESDPNRSYTVPSGCGVSTATENDTWR

SEQ ID NO: 6 - protein, 661 aa, Aspergillus nidulans FGSC A4
  misc_feature (1)..(661): An_FGSC_A4_GA
MPTTILKITLFPLIDSIFSVQLSPVRIAMLTLSKVLPVLALSHAVAAAPQLSARATASLNTWLSTEASFALDGILTNIGANGAYAKTAKAGADYYTWTRDAALTVKVLVDLFHNGDLSLQTILEEYTNSQAYLQTVSNPSGGLASGGLAEPKFYVDMTAFTGSWGRPQRDGPALRATTLIGFGNWLIDNGYSSYASNNIWPIVRNDLTYVAQYWSKSGYDLWEEVNSMSFFTVAVQHRALVEGSTFAHRVGASCPWCDSQAPQILCYMQNFWTGSYINANTGGGRSGKDANTVLASIHTFDPDAACDDITFQPCSSRALANHKVYTDSFRSVYSLNTGIAQGVAVAAGRYPEDSYYNGNPWFLTTLAAAEQLYDAIYQWQKARSISITSTSLAFFKDIYSSAAVGTYASGSSAFTAIIDAVKTYADGYVSIVKAHAMANGSLSEQFDKTYGTCVSARDLTWSYAALLTASMRRNGVVPPSWDAASANTLPSSCSTGSATGTYSTATVTTWPSTLTSGSASATTTIMATSTATSSSTTTSTTTACTTPSTVAVTFNVIATTTYGENVYIVGSISQLGNWDTGSAVALSASKNTSSNNLWYVDINLPGGTAFEYKYIRKETDGSIVWESDPNRSYTVPSSCGVSTATESDTWRCTLETQSVRN

SEQ ID NO: 7 - protein, 97 aa, Aspergillus fumigatus
  misc_feature (1)..(97): AfGA1 and AfGA2 CBM
FNEIATTTYGENVYIVGSISELGNWDTSKAVALSASKYTSSNNLWYVSVTLPAGTTFEYKYIRKESDGSIVWESDPNRSYTVPAACGVSTATENDTW

SEQ ID NO: 8 - DNA, 2060 bp, Aspergillus fumigatus
  misc_feature (1)..(2060): AfGA1 gene of pTrex3gM-AfGA1
atgcctcgcctttcctacgcgctctgtgcgctgtctctcgggcatgctgctattgcagctcctcagttatccgctcgtgctaccggcagcttggactcctggttgggtactgagaccaccgttgcgctcaatggtattctggccaacatcggtgccgacggtgcttatgcgaagagcgctaagcctggcataatcattgccagtccgagcaccagcgaaccagactgtgagaaccttcctgaactggccctgtccggcagtcattgacctcggtagactactatacctggacgagagatgctgctctcgtcacgaaagtcctggtcgacctcttccgcaacggcaacctgggtctgcagaaagtcattaccgaatacgtcaactctcaggcgtacttgcagaccgtgtctaatccgtcgggtggtcttgcgagcggaggtctcgcggagcctaagtacaacgtcgacatgacggcctttaccggagcctggggtcgtcctcagcgtgatggtccggctctgcgggccaccgccctcatcgactttggcaactggctgattgtatgttctccatacgagccccaggaagcgttgctgacgtctacaggacaacggctactccagctatgctgtcaacaacatctggcccattgtgcgcaacgacttgtcctacgtttctcagtactggagccagagtggctttggtgagtcccgactctctggaagtttacaacgtgcatcgattactgacaattgagattctacgtgacagatctctgggaagaagtcaactccatgtccttcttcaccgtcgctgtccagcaccgtgccctcgtggagggaagcacgttcgctaaacgggtgggagcgtcgtgctcgtggtgtgactcgcaggccccccagatcctctgctacatgcagagtttctggactggctcgtatatcaacgccaacaccggtggtggccggtccggcaaggatgccaacaccgtcctcgccagcatccataccttcgaccccgaagccggctgcgacgatactactttccagccctgctctcctcgggcccttgccaaccacaaggtgtacaccgattcgttccgctcggtctacgcgatcaactccggcatcccacagggcgctgccgtttccgctggccgctaccccgaggacgtctactacaacggcaacccttggttcctcaccaccctcgccgctgccgagcagctctacgacgctatctaccagtggaagaagatcggttccatcagcatcaccagcacctccctcgccttcttcaaggacatctacagctccgccgcggtcggcacctacgcctctagcacctccaccttcacggacatcatcaacgcggtcaagacctacgcagacggctacgtgagcatcgtccaggcacacgccatgaacaacggctccctttcggagcaattcgacaagtcctctgggctgtccctctccgcccgcgatctgacctggtcctacgccgctttcctcaccgccaacatgcgtcgtaacggcgtggtgcctgccccctggggcgccgcctccgccaactccgtcccctcgtcttgctccatgggctcggccacgggcacctacagcaccgcgacagccacctcctggcccagcacgctgaccagcggctcgccaggcagcaccaccaccgtgggcaccacgaccagtaccacctctggcaccgccgccgagaccgcctgtgcgacccctaccgccgtggccgtcacctttaacgagatcgccaccaccacctacggcgagaatgtttacattgttgggtccatctccgagctcgggaactgggataccagcaaagcagtggccctgagtgcgtccaagtatacctccagcaataacctctggtacgtgtccgtcaccctgccggctggcacgacattcgagtacaagtatatccgcaaggaaagcgatggctcgatcgtgtgggagagtgaccccaaccgctcgtatacggtgccggcagcttgtggagtgtctactgcgaccgagaatgatacttggcagtga

SEQ ID NO: 9 - DNA, 34 bp, Artificial Sequence
  Synthetic primer AfGA1-Fw
gcggcggccgcaccatgcctcgcctttcctacgc

SEQ ID NO: 10 - DNA, 36 bp, Artificial Sequence
  Synthetic primer AfGA1-Rv
ccggcgcgcccttatcactgccaagtatcattctcg

SEQ ID NO: 11 - protein, 19 aa, Aspergillus fumigatus
  misc_feature (1)..(19): AfGA1 and AfGA2 signal peptide
MPRLSYALCALSLGHAAIA

SEQ ID NO: 12 - protein, 612 aa, Aspergillus fumigatus
  misc_feature (1)..(612): AfGA1 Mature form
APQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTAAETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWQ

SEQ ID NO: 13 - protein, 612 aa, Aspergillus fumigatus
  misc_feature (1)..(612): AfGA2 Mature form
APQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTATETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWR

SEQ ID NO: 14 - DNA, 1896 bp, Aspergillus fumigatus
  misc_feature (1)..(1896): AfGA2 gene of pTrex3gM-AfGA2
atgcctcgactgagctacgctctctgcgctctgtccctgggtcacgctgccatcgccgctccccaactgagcgcccgagctactggcagcctcgattcctggctgggcactgagaccaccgttgctctgaacggcatcctcgctaacatcggcgctgatggtgcctatgccaagagcgctaaacctggcatcatcatcgccagccctagcaccagcgagcctgattactactatacttggacccgcgacgctgctctggtcaccaaggtcctcgttgacctgttccgcaatggtaacctgggcctccagaaagtcattaccgagtacgtcaacagccaagcttatctgcaaaccgttagcaatccctccggtggcctcgcttccggcggcctggccgagcccaaatacaacgtcgacatgaccgcctttaccggtgcctggggtcgcccccagcgagatggccctgccctgcgcgccaccgctctcatcgacttcggcaactggctgatcgacaacggctattccagctatgctgtcaacaacatttggcccatcgtccgcaacgacctgtcctatgtttcccaatactggtcccagtccggtttcgacctctgggaggaggttaattccatgagctttttcaccgtcgctgtccaacatcgagctctcgtcgagggctccactttcgctaagcgcgtcggcgccagctgttcctggtgcgattcccaggcccctcagattctgtgctacatgcagtccttttggaccggtagctatatcaatgccaataccggcggtggtcgaagcggcaaggacgctaatactgttctggcttccatccacaccttcgatcccgaggccggctgtgatgatactacctttcagccctgctcccctcgcgctctcgccaaccataaagtttacaccgacagctttcgcagcgtttacgccatcaactccggcattcctcaaggcgctgctgtttccgctggtcgctaccccgaggacgtttactataatggcaacccctggttcctcactactctggctgctgctgagcagctctatgacgctatctaccaatggaagaaaatcggcagcatcagcattacttccacctccctcgccttcttcaaagacatctatagctccgctgccgttggcacttatgcttcctccactagcactttcactgatattatcaacgctgttaaaacctacgctgacggctacgtcagcatcgttcaagcccacgctatgaacaacggttccctctccgagcagttcgacaagtccagcggtctgagcctcagcgctcgcgacctcacctggtcctacgccgccttcctgactgccaacatgcgccgaaacggcgtcgttcctgccccttggggtgccgccagcgccaattccgtccccagcagctgtagcatgggctccgccactggtacctacagcaccgctaccgctactagctggcccagcaccctgactagcggctcccccggttccactactaccgtcggcaccactacctccaccacttccggtactgccaccgagactgcctgtgccacccctaccgccgtcgccgtcacctttaacgagattgctaccaccacctacggcgagaacgtctacatcgtcggtagcatctccgagctcggcaattgggacacttccaaggctgtcgccctgtccgcctccaaatatactagcagcaacaacctgtggtatgtctccgttaccctgcctgctggtactacttttgagtacaagtacattcgcaaagagtccgatggctccatcgtttgggagtccgatcccaaccgaagctacaccgttcccgctgcttgtggcgtctccactgctactgagaatgacacctggcgctaa

(19) United States
(12) Patent Application Publication
(10) Pub. No.: US 2016/0108448 A1
(43) Pub. Date: Apr. 21, 2016

(54) PROCESS FOR PRODUCING HIGH GLUCOSE COMPOSITIONS BY SIMULTANEOUS LIQUEFACTION AND SACCHARIFICATION OF STARCH SUBSTRATES

(71) Applicant: DANISCO US INC., Palo Alto, CA (US)

(72) Inventors: Jing Ge, Shanghai (CN); Ling Hua, Hockessin, DE (US); Sung Ho Lee, North Liberty, IA (US); Jayarama K. Shetty, Pleasanton, CA (US); Bruce A. Strohm, Beloit, WI (US); Zhongmei Tang, Shanghai (CN); Donald E. Ward, Cedar Rapids, IA (US)

(21) Appl. No.: 14/649,168

(22) PCT Filed: Nov. 21, 2013
(86) PCT No.: PCT/US2013/071162

§ 371 Date: Jun. 2, 2015

(30) Foreign Application Priority Data
Dec. 11, 2012 (CN) PCT/CN2012/086369

(51) Int. Cl. C12P 19/16; C12P 19/02

(52) U.S. Cl. CPC C12P 19/16; C12P 19/02

(57) ABSTRACT

Fungal glucoamylases from Aspergillus fumigatus that are expressed in Trichoderma reesei host cells (AfGATR) are provided. AfGATRs, including AfGA1TR and AfGA2TR, exhibit high activity at elevated temperatures and at low pH, so AfGATRs can be used efficiently in a simultaneous liquefaction and saccharification process in the presence of alpha amylase, such as Aspergillus kawachii alpha-amylase (AkAA). This greatly reduces the combined run time of liquefaction and saccharification reaction, where the pH and temperature must be readjusted for optimal use of the alpha-amylase or glucoamylase.

### CROSS REFERENCE TO RELATED APPLICATIONS

This application claims benefit of priority from international patent application no. PCT/CN2012/086369 filed on 11 Dec. 2012, and is incorporated herein by reference in its entirety.

### SEQUENCE LISTING

A sequence listing comprising SEQ ID NO: 1-14 is attached herein and incorporated by reference in its entirety.

### FIELD OF THE INVENTION

Methods for producing high-glucose compositions by simultaneously saccharifying and liquefying starch using an Aspergillus fumigatus glucoamylase, (AfGATR) or a variant thereof, expressed in Trichoderma reesei host cells.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing α-1,6 branch points every 24-30 glucose units; its MW may be as high as 100 million.

Sugars from starch, in the form of concentrated dextrose syrups, are currently produced by an enzyme catalyzed process involving: (1) liquefaction (or viscosity reduction) of solid starch with an α-amylase into dextrins having an average degree of polymerization of about 7-10, and (2) saccharification of the resulting liquefied starch (i.e. starch hydrolysate) with glucoamylase (also called amyloglucosidase or GA). The resulting syrup has a high glucose content. Much of the glucose syrup that is commercially produced is subsequently enzymatically isomerized to a dextrose/fructose mixture known as isosyrup. The resulting syrup also may be fermented with microorganisms, such as yeast, to produce commercial products including ethanol, citric acid, lactic acid, succinic acid, itaconic acid, monosodium glutamate, gluconates, lysine, other organic acids, other amino acids, and other biochemicals, for example. However, traditional dry milling processes require separate liquefaction and saccharification/fermentation steps, due to differences in the operating temperature of the amylases and glucoamylases used in the process. Simultaneous liquefaction and saccharification (SLS) processes need to be developed to achieve greater economy and efficiency.

α-Amylases, hydrolyze starch, glycogen, and related polysaccharides by cleaving internal α-1,4-glucosidic bonds at random. α-Amylases are often used in high temperature liquefaction processes. Glucoamylases (glucan 1,4-α-glucohydrolases, EC 3.2.1.3), on the other hand, are starch hydrolyzing exo-acting carbohydrases, which catalyze the removal of successive glucose units from the non-reducing ends of starch or related oligo and polysaccharide molecules. Glucoamylases can hydrolyze both the linear and branched glucosidic linkages of starch (e.g., amylose and amylopectin). Glucoamylases are often used to further saccharify starch after the starch has been liquefied. Glucoamylases do not function at high temperatures required for liquefaction. Thus, there is a need for glucoamylases that function at high temperatures for SLS.

Glucoamylases are produced by numerous strains of bacteria, fungi, and plants. For example, a glucoamylase is produced by strains of Aspergillus fumigatus Luo et al. (2008) “Production of acid proof raw starch-digesting glucoamylase from a newly isolated strain of Aspergillus fumigatus MS-09,” Sci. Tech. Food Indus. 29(5): 151-154; Sellars et al. (1976) “Degradation of barley by Aspergillus fumigatus Fres,” Proc. Int. Biodegradation Symp., 3rd, S. J. Miles et al., eds., Appl. Sci., Barking, UK, pp. 635-43; Domingues et al. (1993) “Production of amylase by soil fungi and partial biochemical characterization of amylase of a selected strain (Aspergillus fumigatus Fresenius),” Can. J. Microbiol. 39(7): 681-85; Cherry et al. (2004) “Extracellular glucoamylase from the isolate Aspergillus fumigatus,” Pakistan J. Biol. Sci. 7(11): 1988-92. However, Aspergillus fumigatus is highly allergenic and pathogenic to humans and plants. Thus, Aspergillus fumigatus is not a viable production host for glucoamylases used in industrial processes for manufacturing products for human consumption. There is a need to produce A. fumigatus glucoamylases from a suitable host.

### SUMMARY

The invention relates to a one-step enzymatic conversion process, simultaneous liquefaction and saccharification (SLS), to produce high glucose syrup from gelatinized starch using a liquefying alpha-amylase and a saccharifying glucoamylase above the starch gelatinization temperature. Starch concurrently undergoes gelatinization, liquefaction, and saccharification during simultaneous liquefaction and saccharification in the disclosed methods. In contrast, granular starch still maintains crystalline structure during incubation in a “no-cook” or a direct-starch-to-glucose (DSTG) process. Therefore, due to immediate gelatinization of starch at its operating temperature, SLS significantly reduces the required time for starch solubilization compared to DSTG.

Accordingly, provided is a method of simultaneously liquefying and saccharifying a composition comprising starch to produce a composition comprising glucose, wherein said method comprises: (i) contacting said composition comprising starch with an isolated AfGATR, or variant thereof, having at least 80% sequence identity to SEQ ID NO: 12 or 13 and an α-amylase; and (ii) liquefying and saccharifying the composition comprising starch above the gelatinization temperature of said starch in the same reaction vessel to produce said glucose composition; wherein said AfGATR, or variant thereof, and said α-amylase catalyze the saccharification of the composition comprising starch to the composition comprising glucose.

The AfGATR, or variant thereof, may have at least 70% activity at 74° C. at pH 5.0 over 10 min. The AfGATR, or variant thereof, may be AfGA1TR, or a variant thereof. The AfGA1TR, or variant thereof, may have at least 70% activity over a temperature range of 55°-74° C. at pH 5.0 over 10 min. The AfGA1TR, or variant thereof, may have an optimum temperature of about 68° C. The AfGATR, or variant thereof, may be AfGA2TR, or a variant thereof. The AfGA2TR, or variant thereof, may have at least 70% activity over a temperature range of 61°-74° C. at pH 5.0 over 10 min. The AfGA2TR, or variant thereof, may have an optimum temperature of about 69° C. The AfGATR, or variant thereof, may comprise an amino acid sequence with at least 90%, 95%, or 99% amino acid sequence identity to SEQ ID NO:12. The AfGATR or variant thereof may comprise SEQ ID NO:12. The AfGATR, or variant thereof, may consist of an amino acid sequence with at least 80%, 90%, 95%, or 99% amino acid sequence identity to SEQ ID NO:12. The AfGA1TR, or variant thereof, may consists of SEQ ID NO:12. The AfGATR, or variant thereof, may comprise an amino acid sequence with at least 90%, 95%, or 99% amino acid sequence identity to SEQ ID NO:13. The AfGATR, or variant thereof, may comprise SEQ ID NO:13. The AfGATR, or variant thereof, may consist of an amino acid sequence with at least 80%, 90%, 95%, or 99% amino acid sequence identity to SEQ ID NO:13. The AfGA1TR, or variant thereof, may consist of SEQ ID NO:13.

It is also provided that at least 93% of the starch may be solubilized after about 23 hrs using the disclosed methods. The disclosed composition comprising glucose may comprise at least 90% glucose. The α-amylase may be derived from Bacillus. The α-amylase may be derived from Bacillus stearothermophilus. The disclosed methods may further involve adding a pullulanase.

It is also provided that the starch composition may comprise granular starch or milled starch. The starch composition may comprise a starch from a source selected from the group consisting of corn, wheat, oats, barley, milo, potato, cassava, rye, and rice. The starch composition may comprise a starch from a source selected from the group consisting of corn and wheat.

Liquefaction and saccharification may be conducted at a temperature above the gelatinization temperature of the starch, where the glucoamylase is active at the temperature wherein liquefaction and saccharification is conducted. Liquefaction and saccharification may be conducted between about 60° C. to about 80° C. More specifically, liquefaction and saccharification may be conducted between about 60° C. to about 75° C. More specifically, liquefaction and saccharification may be conducted between about 65° C. to about 75° C. Liquefaction and saccharification may also be conducted between about 70° C. to about 80° C. More specifically, liquefaction and saccharification may be conducted at about 70° C. Liquefaction and saccharification may also be conducted at about 75° C. Liquefaction and saccharification may also be conducted at about 80° C.

Liquefaction and saccharification may be conducted over a pH range of pH 3.0-pH 7.5. More specifically, the pH range may be pH 3.5-pH 7.0. More specifically, the pH range may be pH 4.0-pH 6.7. Liquefaction and saccharification may be conducted for at least 23 hours, at about pH 4.0-6.7 and 34% DS, and at a temperature of about 60° C. to 84° C. Liquefaction and saccharification may be conducted for at least about 23 hours, at about pH 5.0 and at a temperature of about 70° C. to about 80° C.

It is also provided that the disclosed methods may further comprise fermenting the glucose composition to produce an End of Fermentation (EOF) product. The EOF product may comprise a metabolite. The metabolite may be citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, an amino acid, lysine, itaconic acid, 1,3-propanediol, or isoprene.

It is also provided that the disclosed methods may further comprise adding a glucoamylase that is not AfGATR, or variant thereof, hexinase, xylase, glucose isomerase, xylose isomerase, phosphatase, phytase, pullulanase, β-amylase, an additional α-amylase, protease, cellulase, hemicellulase, lipase, cutinase, isoamylase, redox enzyme, esterase, transferase, pectinase, alpha-glucosidase, beta-glucosidase, or a combination thereof, to said starch solution.

The isolated AfGATR, or a variant thereof, may be secreted by a Trichoderma reesei host cell. The host cell may further express and secrete an α-amylase. The host cell may further express and secrete a pullulanase.

Also contemplated is a composition comprising glucose produced by the disclosed methods. Also contemplated is a liquefied starch produced by the disclosed methods. Also contemplated is a fermented beverage produced by the disclosed methods. Also contemplated is a composition for the use of saccharifying a composition comprising starch, comprising an isolated AfGATR, or variant thereof. The AfGATR, or variant thereof, may be purified. The AfGATR, or variant thereof, may be secreted by the host cell.

Also contemplated is the use of the AfGATR, or variant thereof, in the production of a composition comprising glucose. Also contemplated is the use of the AfGATR, or variant thereof, in the production of a liquefied starch.

The disclosed fermented beverage or end of fermentation product may be selected from the group consisting of i) a beer selected from the group consisting of full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, and non-alcoholic malt liquor; and ii) cereal or malt beverages selected from the group consisting of fruit flavored malt beverages, liquor flavored malt beverages, and coffee flavored malt beverages.

### BRIEF DESCRIPTION OF THE DRAWINGS

The accompanying drawings are incorporated in and constitute a part of this specification and illustrate various methods and compositions disclosed herein. In the drawings:

FIGS. 1A-B depict a ClustalW alignment of the AfGA1 catalytic core and carbohydrate binding domain (residues 27-476 and 524-631 of SEQ ID NO: 1, respectively or the full length, with the corresponding residues of glucoamylases from: Aspergillus fumigatus A1163 (AfGA2)(residues 27-476 and 524-631 of SEQ ID NO: 2, respectively); Neosartorya fisheri NRRL 181 (residues 28-476 and 520-627 of SEQ ID NO: 3, respectively); Talaromyces stipitatus ATCC 10500 (residues 28-478 and 530-637 of SEQ ID NO: 4, respectively); Penicillium marneffei ATCC 18224 (residues 31-481 and 534-641 of SEQ ID NO: 5, respectively); and Aspergillus nidulans FGSC A4 (residues 55-493 and 544-661 of SEQ ID NO: 6, respectively). Residues designated by an asterisk in FIG. 1 are AfGA1 residues corresponding to conserved residues in SEQ ID NOS: 1-6.

FIG. 2 depicts a map of a pJG222 expression vector comprising a polynucleotide that encodes an AfGA1 polypeptide, pJG222 (Trex3gM-AfGA1).

FIG. 3 depicts the dependence of glucoamylase activity (relative units) on pH. The glucoamylases include (1) wild-type AfGA expressed in Aspergillus fumigatus, (2) AfGA1TR expressed in Trichoderma reesei, and (2) AnGA expressed in Aspergillus niger. Glucoamylase activity was assayed by the release of glucose from soluble starch at 50° C.

FIG. 4 depicts the dependence of glucoamylase activity (relative units) on temperature. The glucoamylases include (1) wild-type AfGA expressed in Aspergillus fumigatus, (2) AfGA1TR expressed in Trichoderma reesei, and (3) and AnGA. Glucoamylase activity was assayed by the release of glucose from soluble starch at pH 5.0.

FIGS. 5A-B depict AfGA1TR and AnGA glucoamylase activity assayed by the release of reducing sugar from 35% dry solid starch at pH 4.5 and 5.0.

FIGS. 6A-B depict the hydrolysis of 35% dry solid starch to DP1 and reversion of DP1 to DP2 by a composition containing AfGATR1, pullulanase and AkAA.

FIGS. 7A-B depict the hydrolysis of 35% dry solid starch to DP1 and reversion of DP1 to DP2 by a composition containing AfGATR1, pullulanase and varying doses of AkAA.

FIG. 8 depicts the amount of DP2 found in a high glucose composition containing 96% DP1 after the release of reducing sugar from a 35% dry solid starch by compositions containing AfGA1TR or AnGA, and further containing an alpha-amylase (OPTIMAX L-100) and PU (GC636).

FIG. 9 depicts a map of a pJG313 expression vector comprising a polynucleotide that encodes an AfGA2 polypeptide, pJG313 (Trex3gM-AfGA2).

FIG. 10 depicts the dependence of glucoamylase (relative units) of AfGA2TR expressed in Trichoderma reesei on pH. Glucoamylase activity was assayed by the release of glucose from soluble starch substrate at 50° C.

FIG. 11 depicts the dependence of glucoamylase activity (relative units) of AfGA2TR expressed in Trichoderma reesei on temperature. Glucoamylase activity was assayed by the release of glucose from soluble starch substrate at pH 5.0.

FIG. 12 depicts the thermostability of AfGA2TR in 50 mM sodium acetate buffer at pH 5.0. The enzyme was incubated at desired temperature for 2 hours in a thermocycler prior to addition into soluble starch substrate.

FIG. 13 depicts an SDS gel of AfGA1TR expressed in T. reesei. Column M contains a protein molecular weight (MW) ladder in kDa. Columns 1-4 represent samples from T. reesei fermentation producing AfGATR with elapsed fermentation times of 40.5 hours, 64.5 hours, 88.3 hours and 112 hours, respectively. The bands labeled with an arrow at 75 kDa are AfGA1TR.

### DETAILED DESCRIPTION

Fungal glucoamylases from Aspergillus fumigatus (AfGA1TR or AfGA2TR) and variants thereof are provided. AfGA1TR or a variant thereof has a pH optimum of pH 5.0 and at least 70% activity over a range of pH 3.5 to pH 7.5. The enzyme has an optimum temperature of 68° C. and at least 70% activity over a temperature range of 55°-74° C., when tested at pH 5.0. AfGA2TR or a variant thereof has a pH optimum of pH 5.3 and at least 70% activity over a range of pH 3.3 to pH 7.3. The enzyme has an optimum temperature of 69° C. and at least 70% activity over a temperature range of 61°-74° C., when tested at pH 5.0. These properties allow these enzymes to be used in combination with a α-amylase under the same reaction conditions. This obviates the necessity of running a saccharification reaction as a batch process, where the pH and temperature must be adjusted for optimal use of the α-amylase or glucoamylase.

Exemplary applications for AfGATRs (including AfGA1TR and AfGA2TR) or variants thereof can be used in a process of starch saccharification, e.g., SSF, starch liquefaction, e.g., SLS, the preparation of food compositions, the preparation of cleaning compositions, such as detergent compositions for cleaning laundry, dishes, and other surfaces, for textile processing (e.g., desizing). AfGATRs are also statistically significantly more thermostable than AnGA. AfGATRs advantageously catalyze starch saccharification and liquefaction to an oligosaccharide composition significantly enriched in DP1 (i.e., glucose) compared to glucoamylases which do not operate at high temperatures. Furthermore, AfGATRs advantageously catalyze starch saccharification compared to the products of saccharification catalyzed by Aspergillus niger glucoamylase (AnGA). AfGATRs demonstrate a greater rate of saccharification over AnGA, producing more than 96% glucose in 24 hours. AfGATRs can also be used at a lower dosage than AnGA to produce comparable levels of DP1. At least a 50% dose saving can be expected. AfGATRs can be used in combination with enzymes derived from plants (e.g., cereals and grains). AfGATRs also can be used in combination with enzymes secreted by, or endogenous to, a host cell such as T. reesei. For example, AfGATRs can be added to a liquefaction, saccharification, fermentation, SLS or SSF process during which one or more amylases, glucoamylases, proteases, lipases, phytases, esterases, redox enzymes, transferases, or other enzymes from the production host. AfGATRs may be combined with an accessory alpha-amylase to further improve the rate of liquefaction, saccharification or SLS. 0.1 SSU/gds of AkAA also improves the rate of saccharification. When combined with AkAA and a pullulanase, AfGATRs were found to have lower DP3 by 0.1% than AnGA at the same glucose yield in a single pH saccharification process. AfGATRs may also work in combination with endogenous non-secreted production host enzymes. The AfGATRs may also be effective in direct hydrolysis of starch for syrup and/or biochemicals (e.g., alcohols, organic acids, amino acids, other biochemicals and biomaterials) where the reaction temperature is below the gelatinization temperature of substrate.

1. Definitions & Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

Unless defined otherwise, all technical and scientific terms used herein have the same meaning as commonly understood by one of ordinary skill in the art. The following terms are provided below.

1.1. Abbreviations and Acronyms

1. Definitions & Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

Unless defined otherwise, all technical and scientific terms used herein have the same meaning as commonly understood by one of ordinary skill in the art. The following terms are provided below.

1.1. Abbreviations and Acronyms

The following abbreviations/acronyms have the following meanings unless otherwise specified:

ABTS 2,2-azino-bis-3-ethylbenzothiazoline-6-sulfonic acid

AcAmy1 Aspergillus clavatus α-amylase

AE alcohol ethoxylate

AEO alcohol ethoxylate

AEOS alcohol ethoxysulfate

AES alcohol ethoxysulfate

AfGA Aspergillus fumigatus glucoamylase

AfGA1 Aspergillus fumigatus glucoamylase 1

AfGA2 Aspergillus fumigatus glucoamylase 2

AfGATR Aspergillus fumigatus glucoamylase expressed in Trichoderma reesei

AfGA1TR Aspergillus fumigatus glucoamylase 1 expressed in Trichoderma reesei

AfGA2TR Aspergillus fumigatus glucoamylase 2 expressed in Trichoderma reesei

AkAA Aspergillus kawachii α-amylase

AnGA Aspergillus niger glucoamylase

AOS α-olefinsulfonate

AS alkyl sulfate

cDNA complementary DNA

CMC carboxymethylcellulose

DE dextrose equivalent

DNA deoxyribonucleic acid

DPn degree of saccharide polymerization having n subunits

ds or DS dry solids

DTMPA diethylenetriaminepentaacetic acid

EC Enzyme Commission

EDTA ethylenediaminetetraacetic acid

EO ethylene oxide (polymer fragment)

EOF End of Fermentation

FGSC Fungal Genetics Stock Center

GA glucoamylase

GAU/g ds glucoamylase activity unit/gram dry solids

HFCS high fructose corn syrup

HgGA Humicola grisea glucoamylase

HS higher sugar

IPTG isopropyl β-D-thiogalactoside

IRS insoluble residual starch

kDa kiloDalton

LAS linear alkylbenzenesulfonate

MW molecular weight

MWU modified Wohlgemuth unit; 1.6×10⁻⁵ mg/MWU=unit of activity

NCBI National Center for Biotechnology Information

NOBS nonanoyloxybenzenesulfonate

NTA nitriloacetic acid

OxAm Purastar HPAM 5000 L (Danisco US Inc.)

PAHBAH p-hydroxybenzoic acid hydrazide

PEG polyethyleneglycol

pI isoelectric point

ppm parts per million

PVA poly(vinyl alcohol)

PVP poly(vinylpyrrolidone)

RNA ribonucleic acid

SAS alkanesulfonate

SDS-PAGE sodium dodecyl sulfate polyacrylamide gel electrophoresis

SSF simultaneous saccharification and fermentation

SLS simultaneous liquefaction and saccharification

SSU/g solid soluble starch unit/gram dry solids

sp. species

TAED tetraacetylethylenediamine

TrGA Trichoderma reesei glucoamylase

w/v weight/volume

w/w weight/weight

v/v volume/volume

wt % weight percent

° C. degrees Centigrade

H₂O water

dH₂O or DI deionized water

dIH₂O deionized water, Milli-Q filtration

g or gm grams

μg micrograms

mg milligrams

kg kilograms

μL and μl microliters

mL and ml milliliters

mm millimeters

μm micrometer

M molar

mM millimolar

μM micromolar

U units

sec seconds

min(s) minute/minutes

hr(s) hour/hours

DO dissolved oxygen

Ncm Newton centimeter

ETOH ethanol

eq. equivalents

N normal

1.2. Definitions

The terms “amylase” or “amylolytic enzyme” refer to an enzyme that is, among other things, capable of catalyzing the degradation of starch. α-Amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) O-glycosidic linkages within the starch molecule in a random fashion yielding polysaccharides containing three or more (1-4)-α-linked D-glucose units. In contrast, the exo-acting amylolytic enzymes, such as β-amylases (EC 3.2.1.2; α-D-(1→4)-glucan maltohydrolase) and some product-specific amylases like maltogenic α-amylase (EC 3.2.1.133) cleave the polysaccharide molecule from the non-reducing end of the substrate. β-amylases, α-glucosidases (EC 3.2.1.20; α-D-glucoside glucohydrolase), glucoamylase (EC 3.2.1.3; α-D-(1→4)-glucan glucohydrolase), and product-specific amylases like the maltotetraosidases (EC 3.2.1.60) and the maltohexaosidases (EC 3.2.1.98) can produce malto-oligosaccharides of a specific length or enriched syrups of specific malto-oligosaccharides.

As used herein, the term “glucoamylase” (EC 3.2.1.3) (otherwise known as glucan 1,4-α-glucosidase; glucoamylase; amyloglucosidase; γ-amylase; lysosomal α-glucosidase; acid maltase; exo-1,4-α-glucosidase; glucose amylase; γ-1,4-glucan glucohydrolase; acid maltase; 1,4-α-D-glucan glucohydrolase; or 4-α-D-glucan glucohydrolase) refers to a class of enzymes that catalyze the release of D-glucose from the non-reducing ends of starch and related oligo- and polysaccharides. These are exo-acting enzymes, which release glucosyl residues from the non-reducing ends of amylose and amylopectin molecules. The enzymes also hydrolyze alpha-1, 6 and alpha-1, 3 linkages although at much slower rates than alpha-1, 4 linkages. The term “hydrolysis of starch” refers to the cleavage of glucosidic bonds with the addition of water molecules.

The term “pullulanase” (E.C. 3.2.1.41, pullulan 6-glucanohydrolase) refers to a class of enzymes that are capable of hydrolyzing alpha 1-6 glucosidic linkages in an amylopectin molecule.

“Enzyme units” herein refer to the amount of product formed per time under the specified conditions of the assay. For example, a “glucoamylase activity unit” (GAU) is defined as the amount of enzyme that produces 1 g of glucose per hour from soluble starch substrate (4% DS) at 60° C., pH 4.2. A “soluble starch unit” (SSU) is the amount of enzyme that produces 1 mg of glucose per minute from soluble starch substrate (4% DS) at pH 4.5, 50° C. DS refers to “dry solids.”

As used herein “dry solids” or “dissolved solids” content refers to the total solids of a slurry in a dry weight percent basis. The term “slurry” refers to an aqueous mixture containing insoluble solids. The term “high ds” refers to an aqueous starch slurry containing dry solids greater than 38%.

The term “Brix” refers to a well-known hydrometer scale for measuring the sugar content of a solution at a given temperature. The Brix scale measures the number of grams of sucrose present per 100 grams of aqueous sugar solution (the total solubilized solid content). Brix measurements are frequently made by use of a hydrometer or refractometer.

The term “degree of polymerization” (DP) refers to the number (n) of anhydro-glucopyranose units in a given saccharide. Examples of DP1 are monosaccharides, such as glucose and fructose. Examples of DP2 are disaccharides, such as maltose and sucrose. HS or DP4+ (>DP3) denotes polymers with a degree of polymerization of greater than 3. The term “DE,” or “dextrose equivalent,” is defined as the percentage of reducing sugar, i.e., D-glucose, as a fraction of total carbohydrate in a syrup. It is an industry standard for the concentration of total reducing sugars, and is expressed as % D-glucose on a dry weight basis. Unhydrolyzed granular starch has a DE that is essentially 0 and D-glucose has a DE of 100.

As used herein the term “starch” refers to any material comprised of the complex polysaccharide carbohydrates of plants, comprised of amylose and amylopectin with the formula (C₆H₁₀O₅)ₓ, wherein X can be any number. The term includes plant-based materials such as grains, grasses, tubers and roots, and more specifically materials obtained from wheat, barley, corn, rye, rice, sorghum, brans, cassava, millet, potato, sweet potato, and tapioca. The term “starch” includes granular starch. The term “granular starch” refers to raw, i.e., uncooked starch, e.g., starch that has not been subject to gelatinization.

The term “refined starch” refers to purified starch from grains/cereals/tubers.

The term “granular starch” refers to uncooked (raw) starch, which has not been subjected to gelatinization.

The term “starch gelatinization” means solubilization of a starch molecule to form a viscous suspension.

The term “gelatinization temperature” refers to the lowest temperature at which gelatinization of a starch substrate begins. The exact temperature depends upon the specific starch substrate and further may depend on the particular variety of plant species from which the starch is obtained and the growth conditions. Gelatinization temperatures of representative starches are shown in Table 1 below.

The term “starch-liquefying enzyme” refers to an enzyme that affects the hydrolysis or breakdown of granular starch. Exemplary starch liquefying enzymes include alpha amylases (E.C. 3.2.1.1).

The term “liquefact” refers to starch hydrolysate from a conventional high temperature liquefaction process using thermostable alpha amylase.

As used herein, the term “liquefaction” or “liquefy” means a process by which starch is converted to less viscous and shorter chain dextrins.

Saccharifying of starch substrate producing high glucose by glucoamylase above the starch gelatinization of starch refers to the hydrolysis of liquefying starch substrate above the starch gelatinization temperature.

The term “glucose syrup” refers to an aqueous composition containing glucose solids. Glucose syrup will have a DE of at least 20. The term “DE” or “dextrose equivalent” is an industry standard for measuring the concentration of total reducing sugars, calculated as D-glucose on a dry weight basis. (Unhydrolyzed granular starch has a DE that is essentially 0 and D-glucose can have a DE of 100.) In some embodiments, glucose syrup will not contain more than 21% water and will not contain less than 25% reducing sugar calculated as dextrose. In one embodiment, glucose syrup will include at least 90% D-glucose and in another embodiment glucose syrup will include at least 95% D-glucose. In some embodiments the terms glucose and glucose syrup are used interchangeably.

The term “total sugar content” refers to the total sugar content present in a starch composition.

The term “Refractive Index Dry Substance” (RIDS) is defined as the determination of the refractive index of a starch solution at a known DE at a controlled temperature then converting the RI to dry substance using an appropriate relationship, such as the Critical Data Tables of the Corn Refiners Association.

The term “contacting” refers to the placing of the respective enzymes in sufficiently close proximity to the respective substrate to enable the enzymes to convert the substrate to the end product. Those skilled in the art will recognize that mixing solutions of the enzyme with the respective substrates can effect contacting.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide. Further, as used herein and as will be clear from the context, it will be appreciated that referring to a particular sequence as “wild-type” is not meant to imply that other sequences in the example that are not affixed with the pre-fix “wild-type” aren't wild type as well.

As used herein, the term “comparable” in reference to expression level refers to no more than 20% variance between the samples of interest, unless the context clearly dictates otherwise.

Reference to the wild-type protein is understood to include the mature form of the protein. A “mature” polypeptide means a polypeptide or variant thereof from which a signal sequence is absent. For example, the signal sequence may be cleaved during expression of the polypeptide. The mature AfGA1 or AfGA2 is 612 amino acids in length covering positions 1-612 of SEQ ID NO: 1 and SEQ ID NO: 2 respectively, where positions are counted from the N-terminus. The signal sequence of the wild-type AfGA1 or AfGA2 is 19 amino acids in length and has the sequence set forth in SEQ ID NO: 11. Mature AfGA1, AfGA2 or variant thereof may comprise a signal sequence taken from different proteins. The mature protein can be a fusion protein between the mature polypeptide and a signal sequence polypeptide.

The putative “catalytic core” of AfGA1, AfGA2 or a variant thereof spans residues 41-453 of SEQ ID NO: 1. Amino acid residues 534-630 constitute the putative “carbohydrate binding domain” of AfGA1, AfGA2 or a variant thereof. The “linker” or “linker region” of AfGA1, AfGA2 or a variant thereof spans a region between the “catalytic core” and “carbohydrate binding domain.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context. A “variant” of AfGA1 and a “variant glucoamylase polypeptide” are synonymous herein.

In the case of the present enzymes, such as a glucoamylase, “activity” refers to enzymatic activity, which can be measured as described, herein.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an AfGA1 or variant thereof is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature, e.g., an AfGA1 isolated from an A. fumigatus sp. cell. An “isolated” AfGA1 or variant thereof includes, but is not limited to, a culture broth containing secreted AfGA1 expressed in a heterologous host cell (i.e., a host cell this not A. fumigatus).

As used herein, the term “purified” refers to material (e.g., an isolated polypeptide or polynucleotide) that is in a relatively pure state, e.g., at least about 90% pure, at least about 95% pure, at least about 98% pure, or even at least about 99% pure.

The terms “thermostable” and “thermostability,” with reference to an enzyme, refer to the ability of the enzyme to retain activity after exposure to an elevated temperature. The thermostability of an enzyme, such as an amylase enzyme, is measured by its Tm, at which half the enzyme activity is lost under defined conditions. The Tm may be calculated by measuring residual glucoamylase activity following exposure to (i.e., challenge by) an elevated temperature.

A “pH range,” with reference to an enzyme, refers to the range of pH values under which the enzyme exhibits catalytic activity.

As used herein, the terms “pH stable” and “pH stability,” with reference to an enzyme, relate to the ability of the enzyme to retain activity over a wide range of pH values for a predetermined period of time (e.g., 15 min., 30 min., 1 hour).

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

As used herein, “hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. A nucleic acid encoding a variant glucoamylase may have a Tₘ reduced by 1° C.-3° C. or more compared to a duplex formed between the nucleotide of SEQ ID NO: 8 and its identical complement.

As used herein, a “synthetic” molecule is produced by in vitro chemical or enzymatic synthesis rather than by an organism.

As used herein, the terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection,” “transformation” or “transduction,” as known in the art.

A “host strain” or “host cell” is an organism into which an expression vector, phage, virus, or other DNA construct, including a polynucleotide encoding a polypeptide of interest (e.g., AfGA1 or variant thereof) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

As used herein, the term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

As used herein, “biologically active” refer to a sequence having a specified biological activity, such an enzymatic activity.

As used herein, a “swatch” is a piece of material such as a fabric that has a stain applied thereto. The material can be, for example, fabrics made of cotton, polyester or mixtures of natural and synthetic fibers. The swatch can further be paper, such as filter paper or nitrocellulose, or a piece of a hard material such as ceramic, metal, or glass. For amylases, the stain is starch based, but can include blood, milk, ink, grass, tea, wine, spinach, gravy, chocolate, egg, cheese, clay, pigment, oil, or mixtures of these compounds.

As used herein, a “smaller swatch” is a section of the swatch that has been cut with a single hole punch device, or has been cut with a custom manufactured 96-hole punch device, where the pattern of the multi-hole punch is matched to standard 96-well microtiter plates, or the section has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch can also be made by applying a stain to a small piece of material. For example, the smaller swatch can be a stained piece of fabric ⅝″ or 0.25″ in diameter. The custom manufactured punch is designed in such a manner that it delivers 96 swatches simultaneously to all wells of a 96-well plate. The device allows delivery of more than one swatch per well by simply loading the same 96-well plate multiple times. Multi-hole punch devices can be conceived of to deliver simultaneously swatches to any format plate, including but not limited to 24-well, 48-well, and 96-well plates. In another conceivable method, the soiled test platform can be a bead made of metal, plastic, glass, ceramic, or another suitable material that is coated with the soil substrate. The one or more coated beads are then placed into wells of 96-, 48-, or 24-well plates or larger formats, containing suitable buffer and enzyme.

As used herein, “a cultured cell material comprising an AfGA1,” or similar language, refers to a cell lysate or supernatant (including media) that includes an AfGA1 or a variant thereof as a component. The cell material may be from a heterologous host that is grown in culture for the purpose of producing the AfGA1 or variant thereof.

“Percent sequence identity” means that a variant has at least a certain percentage of amino acid residues identical to a wild-type AfGA1, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF. |

Deletions are counted as non-identical residues, compared to a reference sequence. Deletions occurring at either terminus are included. For example, a variant with six amino acid deletions of the C-terminus of the mature AfGA1 polypeptide of SEQ ID NO: 12 would have a percent sequence identity of 99% (606/612 identical residues×100, rounded to the nearest whole number) relative to the mature polypeptide. Such a variant would be encompassed by a variant having “at least 99% sequence identity” to a mature AfGA1 polypeptide.

“Fused” polypeptide sequences are connected, i.e., operably linked, via a peptide bond between the two polypeptide sequences.

The term “filamentous fungi” refers to all filamentous forms of the subdivision Eumycotina.

The phrase “simultaneous liquefaction and saccharification (SLS)” refers to the contemporaneous gelatinization and liquefaction of starch substrates and further saccharification of the starch substrates to saccharides, including glucose.

The phrase “simultaneous saccharification and fermentation (SSF)” refers to a process in the production of biochemicals in which a microbial organism, such as an ethanologenic microorganism, and at least one enzyme, such as AfGA1 or a variant thereof, are present during saccharification and fermentation. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

The phrase “reaction vessel” refers to a container that houses the various enzymes and substrates required for a reaction.

As used herein “ethanologenic microorganism” refers to a microorganism with the ability to convert a sugar or oligosaccharide to ethanol.

The term “fermentation broth” refers to fermentation medium containing the end products after fermentation.

The term “fermented beverage” refers to any beverage produced by a method comprising a fermentation process, such as a microbial fermentation, e.g., a bacterial and/or yeast fermentation.

“Beer” is an example of such a fermented beverage, and the term “beer” is meant to comprise any fermented wort produced by fermentation/brewing of a starch-containing plant material. Often, beer is produced exclusively from malt or adjunct, or any combination of malt and adjunct. Examples of beers include: full malted beer, beer brewed under the “Reinheitsgebot,” ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavored malt beverages, e.g., citrus flavored, such as lemon-, orange-, lime-, or berry-flavored malt beverages, liquor flavored malt beverages, e.g., vodka-, rum-, or tequila-flavored malt liquor, or coffee flavored malt beverages, such as caffeine-flavored malt liquor, and the like.

The term “malt” refers to any malted cereal grain, such as malted barley or wheat.

The term “adjunct” refers to any starch and/or sugar containing plant material which is not malt, such as barley or wheat malt. Examples of adjuncts include common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like.

The term “mash” refers to an aqueous slurry of any starch and/or sugar containing plant material, such as grist, e.g., comprising crushed barley malt, crushed barley, and/or other adjunct or a combination thereof, mixed with water later to be separated into wort and spent grains.

The term “wort” refers to the unfermented liquor run-off following extracting the grist during mashing.

“Iodine-positive starch” or “IPS” refers to (1) amylose that is not hydrolyzed after liquefaction and saccharification, or (2) a retrograded starch polymer. When saccharified starch or saccharide liquor is tested with iodine, the high DPn amylose or the retrograded starch polymer binds iodine and produces a characteristic blue color. The saccharide liquor is thus termed “iodine-positive saccharide,” “blue saccharide,” or “blue sac.”

The terms “retrograded starch” or “starch retrogradation” refer to changes that occur spontaneously in a starch paste or gel on ageing.

The term “about” refers to ±15% to the referenced value.

2. Aspergillus fumigatus Glucoamylases (AfGA1 and AfGA2)

An isolated and/or purified AfGA1, or a variant thereof, polypeptide from A. fumigatus sp., which has glucoamylase activity is provided. The glucoamylase consists of three distinct structural domains, including a catalytic domain, followed by a linker region, that are in turn connected to a starch binding domain. The AfGA1 polypeptide can be the mature AfGA1 polypeptide depicted in SEQ ID NO: 12. The polypeptides may be fused to additional amino acid sequences at the N-terminus and/or C-terminus. Additional N-terminal sequences can be a signal peptide, which may have the sequence shown in SEQ ID NO:11, for example. Other amino acid sequences fused at either termini include fusion partner polypeptides useful for labeling or purifying the protein.

For example, the AfGA1 precursor includes the sequence below (SEQ ID NO:1)

MPRLSYALCALSLGHAAIAAPQLSARATGSLDSWLGTETTVALNGILANI
GADGAYAKSAKPGIIIASPSTSEPDYYYTWTRDAALVTKVLVDLFRNGNL
GLQKVITEYVNSQAYLQTVSNPSGGLASGGLAEPKYNVDMTAFTGAWGRP
QRDGPALRATALIDFGNWLIDNGYSSYAVNNIWPIVRNDLSYVSQYWSQS
GFDLWEEVNSMSFFTVAVQHRALVEGSTFAKRVGASCSWCDSQAPQILCY
MQSFWTGSYINANTGGGRSGKDANTVLASIHTFDPEAGCDDTTFQPCSPR
ALANHKVYTDSFRSVYAINSGIPQGAAVSAGRYPEDVYYNGNPWFLTTLA
AAEQLYDAIYQWKKIGSISITSTSLAFFKDIYSSAAVGTYASSTSTFTDI
INAVKTYADGYVSIVQAHAMNNGSLSEQFDKSSGLSLSARDLTWSYAAFL
TANMRRNGVVPAPWGAASANSVPSSCSMGSATGTYSTATATSWPSTLTSG
SPGSTTTVGTTTSTTSGTAAETACATPTAVAVTFNEIATTTYGENVYIVG
SISELGNWDTSKAVALSASKYTSSNNLWYVSVTLPAGTTFEYKYIRKESD
GSIVWESDPNRSYTVPAACGVSTATENDTWQ

An isolated and/or purified AfGA2, or a variant thereof, polypeptide from A. fumigatus sp., which has glucoamylase activity is also provided. The glucoamylase consists of three distinct structural domains, including a catalytic domain, followed by a linker region, that are in turn connected to a starch binding domain. The AfGA2 polypeptide can be the mature AfGA2 polypeptide depicted in SEQ ID NO: 13. The polypeptides may be fused to additional amino acid sequences at the N-terminus and/or C-terminus. Additional N-terminal sequences can be a signal peptide, which may have the sequence shown in SEQ ID NO:11, for example. Other amino acid sequences fused at either termini include fusion protein polypeptides useful for labeling or purifying the protein.

For example, the AfGA2 precursor includes the sequence below (SEQ ID NO: 2)

MPRLSYALCALSLGHAAIAAPQLSARATGSLDSWLGTETTVALNGILANI
GADGAYAKSAKPGIIIASPSTSEPDYYYTWTRDAALVTKVLVDLFRNGNL
GLQKVITEYVNSQAYLQTVSNPSGGLASGGLAEPKYNVDMTAFTGAWGRP
QRDGPALRATALIDFGNWLIDNGYSSYAVNNIWPIVRNDLSYVSQYWSQS
GFDLWEEVNSMSFFTVAVQHRALVEGSTFAKRVGASCSWCDSQAPQILCY
MQSFWTGSYINANTGGGRSGKDANTVLASIHTFDPEAGCDDTTFQPCSPR
ALANHKVYTDSFRSVYAINSGIPQGAAVSAGRYPEDVYYNGNPWFLTTLA
AAEQLYDAIYQWKKIGSISITSTSLAFFKDIYSSAAVGTYASSTSTFTDI
INAVKTYADGYVSIVQAHAMNNGSLSEQFDKSSGLSLSARDLTWSYAAFL
TANMRRNGVVPAPWGAASANSVPSSCSMGSATGTYSTATATSWPSTLTSG
SPGSTTTVGTTTSTTSGTATETACATPTAVAVTFNEIATTTYGENVYIVG
SISELGNWDTSKAVALSASKYTSSNNLWYVSVTLPAGTTFEYKYIRKESD
GSIVWESDPNRSYTVPAACGVSTATENDTWR

The bolded amino acids above constitute a C-terminal putative carbohydrate binding (CBM) domain (SEQ ID NO: 7) for both AfGA1 and AfGA2. A glycosylated linker region connects the N-terminal catalytic core with the CBM domain. The CBM domain in AfGA1 and AfGA2 is conserved with a CBM20 domain found in a large number of starch degrading enzymes, including alpha-amylases, beta-amylases, glucoamylases, and cyclodextrin glucanotransferases. CBM20 folds as an antiparallel beta-barrel structure with two starch-binding sites 1 and 2. These two sites are thought to differ functionally: site 1 may act as the initial starch recognition site, whereas site 2 may be involved in specific recognition of appropriate regions of starch. See Sorimachi et al. (1997) “Solution structure of the granular starch binding domain of Aspergillus niger glucoamylase bound to beta-cyclodextrin,” Structure 5(5): 647-61. Residues in the AfGA1 and AfGA2 CBM domain that are conserved with starch binding sites 1 and 2 indicated in the sequence below by the numbers 1 and 2, respectively:

```
(SEQ ID NO: 7)
FNEIATTTYGENVYIVGSISELGNWDTSKAVALSASKYTSSNNLWYVSVT
      222222 1 1 1111 2 2222 22
LPAGTTFEYKYIRKESDGSIVWESDPNRSYTVPAACGVSTATENDTW.
                                          1
```

A variant AfGA1 or AfGA2 may comprise some or no amino acid residues of the CBM domain of SEQ ID NO: 7. A variant alternatively may comprise a CBM domain with at least 80%, 85%, 90%, 95%, or 98% sequence identity to the CBM domain of SEQ ID NO: 7. A variant may comprise a heterologous or an engineered CBM20 domain.

The AfGA or variant thereof may be expressed in a eukaryotic host cell, e.g., a filamentous fungal cell that allows proper glycosylation of the linker sequence, for example.

A representative polynucleotide encoding AfGA1 is the polynucleotide sequence set forth in SEQ ID NO: 8. A representative polynucleotide encoding AfGA2 is the polynucleotide sequence set forth in SEQ ID NO: 14. (NCBI Reference Sequence NC_007195 the A. fumigatus genome.) The polypeptide sequence, MPRLSYALCALSLGHAAIA (SEQ ID NO: 11), shown in italics in the AfGA1 and AfGA2 precursor sequences above, is an N-terminal signal peptide that is cleaved when the protein is expressed in an appropriate host cell.

The polypeptide sequence of AfGA1 is similar to other fungal glucoamylases, including AfGA2. For example, AfGA1 has the high sequence identity to the following fungal glucoamylases:
99% sequence identity to the glycosyl hydrolase from Aspergillus fumigatus A1163 (SEQ ID NO: 2)(AfGA2);
92% sequence identity to the glycosyl hydrolase from Neosartorya fisheri NRRL 181 (SEQ ID NO: 3); and
82% sequence identity to the putative glucoamylase from Talaromyces stipitatus ATCC 10500 (SEQ ID NO: 4);
81% sequence identity to the putative glucoamylase from Penicillium marneffei ATCC 18224 (SEQ ID NO: 5);
81% sequence identity to the hypothetical glucoamylase from Aspergillus nidulans FGSC A4 (SEQ ID NO: 6);
   Sequence identity was determined by a BLAST alignment, using the precursor form of the AfGA1 of SEQ ID NO: 1 as the query sequence. See Altschul et al. (1990) J. Mol. Biol. 215: 403-410. Sequence identity may also optionally be based on the mature form of the enzyme.

A variant of an AfGA1 polypeptide is provided. The variant can consist of or comprise a polypeptide with at least 80%, at least 90%, at least 95%, at least 98%, or at least 99% amino acid sequence identity to the polypeptide of residues 1-631 of SEQ ID NO: 1, wherein the variant comprises one or more amino acid modifications selected from a substitution, insertion, or deletion of one or more corresponding amino acids in SEQ ID NO: 2-6. A variant of an AfGA2 polypeptide is also provided. The variant can consist of or comprise a polypeptide with at least 80%, at least 90%, at least 95%, at least 98%, or at least 99% amino acid sequence identity to the polypeptide of residues 1-631 of SEQ ID NO: 2, wherein the variant comprises one or more amino acid modifications selected from a substitution, insertion, or deletion of one or more corresponding amino acids in SEQ ID NO: 1 and/or 3-6. For example, a variant consisting of a polypeptide with at least 99% sequence identity to the polypeptide of residues 1-612 of SEQ ID NO: 1 may have one to six amino acid substitutions, insertions, or deletions, compared to the AfGA1 of SEQ ID NO: 1. The insertions or deletions may be may at either termini of the polypeptide, for example. Alternatively, the variant can “comprise” a polypeptide consisting of a polypeptide with at least 80%, at least 90%, at least 95%, at least 98%, or at least 99% amino acid sequence identity to the polypeptide of 1-631 of SEQ ID NO: 1 or 2. In a variant, additional amino acid residues may be fused to either termini of the polypeptide. The variant may be glycosylated, regardless of whether the variant “comprises” or “consists” of a given amino acid sequence.

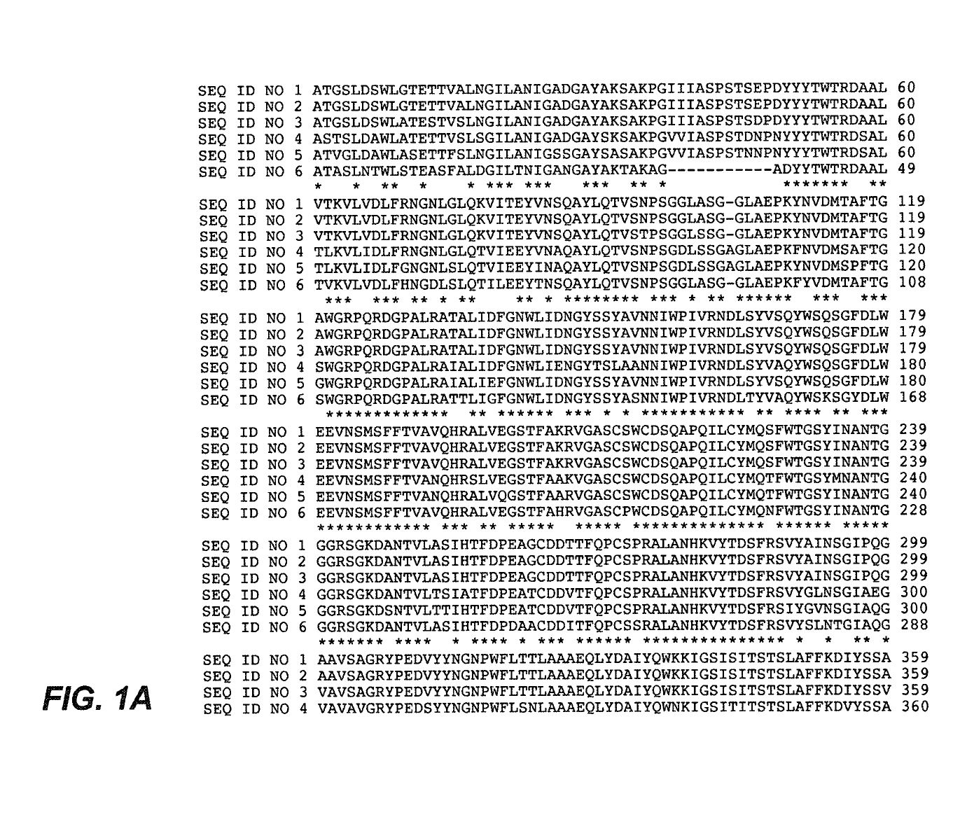

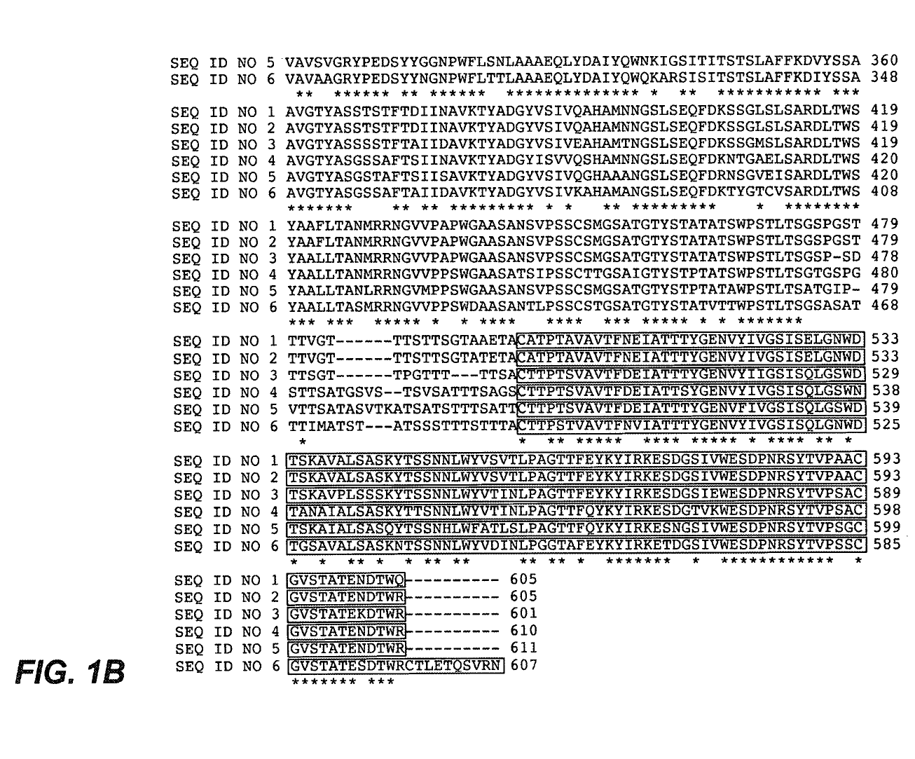

A ClustalW alignment between AfGA1 (SEQ ID NO: 1); AfGA2 (SEQ ID NO: 2); the glucoamylase from Neosartorya fisheri NRRL 181 (SEQ ID NO: 3); the glucoamylase from Talaromyces stipitatus ATCC 10500 (SEQ ID NO: 4); the glucoamylase from Penicillium marneffei ATCC 18224 (SEQ ID NO: 5); and the glucoamylase Aspergillus nidulans FGSC A4 (SEQ ID NO: 6) is shown in FIG. 1. See Thompson et al. (1994) Nucleic Acids Res. 22:4673-4680. As a general rule, the degree to which an amino acid is conserved in an alignment of related protein sequences is proportional to the relative importance of the amino acid position to the function of the protein. That is, amino acids that are common in all related sequences likely play an important functional role and cannot be easily substituted. Likewise, positions that vary between the sequences likely can be substituted with other amino acids or otherwise modified, while maintaining the activity of the protein.

The alignments shown in FIG. 1, for example, can guide the construction of variant AfGA polypeptides having glucoamylase activity. Variants of the AfGA1 polypeptide of SEQ ID NO: 1 can include, but are not limited to, those with an amino acid modification selected from a substitution, insertion, or deletion of a corresponding amino acid in a polypeptide selected from the group consisting of SEQ ID NOS: 2 (AfGA2), 3, 4, 5, and 6. Correspondence between positions in the AfGA1 of SEQ ID NO: 1 and the glucoamylases of SEQ ID NOS: 2, 3, 4, 5 and 6 is determined with reference to the alignment shown in FIG. 1. For example, a variant AfGA1 polypeptide can have the substitution D23N, where Asn is the corresponding amino acid in SEQ ID NO: 6, referring to the alignment in FIG. 1. Variant AfGA1 polypeptides also include, but are not limited to, those with 1, 2, 3, or 4 randomly selected amino acid modifications. Amino acid modifications can be made using well-known methodologies, such as oligo-directed mutagenesis. Similarly, variants of the AfGA2 polypeptide of SEQ ID NO: 2 can include, but are not limited to, those with an amino acid modification selected from a substitution, insertion, or deletion of a corresponding amino acid in a polypeptide selected from the group consisting of SEQ ID NOS: 1 (AfGA1), 3, 4, 5, and 6.

Nucleic acids encoding the AfGA1 polypeptide or variant thereof also are provided. A nucleic acid encoding AfGA1 can be genomic DNA. Or, the nucleic acid can be a cDNA comprising SEQ ID NO: 8. Similarly, nucleic acids encoding the AfGA2 polypeptide or variant thereof also are provided. A nucleic acid encoding AfGA2 can also be genomic DNA. Or, the nucleic acid can be a cDNA comprising SEQ ID NO: 14. As is well understood by one skilled in the art, the genetic code is degenerate, meaning that multiple codons in some cases may encode the same amino acid. Nucleic acids include all genomic DNA, mRNA, and cDNA sequences that encode an AfGA1, AfGA2 or variant thereof.

The AfGA1, AfGA2 or variants thereof may be “precursor,” “immature,” or “full-length,” in which case they include a signal sequence, or “mature,” in which case they lack a signal sequence. The variant glucoamylases may also be truncated at the N- or C-termini, so long as the resulting polypeptides retain glucoamylases activity.

2.1. AfGA Variant Characterization

Variant AfGA polypeptides retain glucoamylase activity. They may have a specific activity higher or lower than the wild-type AfGA polypeptide. Additional characteristics of the AfGA variant include stability, pH range, temperature profile, oxidation stability, and thermostability, for example. For example, the variant may be pH stable for 24-60 hours from pH 3 to about pH 8, e.g., pH 3.0-7.8; e.g., pH 3.0-7.5; pH 3.5-7.0; pH 4.0-6.7; or pH 5.0. An AfGA variant can be expressed at higher levels than the wild-type AfGA, while retaining the performance characteristics of the wild-type AfGA. AfGA variants also may have altered oxidation stability in comparison to the parent glucoamylase. For example, decreased oxidation stability may be advantageous in compositions for starch liquefaction. The variant AfGA, have altered temperature profile compared to the wild-type glucoamylase. Such AfGA variants are advantageous for use in baking or other processes that require elevated temperatures. Levels of expression and enzyme activity can be assessed using standard assays known to the artisan skilled in this field, including those disclosed below.

3. Production of AfGA1 and Variants Thereof

The AfGA or variant thereof can be isolated from a host cell, for example by secretion of the AfGA or variant from the host cell. A cultured cell material comprising AfGA or variant thereof can be obtained following secretion of the AfGA or variant from the host cell. The AfGA or variant is optionally purified prior to use. The AfGA gene can be cloned and expressed according to methods well known in the art. Suitable host cells include bacterial, plant, or yeast cells, e.g., filamentous fungal cells. Particularly useful host cells include Trichoderma reesei. Trichoderma reesei host cells express AfGATRs at higher, or at least comparable, levels to natively expressed AfGA Aspergillus fumigatus.

In some embodiments, the AfGA is heterologously expressed in a host at at least 10 g/liter. In some embodiments, the AfGA is heterologously expressed at at least 15, 20, 25, 30, 35, 40, 45, 50, 55, 60, 65, 70, 75, 80, 90, 100, or 110 g/liter. In some embodiments, the AfGA is heterologously expressed in a Trichoderma reesei host, wherein the expression is at least 15, 20, 25, 30, 35, 40, 45, 50, 55, 60, 65, 70, 75, 80, 90, 100, or 110 g/liter. In some embodiments, the AfGA is heterologously expressed in an Aspergillus host, wherein the expression is at least 15, 20, 25, 30, 35, 40, 45, 50, 55, 60, 65, 70, 75, 80, 90, 100, or 110 g/liter.

The host cell may further express a nucleic acid encoding a homologous or heterologous amylase, i.e., an amylase that is not the same species as the host cell, or one or more other enzymes. The amylase may be a variant amylase. Additionally, the host may express one or more accessory enzymes, proteins, peptides. These may benefit liquefaction, saccharification, SLS, fermentation, SSF, etc. processes. Furthermore, the host cell may produce biochemicals in addition to enzymes used to digest the carbon feedstock(s). Such host cells may be useful for fermentation or simultaneous saccharification and fermentation processes to reduce or eliminate the need to add enzymes.

3.1. Vectors

A DNA construct comprising a nucleic acid encoding an AfGA or variant thereof can be constructed to be expressed in a host cell. Representative nucleic acids that encode AfGA1 include SEQ ID NO: 8. Representative nucleic acids that encode AfGA2 include SEQ ID NO: 14. Because of the well-known degeneracy in the genetic code, variant polynucleotides that encode an identical amino acid sequence can be designed and made with routine skill. It is also well-known in the art to optimize codon use for a particular host cell. Nucleic acids encoding an AfGA or variant thereof can be incorporated into a vector. Vectors can be transferred to a host cell using well-known transformation techniques, such as those disclosed below.

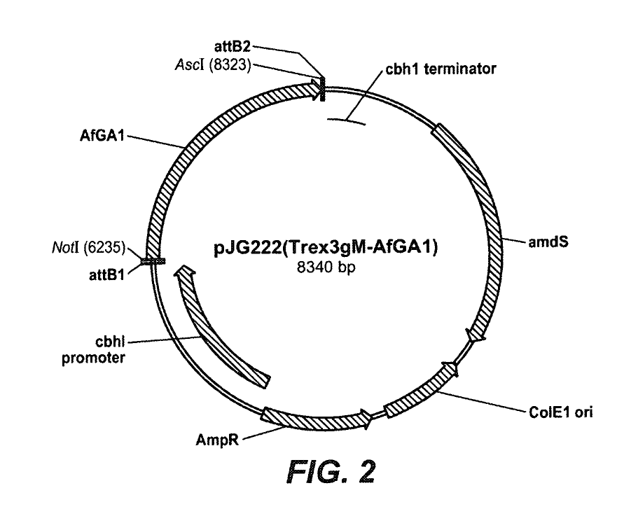

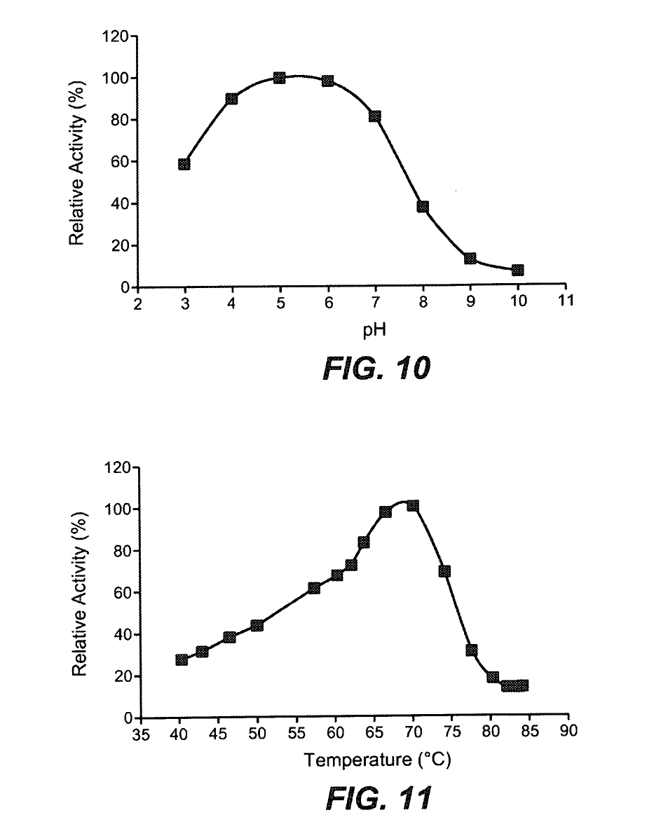

The vector may be any vector that can be transformed into and replicated within a host cell. For example, a vector comprising a nucleic acid encoding an AfGA or variant thereof can be transformed and replicated in a bacterial host cell as a means of propagating and amplifying the vector. The vector also may be transformed into an expression host, so that the encoding nucleic acids can be expressed as a functional AfGA or variant thereof. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). Representative vectors include pJG222 (Trex3gM-AfGA1) (FIG. 2) and pJG313 (Trex3gM-AfGA2) (FIG. 10), each of which comprises a pTrex3gM expression vector (U.S. Published Application No. 2011/0136197 A1), and allows expression a nucleic acid encoding AfGA under the control of the cbh1 promoter in a fungal host. Both pJG222 and pJG313 can be modified with routine skill to comprise and express a nucleic acid encoding an AfGA variant.

A nucleic acid encoding an AfGA or a variant thereof can be operably linked to a suitable promoter, which allows transcription in the host cell. The promoter may be any DNA sequence that shows transcriptional activity in the host cell of choice and may be derived from genes encoding proteins either homologous or heterologous to the host cell. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding an AfGA or variant thereof is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. The pJG222 vector depicted in FIG. 2, for example, contains a cbh1 promoter operably linked to AfGA1. The pJG313 vector depicted in FIG. 10, contains a cbh1 promoter operably linked to AfGA2. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the AfGA gene to be expressed. For example, the DNA may encode the AfGA1 and AfGA2 signal sequence of SEQ ID NO: 11 operably linked to a nucleic acid encoding an AfGA or a variant thereof. The DNA encodes a signal sequence from a species other than A. fumigatus. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding an AfGA or variant thereof. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD, and sC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of an AfGA or variant thereof for subsequent purification. Extracellular secretion of the AfGA or variant thereof into the culture medium can also be used to make a cultured cell material comprising the isolated AfGA or variant thereof.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the AfGA or variant thereof to a host cell organelle such as a peroxisome, or to a particular host cell compartment. Such a targeting sequence includes but is not limited to the sequence serine-lysine-leucine (SKL), which is a known peroxisome target signal. For expression under the direction of control sequences, the nucleic acid sequence of the AfGA or variant thereof is operably linked to the control sequences in proper manner with respect to expression.

The procedures used to ligate the DNA construct encoding an AfGA or variant thereof, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

3.2. Transformation and Culture of Host Cells

A Trichoderma reesei host cell, either comprising a DNA construct or an expression vector, is advantageously used as a host cell in the recombinant production of an AfGATR or variant thereof. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding an AfGA or variant thereof is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

3.3. Expression

A method of producing an AfGATR or variant thereof may comprise cultivating a Trichoderma reesei host cell as described above under conditions conducive to the production of the enzyme and recovering the enzyme from the cells and/or culture medium. Trichoderma reesei host cells express AfGATRs at higher, or at least comparable, levels to natively expressed AfGA Aspergillus fumigatus.

The medium used to cultivate the cells may be any conventional medium suitable for growing the host cell in question and obtaining expression of an AfGATR or variant thereof. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a glucoamylase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the glucoamylase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding AfGA or a variant thereof in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of the AfGATR or variant thereof. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sephorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 78° C. (e.g., 30° C. to 45° C.), depending on the needs of the host and production of the desired AfGATR or variant thereof. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of an AfGATR or variant thereof.

3.4. Identification of AfGATR Activity

To evaluate the expression of an AfGATR or variant thereof in a host cell, assays can measure the expressed protein, corresponding mRNA, or glucoamylase activity. For example, suitable assays include Northern blotting, reverse transcriptase polymerase chain reaction, and in situ hybridization, using an appropriately labeled hybridizing probe. Suitable assays also include measuring AfGATR activity in a sample, for example, by assays directly measuring reducing sugars such as glucose in the culture media. For example, glucose concentration may be determined using glucose reagent kit No. 15-UV (Sigma Chemical Co.) or an instrument, such as Technicon Autoanalyzer. Glucoamylase activity also may be measured by any known method, such as the PAHBAH or ABTS assays, described below.

3.5. Methods for Purifying an AfGATR and Variants Thereof

Fermentation, separation, and concentration techniques are well known in the art and conventional methods can be used in order to prepare a concentrated AfGATR or variant glucoamylase polypeptide-containing solution.

After fermentation, a fermentation broth is obtained, the microbial cells and various suspended solids, including residual raw fermentation materials, are removed by conventional separation techniques in order to obtain an amylase solution. Filtration, centrifugation, microfiltration, rotary vacuum drum filtration, ultrafiltration, centrifugation followed by ultra-filtration, extraction, or chromatography, or the like, are generally used.

It is desirable to concentrate an AfGATR or variant glucoamylase polypeptide-containing solution in order to optimize recovery. Use of unconcentrated solutions can require increased incubation time in order to collect the purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of purification include but are not limited to rotary vacuum filtration and/or ultrafiltration.

The enzyme solution is concentrated into a concentrated enzyme solution until the enzyme activity of the concentrated AfGATR or variant glucoamylase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides, and blends of two or more of these metal halides. Exemplary metal halides include sodium chloride, potassium chloride, sodium bromide, potassium bromide, and blends of two or more of these metal halides. The metal halide precipitation agent, sodium chloride, can also be used as a preservative.

The metal halide precipitation agent is used in an amount effective to precipitate the AfGATR or variant thereof. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific AfGATR or variant glucoamylase polypeptide and on its concentration in the concentrated enzyme solution.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of said organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both amylase preservative agents. For further descriptions, see, e.g., U.S. Pat. No. 5,281,526.

Addition of the organic compound precipitation agent provides the advantage of high flexibility of the precipitation conditions with respect to pH, temperature, AfGATR or variant glucoamylase polypeptide concentration, precipitation agent concentration, and time of incubation.

The organic compound precipitation agent is used in an amount effective to improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be purified. Generally, the pH is adjusted at a level near the isoelectric point of the glucoamylase. The pH can be adjusted at a pH in a range from about 2.5 pH units below the isoelectric point (pI) up to about 2.5 pH units above the isoelectric point.

The incubation time necessary to obtain a purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less than about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further purification of the purified enzyme precipitate can be obtained by washing the precipitate with water. For example, the purified enzyme precipitate is washed with water containing the metal halide precipitation agent, or with water containing the metal halide and the organic compound precipitation agents.

During fermentation, an AfGATR or variant glucoamylase polypeptide accumulates in the culture broth. For the isolation and purification of the desired AfGATR or variant glucoamylase, the culture broth is centrifuged or filtered to eliminate cells, and the resulting cell-free liquid is used for enzyme purification. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme-active fraction. For further purification, a conventional procedure such as ion exchange chromatography may be used.

Purified enzymes are useful for laundry and cleaning applications. For example, they can be used in laundry detergents and spot removers. They can be made into a final product that is either liquid (solution, slurry) or solid (granular, powder).

A more specific example of purification, is described in Sumitani et al. (2000) “New type of starch-binding domain: the direct repeat motif in the C-terminal region of Bacillus sp. 195 glucoamylase contributes to starch binding and raw starch degrading,” Biochem. J. 350: 477-484, and is briefly summarized here. The enzyme obtained from 4 liters of a Streptomyces lividans TK24 culture supernatant was treated with (NH₄)₂SO₄ at 80% saturation. The precipitate was recovered by centrifugation at 10,000×g (20 min. and 4° C.) and re-dissolved in 20 mM Tris/HCl buffer (pH 7.0) containing 5 mM CaCl₂. The solubilized precipitate was then dialyzed against the same buffer. The dialyzed sample was then applied to a Sephacryl S-200 column, which had previously been equilibrated with 20 mM Tris/HCl buffer, (pH 7.0), 5 mM CaCl₂, and eluted at a linear flow rate of 7 mL/hr with the same buffer. Fractions from the column were collected and assessed for activity as judged by enzyme assay and SDS-PAGE. The protein was further purified as follows. A Toyopearl HW55 column (Tosoh Bioscience, Montgomeryville, Pa.; Cat. No. 19812) was equilibrated with 20 mM Tris/HCl buffer (pH 7.0) containing 5 mM CaCl₂ and 1.5 M (NH₄)₂SO₄. The enzyme was eluted with a linear gradient of 1.5 to 0 M (NH₄)₂SO₄ in 20 mM Tris/HCL buffer, pH 7.0 containing 5 mM CaCl₂. The active fractions were collected, and the enzyme precipitated with (NH₄)₂SO₄ at 80% saturation. The precipitate was recovered, re-dissolved, and dialyzed as described above. The dialyzed sample was then applied to a Mono Q HR5/5 column (Amersham Pharmacia; Cat. No. 17-5167-01) previously equilibrated with 20 mM Tris/HCl buffer (pH 7.0) containing 5 mM CaCl₂, at a flow rate of 60 mL/hour. The active fractions are collected and added to a 1.5 M (NH₄)₂SO₄ solution. The active enzyme fractions were re-chromatographed on a Toyopearl HW55 column, as before, to yield a homogeneous enzyme as determined by SDS-PAGE. See Sumitani et al. (2000) Biochem. J. 350: 477-484, for general discussion of the method and variations thereon.

For production scale recovery, an AfGATR or variant glucoamylase polypeptide can be partially purified as generally described above by removing cells via flocculation with polymers. Alternatively, the enzyme can be purified by microfiltration followed by concentration by ultrafiltration using available membranes and equipment. However, for some applications, the enzyme does not need to be purified, and whole broth culture can be lysed and used without further treatment. The enzyme can then be processed, for example, into granules.

4. Compositions and Uses of AfGATR and Variants Thereof

AfGATR and its variants are useful for a variety of industrial applications. For example, AfGATR and its variants are useful in a starch conversion process, particularly in a saccharification process of a starch that has undergone liquefaction. The desired end-product may be any product that may be produced by the enzymatic conversion of the starch substrate. For example, the desired product may be a syrup rich in glucose, which can be used in other processes, such as the preparation of HFCS, or which can be converted into a number of other useful products, such as ascorbic acid intermediates (e.g., gluconate; 2-keto-L-gulonic acid; 5-keto-gluconate; and 2,5-diketogluconate); 1,3-propanediol; aromatic amino acids (e.g., tyrosine, phenylalanine and tryptophan); organic acids (e.g., lactate, pyruvate, succinate, isocitrate, and oxaloacetate); amino acids (e.g., serine and glycine); antibiotics; antimicrobials; enzymes; vitamins; and hormones.

The starch conversion process may be a precursor to, or simultaneous with, a fermentation process designed to produce alcohol for fuel or drinking (i.e., potable alcohol). One skilled in the art is aware of various fermentation conditions that may be used in the production of these end-products. AfGATR and variants thereof also are useful in compositions and methods of food preparation. These various uses of AfGATR and its variants are described in more detail below.

4.1. Preparation of Starch Substrates

Those of general skill in the art are well aware of available methods that may be used to prepare starch substrates for use in the processes disclosed herein. Starch-containing materials useful according to the invention include any starch-containing material. Starch-containing material may be obtained from wheat, corn, rye, sorghum (milo), rice, millet, barley, triticale, cassava (tapioca), potato, sweet potato. The starch content of the grain/cereals and tubers depends on variety, growing conditions and nature of the soil. While not meant to limit the invention in any manner, Table 1 shown below provides a general guide to the level of starch found in common cereal grains and tubers, and their corresponding gelatinization temperatures.

TABLE 1
Starch Content and Gelatinization Temperature

| | Starch Content | Gelatinization |
|---|---|---|
| Raw Material | %, 1 | Temperature, C., 1 and 2 |
| Corn | 60-68 | 62-72 |
| Wheat | 60-65 | 58-64 |
| Oats | 50-53 | 59-61 |
| Barley | 55-65 | 52-59 |
| Milo | 60-65 | 68-77 |
| Potato | | 58-68 |
| Cassava | 25-30 | 59-69 |
| Rye | 60-65 | 57-70 |
| Rice | 70-72 | 68-77 |
| (polished) | | |

The starch from a grain may be ground or whole and includes corn solids, such as kernels, bran and/or cobs. The starch may be highly refined raw starch or feedstock from starch refinery processes. In some embodiments, the entire starch containing material may be used, for example, the whole ground grain or fractionated grain may be used, including corn, wheat, rye, barley, sorghum and combinations thereof. In one embodiment, the starch-containing material may be obtained from fractionated cereal grains including fiber, endosperm and/or germ components. Methods for fractionating plant material, such as corn and wheat, are known in the art. In some embodiments, starch-containing material obtained from different sources may be mixed together to obtain material used in the processes of the invention (e.g. corn and Milo or corn and barley). Specifically contemplated starch substrates are corn starch and wheat starch. Various starches also are commercially available. For example, corn starch is available from Cerestar, Sigma, and Katayama Chemical Industry Co. (Japan); wheat starch is available from Sigma; sweet potato starch is available from Wako Pure Chemical Industry Co. (Japan); and potato starch is available from Nakaari Chemical Pharmaceutical Co. (Japan).

The starch substrate can be a crude starch from milled whole grain, which contains non-starch fractions, e.g., germ residues and fibers. Milling may comprise either wet milling or dry milling or grinding. In wet milling, whole grain is soaked in water or dilute acid to separate the grain into its component parts, e.g., starch, protein, germ, oil, kernel fibers. Wet milling efficiently separates the germ and meal (i.e., starch granules and protein) and is especially suitable for production of syrups. In dry milling or grinding, whole kernels are ground into a fine powder and often processed without fractionating the grain into its component parts. In some cases, oils from the kernels are recovered. Dry ground grain thus will comprise significant amounts of non-starch carbohydrate compounds, in addition to starch. Dry grinding of the starch substrate can be used for production of ethanol and other biochemicals. The starch to be processed may be a highly refined starch quality, for example, at least 90%, at least 95%, at least 97%, or at least 99.5% pure. In some embodiments, the milled grain which is used in the process has a particle size such that more than 50% of the material will pass through a sieve with a 500 micron opening and in some embodiments more than 70% of the material will pass through a sieve with a 500 micron opening (see, for example, WO2004/081193).

The milled starch-containing material is normally screened to a specified sieve size and will be combined with water resulting in aqueous slurry. The slurry will comprise between 15 to 55% ds w/w (e.g., 20 to 50%, 25 to 50%, 25 to 45%, 25 to 40%, and 20 to 35% ds). In some embodiments the recycled thin-stillage (backset) will be in the range of 10 to 70% v/v (e.g., 10 to 60%, 10 to 50%, 10 to 40%, 10 to 30%, 10 to 20%, 20 to 60%, 20 to 50%, 20 to 40% and also 20 to 30%).

4.2. Simultaneous Liquefaction and Saccharification of Starch

In an SLS method, liquefied starch is concurrently saccharified into a syrup rich in lower DP, especially DP1 saccharides, using the AfGATR and variants thereof. Generally liquefaction includes gelatinization of starch simultaneously with or followed by the addition of an α-amylase, although additional liquefaction-inducing enzymes optionally may be added. The exact composition of the products of saccharification depends on the combination of enzymes used, as well as the type of granular starch processed. Advantageously, the syrup obtainable using the provided AfGATR and variants thereof may contain a weight percent of DP1 of the total oligosaccharides in the saccharified starch exceeding about 65%, e.g., 70%, 80%, 85%, 90%, 95%, or 96% after 24 hours.

Gelatinization temperature is referred to as the temperature at which the starch granules absorb water and lose their individual crystalline structure to become a viscous liquid gel. Gelatinization is significant in that it is the preliminary process that renders starch susceptible to the current enzymatic hydrolysis for converting insoluble granular starch into very high glucose or malto dextrins. As it can be seen in Table 1, the gelatinization temperature also depends on the source of the starch.

In some embodiments, the starch substrate prepared as described above is slurried with water. The starch slurry may contain starch as a weight percent of dry solids of about 10-55%, about 20-45%, about 30-45%, about 30-40%, or about 30-35%. α-Amylase (EC 3.2.1.1) may be added to the slurry, with a metering pump, for example. The α-amylase typically used for this application is a thermally stable, bacterial α-amylase, such as a Geobacillus stearothermophilus α-amylase. The α-amylase is generally used, for example, at about 1500 units per kg dry matter of starch. To optimize α-amylase stability and activity, the pH of the slurry typically is adjusted to about pH 5.5-6.5 and about 1 mM of calcium (about 40 ppm free calcium ions) typically is added. Geobacillus stearothermophilus variants or other α-amylases may require different conditions. Bacterial α-amylase remaining in the slurry following liquefaction may be deactivated via a number of methods, including lowering the pH in a subsequent reaction step or by removing calcium from the slurry in cases where the enzyme is dependent upon calcium.

Percent solubilization of granular starch can be determined by sampling from the agitated slurry into two 2.5 ml micro-centrifuge tubes. One tube is spun for ˜4 minutes at 13,000 rpm and the refractive index of the supernatant is determined at 30° C. (RIₛᵤₚ). The total dry substance is determined by adding 1 drop of SPEZYME® FRED from a micro disposable-pipette to the second tube, then boiling the second tube for 10 minutes. The second tube is cooled to 30° C. (RIₜₒₜ) and the amount of dry substance is determined. The dry substance of the supernatant and the whole sample (total) are determined using appropriate DE tables. The table for converting RIₛᵤₚ to DS is the 95 DE, Table I from the Critical Data Tables of the Corn Refiners Association, Inc. To convert RIₜₒₜ to DS, more than one table can be used and an interpolation between the 32 DE and 95 DE tables can specifically be employed. First an estimation of the solubilization is made by dividing the DS from the supernatant by the starting DS*1.1. The starting DS is the target dry substance starch slurry in the preparation and typically confirmed by Baume/DS tables or by dry substance determined on the original slurry by loss on drying (infrared balance). This estimated solubilization is used for the interpolation between the DS obtained via the 95DE and 32DE table. Solubilization is defined as the dry substance of the supernatant divided by the total dry substance times 100. This value is then corrected to compensate for the impact of remaining granular starch. This correction compensates for the water uptake by partial swelling and hydrolysis of starch granules remaining in the spin tube from the DS determination for supernatant.

SLS is generally run as a continuous process. SLS may be performed at any temperature above starch gelatinization wherein the glucoamylase is active. For example, SLS may be performed at a temperature between about 65° C. to about 70° C., about 75° C., about 80° C. Saccharification is normally conducted in stirred tanks, which may take several hours to fill or empty. Enzymes typically are added either at a fixed ratio to dried solids as the tanks are filled or added as a single dose at the commencement of the filling stage. An SLS reaction to make a syrup typically is run over about 24-72 hours, for example, 24-48 hours. When a maximum or desired DE has been attained, the reaction is stopped by heating to 85° C. for 5 min., for example. Further incubation will result in a lower DE, eventually to about 90 DE, as accumulated glucose re-polymerizes to isomaltose and/or other reversion products via an enzymatic reversion reaction and/or with the approach of thermodynamic equilibrium. When using an AfGATR polypeptide or variants thereof, SLS optimally is conducted at a temperature range of about 65° C. to about 80° C. Saccharification may be conducted over a pH range of about pH 3.0 to about pH 7.5, e.g., pH 3.5-pH 7.0, pH 4.0-pH 6.7, or pH 5.0.

Commercially available alpha amylases contemplated for use in the methods of the invention include SPEZYME™ AA; SPEZYME™ Fred, SPEZYME® Xtra (DuPont Industrial Bioscience), TERMAMYL™ 120-L, LC, SC and SUPRA (Novozymes), and Fuelzyme® FI from Verenium. As understood by those in the art, the quantity of alpha amylase used in the methods of the present invention will depend on the enzymatic activity of the alpha amylase. In general, an amount of about 0.01 to 10.0 kg of the alpha amylase is added to a metric ton (MT) of the raw material, although in some embodiments the alpha amylase is added in an amount about 1 to 5.0 kg. In other embodiments, the alpha amylase is added in an amount of about 0.5 to 1.0 kg. In further embodiments, other quantities are utilized. For example, between about 0.1 to 1.0 kg; between about 0.1 to 0.6 kg; between about 0.2 to 0.6 kg and between about 0.4 to 0.6 kg of SPEZYME® Xtra or SPEZYME™ FRED can be added per metric ton of starch.

Some additional alpha amylases useful as secondary enzymes include those obtained from bacteria such as Bacillus (e.g. B. licheniformis, B. lentus, B. coagulans, B. amyloliquefaciens, B. stearothermophilus, B subtilis, and hybrids, mutants and variants thereof) (U.S. Pat. No. 5,763,385; U.S. Pat. No. 5,824,532; U.S. Pat. No. 5,958,739; U.S. Pat. No. 6,008,026 and U.S. Pat. No. 6,361,809). Some of these amylases are commercially available e.g., TERMAMYL®, LIQUEZYME® SC and SUPRA®, Spirizyme Plus are available from Novozyme, Fuelzyme® LF from Verenium LLC and SPEZYME® Fred, SPEZYME® Xtra, SPEZYME® Alpha and SPEZYME® RSL are available from DuPont Industrial Bioscience.

AfGATR or a variant thereof may be added to the slurry in the form of a composition. AfGATR or a variant thereof can be added to a slurry of a granular starch substrate in an amount of about 0.6-10 ppm ds, e.g., 2 ppm ds. The AfGATR or variant thereof can be added as a whole broth, clarified, partially purified, or purified enzyme. The specific activity of the purified AfGA1TR or variant thereof may be about 187.7 U/mg, for example, measured with the ABTS assay. The specific activity of the purified AfGA2TR or variant thereof may be about 213.7 U/mg, for example, measured with the ABTS assay. The AfGATR or variant thereof also can be added as a whole broth product.

AfGATR or a variant thereof may be added to the slurry as an isolated enzyme solution. For example, AfGATR or a variant thereof can be added in the form of a cultured cell material produced by host cells expressing the AfGATR or variant thereof. The host cell producing and secreting the AfGA1 or a variant may also express an additional enzyme, such as a glucoamylase. For example, U.S. Pat. No. 5,422,267 discloses the use of a glucoamylase in yeast for production of alcoholic beverages. For example, a host cell, e.g., Trichoderma reesei may be engineered to co-express AfGA1 or a variant thereof and an α-amylase, including, but not limited to AkAA, AcAmy1, native Trichoderma reesei α-amylase, or variants thereof during saccharification. The host cell can be genetically modified so as not to express its endogenous glucoamylase and/or other enzymes, proteins or other materials. The host cell can be engineered to express a broad spectrum of various saccharolytic enzymes. For example, the recombinant yeast host cell can comprise nucleic acids encoding a glucoamylase, an alpha-glucosidase, an enzyme that utilizes pentose sugar, an α-amylase, a pullulanase, an isoamylase, and/or an isopullulanase. See, e.g., WO 2011/153516 A2.

Final carbohydrate composition can be determined by High Pressure Liquid Chromatographic (HPLC) method. The composition of the reaction products of oligosaccharides was measured by high pressure liquid chromatographic method (Beckman System Gold 32 Karat Fullerton, Calif., USA) equipped with a HPLC column (Rezex 8 u8% H, Monosaccharides), maintained at 50° C. fitted with a refractive index (RI) detector (ERC-7515A, RI Detector from The Anspec Company, Inc.). Dilute sulfuric acid (0.01 N) was used as the mobile phase at a flow rate of 0.6 ml per minute. Twenty microliter of 4.0% solution was injected onto the column. The column separates based on the molecular weight of the saccharides. For example a designation of DP1 is a monosaccharide, such as glucose; a designation of DP2 is a di-saccharide, such as maltose; a designation of DP3 is a tri-saccharide, such as maltotriose and the designation “DP4+” is an oligosaccharide having a degree of polymerization (DP) of 4 or greater.

4.3. Isomerization

The soluble starch hydrolysate produced by treatment with AfGATR or variants thereof can be converted into high fructose starch-based syrup (HFSS), such as high fructose corn syrup (HFCS). This conversion can be achieved using a glucose isomerase, particularly a glucose isomerase immobilized on a solid support. The pH is increased to about 6.0 to about 8.0, e.g., pH 7.5, and Ca²⁺ is removed by ion exchange. Suitable isomerases include Sweetzyme®, IT (Novozymes A/S); G-zyme® IMGI, and G-zyme® G993, Ketomax®, G-zyme® G993, G-zyme® G993 liquid, and GenSweet® IGI. Following isomerization, the mixture typically contains about 40-45% fructose, e.g., 42% fructose.

4.4. Fermentation

The soluble starch hydrolysate, particularly a glucose rich syrup, can be fermented by contacting the starch hydrolysate with a fermenting organism typically at a temperature around 32° C., such as from 30° C. to 35° C. EOF products include metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol and other biomaterials.

Ethanologenic microorganisms include yeast, such as Saccharomyces cerevisiae and bacteria, e.g., Zymomonas moblis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27(7): 1049-56. Commercial sources of yeast include ETHANOL RED® (LeSaffre); Thermosacc® (Lallemand); RED STAR® (Red Star); FERMIOL® (DSM Specialties); and SUPERSTART® (Alltech). Microorganisms that produce other metabolites, such as citric acid and lactic acid, by fermentation are also known in the art. See, e.g., Papagianni (2007) “Advances in citric acid fermentation by Aspergillus niger: biochemical aspects, membrane transport and modeling,” Biotechnol. Adv. 25(3): 244-63; John et al. (2009) “Direct lactic acid fermentation: focus on simultaneous saccharification and lactic acid production,” Biotechnol. Adv. 27(2): 145-52.

The saccharification and fermentation processes may be carried out as an SSF process. Fermentation may comprise subsequent purification and recovery of ethanol, for example. During the fermentation, the ethanol content of the broth or “beer” may reach about 8-18% v/v, e.g., 14-15% v/v. The broth may be distilled to produce enriched, e.g., 96% pure, solutions of ethanol. Further, CO₂ generated by fermentation may be collected with a CO₂ scrubber, compressed, and marketed for other uses, e.g., carbonating beverage or dry ice production. Solid waste from the fermentation process may be used as protein-rich products, e.g., livestock feed.

As mentioned above, an SSF process can be conducted with fungal cells, such as Trichoderma reesei, that express and secrete AfGATR or its variants continuously throughout SSF. The fungal cells expressing AfGATR or its variants also can be the fermenting microorganism, e.g., an ethanologenic microorganism. Ethanol production thus can be carried out using a fungal cell that expresses sufficient AfGATR or its variants so that less or no enzyme has to be added exogenously. The fungal host cell can be from an appropriately engineered fungal strain. Fungal host cells that express and secrete other enzymes, in addition to AfGATR or its variants, also can be used. Such cells may express α-amylase and/or a pullulanase, phytase, alpha-glucosidase, isoamylase, beta-amylase cellulase, xylanase, other hemicellulases, protease, beta-glucosidase, pectinase, esterase, redox enzymes, transferase, a glucoamylase other than AfGATR or other enzyme.

A variation on this process is a “fed-batch fermentation” system, where the substrate is added in increments as the fermentation progresses. Fed-batch systems are useful when catabolite repression may inhibit the metabolism of the cells and where it is desirable to have limited amounts of substrate in the medium. The actual substrate concentration in fed-batch systems is estimated by the changes of measurable factors such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density where cells are primarily in log phase growth. Continuous fermentation permits modulation of cell growth and/or product concentration. For example, a limiting nutrient such as the carbon source or nitrogen source is maintained at a fixed rate and all other parameters are allowed to moderate. Because growth is maintained at a steady state, cell loss due to medium being drawn off should be balanced against the cell growth rate in the fermentation. Methods of optimizing continuous fermentation processes and maximizing the rate of product formation are well known in the art of industrial microbiology.

4.6. Compositions Comprising AfGATR or Variants Thereof

AfGA1 or variants thereof may be combined with an α-amylase (EC 3.2.1.1). In some embodiments, the α-amylase is an acid stable alpha amylase which when added in an effective amount has activity in the pH range of 3.0 to 7.0 and preferably from 3.5 to 6.5. Alpha amylases useful according to the invention may be a fungal α-amylase or a bacterial α-amylase. Further the α-amylase may be a wild-type α-amylase or a variant thereof.

Preferred examples of fungal alpha amylases include those obtained from filamentous fungal strains including but not limited to strains of Aspergillus (e.g., A. niger, A. kawachii, and A. oryzae); Trichoderma sp., Rhizopus sp., Mucor sp., and Penicillium sp. Lactobacilli sp. and Streptomuces sp. The acid stable α-amylase may be derived from a bacterial strain. Preferred bacterial strains include Bacillus sp., such as B. licheniformis, B. stearothermophilus, B. amyloliquefaciens, B. subtilis, B. lentus, and B. coagulans. Particularly, B. licheniformis, B. stearothermophilus and B. amyloliquefaciens. One of the bacterial alpha amylases used in the compositions and processes of the invention may include one of the α-amylases described in U.S. Pat. Nos. 5,093,257; 5,763,385; 5,824,532; 5,958,739; 6,008,026; 6,093,563; 6,187,576; 6,361,809; 6,867,031; U.S. Publication No. 2006/0014265; International PCT Nos. WO 96/23874, WO 96/39528; WO 97/141213, WO 99/19467; and WO 05/001064.

Other suitable enzymes that can be used with AfGA1 or its variants include a glucoamylase that is not AfGA1, phytase, protease, pullulanase, β-amylase, isoamylase, α-amylase, alpha-glucosidase, cellulase, xylanase, other hemicellulases, beta-glucosidase, transferase, pectinase, lipase, cutinase, esterase, redox enzymes, or a combination thereof.

For example, a debranching enzyme, such as a pullulanase (EC 3.2.1.41), e.g., Promozyme®, may be added in effective amounts well known to the person skilled in the art. Pullulanase typically is added at 100 U/kg ds. Pullulanases are generally secreted by a Bacillus species. For example, Bacillus deramificans (U.S. Pat. No. 5,817,498; 1998), Bacillus acidopullulyticus (European Patent #0 063 909 and Bacillus naganoensis (U.S. Pat. No. 5,055,403). Enzymes having pullulanase activity used commercially are produced for examples, from Bacillus species (trade name OPTIMAX™ L-1000 from Danisco-Genencor and Promozyme™ from Novozymes).

Bacillus megaterium amylase/transferase (BMA): Bacillus megaterium amylase has the ability to convert the branched saccharides to a form that is easily hydrolyzed by glucoamylase. (Habeda R. E, Styrlund C. R and Teague, W. M.; 1988 Starch/Starke 40: 33-36). The enzyme exhibits maximum activity at pH 5.5 and temperature at 75° C. (David, M. H, Gunther H and Vilvoorde, H. R; 1987, Starch/Starke 39: 436-440). The enzyme has been cloned, expressed in a genetically engineered Bacillus subtilis and produced on a commercial scale (Brumm, P. J, Habeda R. E, and Teague W. M., 1991 Starch/Starke 43: 315-329). The enzyme is sold under a trade name MEGADEX™ for enhancing the glucose yield during the saccharification of enzyme liquefied starch by Aspergillus niger glucoamylase.

An isoamylase (EC 3.2.1.68), may also be added in effective amounts well known to the person skilled in the art. A pullulanase (EC 3.2.1.41), e.g., Promozyme®, is also suitable. Pullulanase typically is added at 100 U/kg ds. Further suitable enzymes include proteases, such as fungal and bacterial proteases. Fungal proteases include those obtained from Aspergillus, such as A. niger, A. awamori, A. oryzae; Mucor (e.g., M. miehei); Rhizopus; and Trichoderma.

β-Amylases (EC 3.2.1.2) are exo-acting maltogenic amylases, which catalyze the hydrolysis of 1,4-α-glucosidic linkages into amylopectin and related glucose polymers, thereby releasing maltose. β-Amylases have been isolated from various plants and microorganisms. See Fogarty et al. (1979) in PROGRESS IN INDUSTRIAL MICROBIOLOGY, Vol. 15, pp. 112-115. These β-Amylases have optimum temperatures in the range from 40° C. to 65° C. and optimum pH in the range from about 4.5 to about 7.0. Contemplated β-amylases include, but are not limited to, β-amylases from barley Spezyme® BBA 1500, Spezyme® DBA, Optimalt™ ME, Optimalt™ BBA (Danisco US Inc.); and Novozym™ WBA (Novozymes A/S).

AfGATR or variants thereof may be combined with an α-amylase (EC 3.2.1.1). In some embodiments, the α-amylase is an acid stable alpha amylase which when added in an effective amount has activity in the pH range of 3.0 to 7.0 and preferably from 3.5 to 6.5. Alpha amylases may be a fungal α-amylase or a bacterial α-amylase. Further, the α-amylase may be a wild-type α-amylase or a variant thereof.

Preferred examples of fungal alpha amylases include those obtained from filamentous fungal strains including but not limited to strains of Aspergillus (e.g., A. niger, A. kawachii, and A. oryzae); Trichoderma sp., Rhizopus sp., Mucor sp., and Penicillium sp. Lactobacilli sp. and Streptomuces sp. The acid stable α-amylase may be derived from a bacterial strain. Preferred bacterial strains include Bacillus sp., such as B. licheniformis, B. stearothermophilus, B. amyloliquefaciens, B. subtilis, B. lentus, and B. coagulans. Particularly, B. licheniformis, B. stearothermophilus, and B. amyloliquefaciens. One of the bacterial alpha amylases used in the compositions and processes of the invention may include one of the α-amylases described in U.S. Pat. Nos. 5,093,257; 5,763,385; 5,824,532; 5,958,739; 6,008,026; 6,093,563; 6,187,576; 6,361,809; 6,867,031; US Patent Publication No. 2006/0014265; International PCT Nos. WO 96/23874, WO 96/39528; WO 97/141213, WO 99/19467; and WO 05/001064.

Exemplary α-amylases include is AkAA or AcAmy1 and variants thereof that possess superior specific activity and thermal stability. Suitable variants of AkAA include those with α-amylase activity and at least 80%, 90%, 95%, 98%, or at least 99% sequence identity to wild-type AkAA. Suitable variants of AcAmy1 include those with α-amylase activity and at least 80%, at least 90%, or at least 95% sequence identity to wild-type AcAmy1. AfGATR and its variants advantageously increase the yield of glucose produced in a saccharification process catalyzed by AnGA or TrGA.

Commercially available alpha amylases contemplated for use in the compositions and method include: SPEZYME™ AA; SPEZYME™ FRED; SPEZYME™ XTRA; GZYME™ 997; and CLARASE™ L (Genencor International Inc.); TERMAMYL™ 120-L, LC and SC and SUPRA (Novozymes Biotech); LIQUOZYME™ X and SAN™ SUPER (Novozymes A/S) and Fuelzyme™ LF (Diversa). In some embodiments, the alpha amylase will include an alpha amylase derived from Bacillus stearothermophilus such as SPEZYME™ AA, SPEZYME™ FRED or SPEZYME™ XTRA. In some embodiments, the enzyme compositions will include BP-WT, SPEZYME™ XTRA and optionally SPEZYME™ FRED. In other embodiments, the compositions will include BP-17, SPEZYME™ XTRA and optionally SPEZYME™ FRED.

Other suitable enzymes that can be used with AfGATR or its variants include a glucoamylase that is not AfGATR, phytase, protease, pullulanase, β-amylase, isoamylase, α-amylase, alpha-glucosidase, cellulase, xylanase, other hemicellulases, beta-glucosidase, transferase, pectinase, lipase, cutinase, esterase, redox enzymes, or a combination thereof.

For example, a debranching enzyme, such as a pullulanase (EC 3.2.1.41), e.g., Promozyme®, may be added in effective amounts well known to the person skilled in the art. Pullulanase typically is added at 100 U/kg ds. Pullulanases are generally secreted by a Bacillus species. Exemplary pullulanases are described for Bacillus deramificans (U.S. Pat. No. 5,817,498; 1998), Bacillus acidopullulyticus (European Patent #0 063 909 and Bacillus naganoensis (U.S. Pat. No. 5,055,403). Enzymes having pullulanase activity used commercially are produced for examples, from Bacillus species (trade name OPTIMAX™ L-1000 from Danisco-Genencor and Promozyme™ from Novozymes).

Bacillus megaterium amylase/transferase (BMA): Bacillus megaterium amylase has the ability to convert the branched saccharides to a form that is easily hydrolyzed by glucoamylase (Habeda R. E, Styrlund C. R and Teague, W. M.; 1988 Starch/Starke, 40: 33-36). The enzyme exhibits maximum activity at pH 5.5 and temperature at 75 C. (David, M. H, Gunther H and Vilvoorde, H. R; 1987, Starch/Starke, 39: 436-440). The enzyme has been cloned, expressed in a genetically engineered Bacillus subtilis and produced on a commercial scale (Brumm, P. J, Habeda R. E, and Teague W. M, 1991 Starch/Starke, 43: 315-329). The enzyme is sold under a trade name MEGADEX™ for enhancing the glucose yield during the saccharification of enzyme-liquefied starch by Aspergillus niger glucoamylase.

An isoamylase (EC 3.2.1.68), may also be added in effective amounts well known to the person skilled in the art. A pullulanase (EC 3.2.1.41), e.g., Promozyme®, is also suitable. Pullulanase typically is added at 100 U/kg ds. Further suitable enzymes include proteases, such as fungal and bacterial proteases. Fungal proteases include those obtained from Aspergillus, such as A. niger, A. awamori, A. oryzae; Mucor (e.g., M. miehei); Rhizopus; and Trichoderma.

β-Amylases (EC 3.2.1.2) are exo-acting maltogenic amylases, which catalyze the hydrolysis of 1,4-α-glucosidic linkages into amylopectin and related glucose polymers, thereby releasing maltose. β-Amylases have been isolated from various plants and microorganisms. See Fogarty et al. (1979) in PROGRESS IN INDUSTRIAL MICROBIOLOGY, Vol. 15, pp. 112-115. These β-Amylases have optimum temperatures in the range from 40° C. to 65° C. and optimum pH in the range from about 4.5 to about 7.0. Contemplated β-amylases include, but are not limited to, β-amylases from barley Spezyme® BBA 1500, Spezyme® DBA, Optimalt™ ME, Optimalt™ BBA (Danisco US Inc.); and Novozym™ WBA (Novozymes A/S).

5. Compositions and Methods for Baking and Food Preparation

The present invention also relates to a “food composition,” including but not limited to a food product, animal feed and/or food/feed additives, comprising a glucose composition produced by the disclosed SLS methods, and methods for preparing such a food composition comprising mixing the glucose composition with one or more food ingredients, or uses thereof. An AfGATR or variant thereof can also be mixed into the food composition.

Furthermore, the present invention relates to the use the glucose composition produced by the disclosed SLS methods in the preparation of a food composition, wherein the food composition is baked subsequent to the addition of the polypeptide of the invention. As used herein the term “baking composition” means any composition and/or additive prepared in the process of providing a baked food product, including but not limited to bakers flour, a dough, a baking additive and/or a baked product. The food composition or additive may be liquid or solid.

As used herein, the term “flour” means milled or ground cereal grain. The term “flour” also may mean Sago or tuber products that have been ground or mashed. In some embodiments, flour may also contain components in addition to the milled or mashed cereal or plant matter. An example of an additional component, although not intended to be limiting, is a leavening agent. Cereal grains include wheat, oat, rye, and barley. Tuber products include tapioca flour, cassava flour, and custard powder. The term “flour” also includes ground corn flour, maize-meal, rice flour, whole-meal flour, self-rising flour, tapioca flour, cassava flour, ground rice, enriched flower, and custard powder.

For the commercial and home use of flour for baking and food production, it is important to maintain an appropriate level of glucoamylase activity in the flour. A level of activity that is too high may result in a product that is sticky and/or doughy and therefore unmarketable. Flour with insufficient glucoamylase activity may not contain enough sugar for proper yeast function, resulting in dry, crumbly bread, or baked products. Accordingly, an AfGATR or variant thereof, by itself or in combination with an α-amylase(s), may be added to the flour to augment the level of endogenous glucoamylase activity in flour.

The glucose composition produced by the disclosed SLS methods can be added alone or in a combination with other amylases to prevent or retard staling, i.e., crumb firming of baked products. The amount of anti-staling amylase will typically be in the range of 0.01-10 mg of enzyme protein per kg of flour, e.g., 0.5 mg/kg ds. Additional anti-staling amylases that can be used in combination with an AfGATR or variant thereof include an endo-amylase, e.g., a bacterial endo-amylase from Bacillus. The additional amylase can be another maltogenic α-amylase (EC 3.2.1.133), e.g., from Bacillus. Novamyl® is an exemplary maltogenic α-amylase from B. stearothermophilus strain NCIB 11837 and is described in Christophersen et al. (1997) Starch 50: 39-45. Other examples of anti-staling endo-amylases include bacterial α-amylases derived from Bacillus, such as B. licheniformis or B. amyloliquefaciens. The anti-staling amylase may be an exo-amylase, such as β-amylase, e.g., from plant sources, such as soy bean, or from microbial sources, such as Bacillus.

The baking composition comprising a glucose composition produced by the disclosed SLS methods can methods can further comprise a phospholipase or enzyme with phospholipase activity. An enzyme with phospholipase activity has an activity that can be measured in Lipase Units (LU). The phospholipase may have A₁ or A₂ activity to remove fatty acid from the phospholipids, forming a lysophospholipid. It may or may not have lipase activity, i.e., activity on triglyceride substrates. The phospholipase typically has a temperature optimum in the range of 30 to 90° C., e.g., 30 to 70° C. The added phospholipases can be of animal origin, for example, from pancreas, e.g., bovine or porcine pancreas, snake venom or bee venom. Alternatively, the phospholipase may be of microbial origin, e.g., from filamentous fungi, yeast or bacteria, for example.

The phospholipase is added in an amount that improves the softness of the bread during the initial period after baking, particularly the first 24 hours. The amount of phospholipase will typically be in the range of 0.01-10 mg of enzyme protein per kg of flour, e.g., 0.1-5 mg/kg. That is, phospholipase activity generally will be in the range of 20-1000 LU/kg of flour, where a Lipase Unit is defined as the amount of enzyme required to release 1 μmol butyric acid per minute at 30° C., pH 7.0, with gum arabic as emulsifier and tributyrin as substrate.

Compositions of dough generally comprise wheat meal or wheat flour and/or other types of meal, flour or starch such as corn flour, cornstarch, rye meal, rye flour, oat flour, oatmeal, soy flour, sorghum meal, sorghum flour, potato meal, potato flour or potato starch. The dough may be fresh, frozen or par-baked. The dough can be a leavened dough or a dough to be subjected to leavening. The dough may be leavened in various ways, such as by adding chemical leavening agents, e.g., sodium bicarbonate or by adding a leaven, i.e., fermenting dough. Dough also may be leavened by adding a suitable yeast culture, such as a culture of Saccharomyces cerevisiae (baker's yeast), e.g., a commercially available strain of S. cerevisiae.

The dough may also comprise other conventional dough ingredients, e.g., proteins, such as milk powder, gluten, and soy; eggs (e.g., whole eggs, egg yolks or egg whites); an oxidant, such as ascorbic acid, potassium bromate, potassium iodate, azodicarbonamide (ADA) or ammonium persulfate; an amino acid such as L-cysteine; a sugar; or a salt, such as sodium chloride, calcium acetate, sodium sulfate or calcium sulfate. The dough further may comprise fat, e.g., triglyceride, such as granulated fat or shortening. The dough further may comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin. In particular, the dough can be made without addition of emulsifiers.

The dough product may be any processed dough product, including fried, deep fried, roasted, baked, steamed and boiled doughs, such as steamed bread and rice cakes. In one embodiment, the food product is a bakery product. Typical bakery (baked) products include bread—such as loaves, rolls, buns, bagels, pizza bases etc. pastry, pretzels, tortillas, cakes, cookies, biscuits, crackers etc.

Optionally, an additional enzyme may be used together with the anti-staling amylase and the phospholipase. The additional enzyme may be a second amylase, such as an amyloglucosidase, a β-amylase, a cyclodextrin glucanotransferase, or the additional enzyme may be a peptidase, in particular an exopeptidase, a transglutaminase, a lipase, a cellulase, a xylanase, a protease, a protein disulfide isomerase, e.g., a protein disulfide isomerase as disclosed in WO 95/00636, for example, a glycosyltransferase, a branching enzyme (1,4-α-glucan branching enzyme), a 4-α-glucanotransferase (dextrin glycosyltransferase) or an oxidoreductase, e.g., a peroxidase, a laccase, a glucose oxidase, a pyranose oxidase, a lipooxygenase, an L-amino acid oxidase or a carbohydrate oxidase. The additional enzyme(s) may be of any origin, including mammalian and plant, and particularly of microbial (bacterial, yeast or fungal) origin and may be obtained by techniques conventionally used in the art.

The xylanase is typically of microbial origin, e.g., derived from a bacterium or fungus, such as a strain of Aspergillus. Xylanases include Pentopan® and Novozym 384®, for example, which are commercially available xylanase preparations produced from Trichoderma reesei. The amyloglucosidase may be an A. niger amyloglucosidase (such as AMG®). Other useful amylase products include Grindamyl® A 1000 or A 5000 (Grindsted Products, Denmark) and Amylase® H or Amylase® P (DSM). The glucose oxidase may be a fungal glucose oxidase, in particular an Aspergillus niger glucose oxidase (such as Gluzyme®). An exemplary protease is Neutrase®.

The process may be used for any kind of baked product prepared from dough, either of a soft or a crisp character, either of a white, light or dark type. Examples are bread, particularly white, whole-meal or rye bread, typically in the form of loaves or rolls, such as, but not limited to, French baguette-type bread, pita bread, tortillas, cakes, pancakes, biscuits, cookies, pie crusts, crisp bread, steamed bread, pizza and the like.

The glucose composition produced by the disclosed SLS methods may be used in a pre-mix, comprising flour together with an anti-staling amylase, a phospholipase, and/or a phospholipid. The pre-mix may contain other dough-improving and/or bread-improving additives, e.g., any of the additives, including enzymes, mentioned above. The AfGATR or variant thereof can additionally be a component of an enzyme preparation comprising an anti-staling amylase and a phospholipase, for use as a baking additive. The enzyme preparation is optionally in the form of a granulate or agglomerated powder. The preparation can have a narrow particle size distribution with more than 95% (by weight) of the particles in the range from 25 to 500 μm. Granulates and agglomerated powders may be prepared by conventional methods, e.g., by spraying the AfGATR or variant thereof onto a carrier in a fluid-bed granulator. The carrier may consist of particulate cores having a suitable particle size. The carrier may be soluble or insoluble, e.g., a salt (such as NaCl or sodium sulfate), a sugar (such as sucrose or lactose), a sugar alcohol (such as sorbitol), starch, rice, corn grits, or soy.

Enveloped particles, i.e., glucoamylase particles, can comprise an AfGATR or variants thereof. To prepare enveloped glucoamylase particles, the enzyme is contacted with a food grade lipid in sufficient quantity to suspend all of the glucoamylase particles. Food grade lipids, as used herein, may be any naturally organic compound that is insoluble in water but is soluble in non-polar organic solvents such as hydrocarbon or diethyl ether. Suitable food grade lipids include, but are not limited to, triglycerides either in the form of fats or oils that are either saturated or unsaturated. Examples of fatty acids and combinations thereof which make up the saturated triglycerides include, but are not limited to, butyric (derived from milk fat), palmitic (derived from animal and plant fat), and/or stearic (derived from animal and plant fat). Examples of fatty acids and combinations thereof which make up the unsaturated triglycerides include, but are not limited to, palmitoleic (derived from animal and plant fat), oleic (derived from animal and plant fat), linoleic (derived from plant oils), and/or linolenic (derived from linseed oil). Other suitable food grade lipids include, but are not limited to, monoglycerides and diglycerides derived from the triglycerides discussed above, phospholipids and glycolipids.

The food grade lipid, particularly in the liquid form, is contacted with a powdered form of the glucoamylase particles in such a fashion that the lipid material covers at least a portion of the surface of at least a majority, e.g., 100% of the glucoamylase particles. Thus, each glucoamylase particle is individually enveloped in a lipid. For example, all or substantially all of the glucoamylase particles are provided with a thin, continuous, enveloping film of lipid. This can be accomplished by first pouring a quantity of lipid into a container, and then slurrying the glucoamylase particles so that the lipid thoroughly wets the surface of each glucoamylase particle. After a short period of stirring, the enveloped glucoamylase particles, carrying a substantial amount of the lipids on their surfaces, are recovered. The thickness of the coating so applied to the particles of glucoamylase can be controlled by selection of the type of lipid used and by repeating the operation in order to build up a thicker film, when desired.

The storing, handling and incorporation of the loaded delivery vehicle can be accomplished by means of a packaged mix. The packaged mix can comprise the enveloped α-amylase. However, the packaged mix may further contain additional ingredients as required by the manufacturer or baker. After the enveloped glucoamylase has been incorporated into the dough, the baker continues through the normal production process for that product.

The advantages of enveloping the glucoamylase particles are two-fold. First, the food grade lipid protects the enzyme from thermal denaturation during the baking process for those enzymes that are heat labile. Consequently, while the glucoamylase is stabilized and protected during the proving and baking stages, it is released from the protective coating in the final baked good product, where it hydrolyzes the glucosidic linkages in polyglucans. The loaded delivery vehicle also provides a sustained release of the active enzyme into the baked good. That is, following the baking process, active glucoamylase is continually released from the protective coating at a rate that counteracts, and therefore reduces the rate of, staling mechanisms.

In general, the amount of lipid applied to the glucoamylase particles can vary from a few percent of the total weight of the glucoamylase to many times that weight, depending upon the nature of the lipid, the manner in which it is applied to the glucoamylase particles, the composition of the dough mixture to be treated, and the severity of the dough-mixing operation involved.

The loaded delivery vehicle, i.e., the lipid-enveloped enzyme, is added to the ingredients used to prepare a baked good in an effective amount to extend the shelf-life of the baked good. The baker computes the amount of enveloped α-amylase, prepared as discussed above, that will be required to achieve the desired anti-staling effect. The amount of the enveloped glucoamylase required is calculated based on the concentration of enzyme enveloped and on the proportion of glucoamylase to flour specified. A wide range of concentrations has been found to be effective, although, as has been discussed, observable improvements in anti-staling do not correspond linearly with the glucoamylase concentration, but above certain minimal levels, large increases in glucoamylase concentration produce little additional improvement. The glucoamylase concentration actually used in a particular bakery production could be much higher than the minimum necessary to provide the baker with some insurance against inadvertent under-measurement errors by the baker. The lower limit of enzyme concentration is determined by the minimum anti-staling effect the baker wishes to achieve.

A method of preparing a baked good may comprise: a) preparing lipid-coated glucoamylase particles, where substantially all of the glucoamylase particles are coated; b) mixing a dough containing flour; c) adding the lipid-coated glucoamylase to the dough before the mixing is complete and terminating the mixing before the lipid coating is removed from the α-amylase; d) proofing the dough; and e) baking the dough to provide the baked good, where the glucoamylase is inactive during the mixing, proofing and baking stages and is active in the baked good.

The enveloped glucoamylase can be added to the dough during the mix cycle, e.g., near the end of the mix cycle. The enveloped glucoamylase is added at a point in the mixing stage that allows sufficient distribution of the enveloped glucoamylase throughout the dough; however, the mixing stage is terminated before the protective coating becomes stripped from the glucoamylase particle(s). Depending on the type and volume of dough, and mixer action and speed, anywhere from one to six minutes or more might be required to mix the enveloped glucoamylase into the dough, but two to four minutes is average. Thus, several variables may determine the precise procedure. First, the quantity of enveloped glucoamylase should have a total volume sufficient to allow the enveloped glucoamylase to be spread throughout the dough mix. If the preparation of enveloped glucoamylase is highly concentrated, additional oil may need to be added to the pre-mix before the enveloped glucoamylase is added to the dough. Recipes and production processes may require specific modifications; however, good results generally can be achieved when 25% of the oil specified in a bread dough formula is held out of the dough and is used as a carrier for a concentrated enveloped glucoamylase when added near the end of the mix cycle. In bread or other baked goods, particularly those having a low fat content, e.g., French-style breads, an enveloped glucoamylase mixture of approximately 1% of the dry flour weight is sufficient to admix the enveloped glucoamylase properly with the dough. The range of suitable percentages is wide and depends on the formula, finished product, and production methodology requirements of the individual baker. Second, the enveloped glucoamylase suspension should be added to the mix with sufficient time for complete mixture into the dough, but not for such a time that excessive mechanical action strips the protective lipid coating from the enveloped glucoamylase particles.

In a further aspect of the invention, the food composition is an oil, meat, lard, composition comprising the glucose composition produced by the disclosed SLS methods. In this context the term “[oil/meat/lard] composition” means any composition, based on, made from and/or containing oil, meat or lard, respectively. Another aspect the invention relates to a method of preparing an oil or meat or lard composition and/or additive comprising an AfGATR or a variant thereof, comprising mixing the polypeptide of the invention with a oil/meat/lard composition and/or additive ingredients.

In a further aspect of the invention, the food composition is an animal feed composition, animal feed additive and/or pet food comprising the glucose composition produced by the disclosed SLS methods. The present invention further relates to a method for preparing such an animal feed composition, animal feed additive composition and/or pet food comprising mixing the glucose composition produced by the disclosed SLS methods with one or more animal feed ingredients and/or animal feed additive ingredients and/or pet food ingredients. Furthermore, the present invention relates to the use of the glucose composition produced by the disclosed SLS methods in the preparation of an animal feed composition and/or animal feed additive composition and/or pet food.

The term “animal” includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feedstuff” and “fodder” are used interchangeably and may comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

6. Brewing Compositions

The glucose composition produced by the disclosed SLS methods may be a component of a brewing composition used in a process of providing a fermented beverage, such as brewing. It is believed that non-fermentable carbohydrates form the majority of the dissolved solids in the final beer. This residue remains because of the inability of malt amylases to hydrolyze the alpha-1,6-linkages of the starch. The non-fermentable carbohydrates contribute about 50 calories per 12 ounces (about 340 grams) of beer. The glucose composition produced by the disclosed SLS methods, usually in combination with a glucoamylase like AfGATR and optionally a pullulanase and/or isoamylase, assist in converting the starch into dextrins and fermentable sugars, lowering the residual non-fermentable carbohydrates in the final beer.

The principal raw materials used in making these beverages are water, hops and malt. In addition, but also exclusively, adjuncts such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch.

For a number of reasons, the malt, which is produced principally from selected varieties of barley, has an important effect on the overall character and quality of the beer. First, the malt is the primary flavoring agent in beer. Second, the malt provides the major portion of the fermentable sugar. Third, the malt provides the proteins, which will contribute to the body and foam character of the beer. Fourth, the malt provides the necessary enzymatic activity during mashing. Hops also contribute significantly to beer quality, including flavoring. In particular, hops (or hops constituents) add desirable bittering substances to the beer. In addition, the hops can act as protein precipitants, establish preservative agents and aid in foam formation and stabilization.

Cereals (grains), such as barley, oats, wheat, but also corn and rice are often used for brewing, both in industry and for home brewing, but also other plant components, such as hops are often added. The components used in brewing may be unmalted or may be malted, i.e., partially germinated, resulting in an increase in the levels of enzymes, including α-amylase. For successful brewing, adequate levels of α-amylase enzyme activity are necessary to ensure the appropriate levels of sugars for fermentation. An AfGATR or variant thereof may also be added to the components used for brewing.

As used herein, the term “stock” means grains and plant components that are crushed or broken. For example, barley used in beer production is a grain that has been coarsely ground or crushed to yield a consistency appropriate for producing a mash for fermentation. As used herein, the term “stock” includes any of the aforementioned types of plants and grains in crushed or coarsely ground forms. The methods described herein may be used to determine α-amylase activity levels in both flours and stock.

Processes for making beer are well known in the art. See, e.g., Wolfgang Kunze (2004) “Technology Brewing and Malting,” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer. Typically, milled or crushed malt, malt and adjunct, or adjunct is mixed with water and held for a period of time under controlled temperatures to permit the enzymes present in the malt and/or adjunct to convert the starch present in the malt into fermentable sugars. The mash is then transferred to a mash filter where the liquid is separated from the grain residue. This sweet liquid is called “wort,” and the left over grain residue is called “spent grain.” The mash is typically subjected to an extraction, which involves adding water to the mash in order to recover the residual soluble extract from the spent grain. The wort is then boiled vigorously to sterilize the wort and help develop the color, flavor and odor. Hops are added at some point during the boiling. The wort is cooled and transferred to a fermenter.

The wort is then contacted in a fermenter with yeast. The fermenter may be chilled to stop fermentation. The yeast which may flocculate is removed. Finally, the beer is cooled and stored for a period of time, during which the beer clarifies and its flavor develops, and any material that might impair the appearance, flavor and shelf life of the beer settles out. The beer usually contains from about 2% to about 10% v/v alcohol, although beer with a higher alcohol content, e.g., 18% v/v, may be obtained. Prior to packaging, the beer is carbonated and, optionally, filtered and pasteurized.

The brewing composition comprising the glucose composition produced by the disclosed SLS methods and an alpha-amylase, often, but not necessarily in combination with one or more exogenous enzymes, such as glucoamylase(s) (e.g. AfGATR), and optionally pullulanase(s) and/or isoamylase(s) and any combination thereof, may be added to the mash of step (a) above, such as during the preparation of the mash. Alternatively, or in addition, the brewing composition may be added to the mash of step (b) above, i.e., during the filtration of the mash. Alternatively, or in addition, the brewing composition may be added to the wort of step (c) above, such as during the fermenting of the wort.

Particular embodiments pertains to any of the above uses, methods or fermented beverages, wherein said fermented beverage is a beer, such as full malted beer, beer brewed under the “Reinheitsgebot,” ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavored malt beverages, e.g., citrus flavored, such as lemon-, orange-, lime-, or berry-flavored malt beverages, liquor flavored malt beverages, e.g., vodka-, rum-, or tequila-flavored malt liquor, or coffee flavored malt beverages, such as caffeine-flavored malt liquor, and the like.

7. Reduction of Iodine-Positive Starch

AfGATR and variants thereof may reduce the iodine-positive starch (IPS), when used in SLS. One source of IPS is from amylose that escapes hydrolysis and/or from retrograded starch polymer. Starch retrogradation occurs spontaneously in a starch paste, or gel on ageing, because of the tendency of starch molecules to bind to one another followed by an increase in crystallinity. Solutions of low concentration become increasingly cloudy due to the progressive association of starch molecules into larger articles. Spontaneous precipitation takes place and the precipitated starch appears to be reverting to its original condition of cold-water insolubility. Pastes of higher concentration on cooling set to a gel, which on ageing becomes steadily firmer due to the increasing association of the starch molecules. This arises because of the strong tendency for hydrogen bond formation between hydroxy groups on adjacent starch molecules. See J. A. Radley, ed., Starch and its Derivatives, 194-201 (Chapman and Hall, London (1968)).

The presence of IPS in saccharide liquor negatively affects final product quality and represents a major issue with downstream processing. IPS plugs or slows filtration system, and fouls the carbon columns used for purification. When IPS reaches sufficiently high levels, it may leak through the carbon columns and decrease production efficiency. Additionally, it may results in hazy final product upon storage, which is unacceptable for final product quality. The amount of IPS can be reduced by isolating the saccharification tank and blending the contents back. IPS nevertheless will accumulate in carbon columns and filter systems, among other things. The use of AfGATR or variants thereof thus is expected to improve overall process performance by reducing the amount of IPS.

### EXAMPLES

### Example 1

### Cloning of AfGA1

Genomic DNA of Aspergillus fumigatus Af293 was purchased from Fungal Genetics Stock Center, Kansas City, Mo. (FGSC A1100). The genome of Aspergillus fumigatus is sequenced. The nucleic acid sequence for the AfGA1 gene (within the disclosed genome in NCBI Reference Sequence NC_007195), and the amino acid sequence of the predicted glucan 1,4-alpha-glucosidase (NCBI Accession No. XP_749206) encoded by the AfGA1 gene were obtained in the NCBI Databases. AfGA1 is homologous to other fungal glucoamylases as determined from a BLAST search. See FIG. 1. The nucleotide sequence of the AfGA1 gene, which comprises three introns, is set forth in SEQ ID NO: 8.

The AfGA1 gene was amplified from genomic DNA of Aspergillus fumigatus using the following primers: Primer 1: AfGA1-Fw 5′-GCG GCGGCCGC ACC atgcctcgcctttcctacgc-3′ (SEQ ID NO: 9), and Primer 2: AfGA1-Rv 5′-cc ggcgcgccc TTA tcactgccaagtatcattctcg-3′ (SEQ ID NO: 10). The forward primer contains a NotI restriction site, and the reverse primer contains an AscI restriction site. After digestion with Not I and Asc I, the PCR product was cloned into pTrex3gM expression vector (described in U.S. Published Application 2011/0136197 A1) digested with the same restriction enzymes, and the resulting plasmid was labeled pJG222. A plasmid map of pJG222 is provided in FIG. 2. The sequence of the AfGA1 gene was confirmed by DNA sequencing.

### Example 2

### Expression and Purification of AfGA1TR

The plasmid pJG222 (Trex3gM-AfGA1) was transformed into a quad-deleted Trichoderma reesei strain (described in WO 05/001036) using biolistic method (Te'o et al., J. Microbiol. Methods 51:393-99, 2002). Transformed colonies (about 50) appeared in about 1 week. After growth on acetamide plates for 5 days, the colonies were inoculated in 250 ml shake flasks with 30 ml Glucose/Sephorose defined medium for protein expression. The protein, AfGA1TR, was secreted into the extracellular medium, and the filtered culture medium was used to perform SDS-PAGE and a glucoamylase activity on DP7 assay to confirm the enzyme expression.

The stable strain was subsequently grown in a 7 L fermenter in a defined medium. Fermentation broth was harvested by centrifugation. Following centrifugation, filtration and concentration, 450 ml of the concentrated sample was obtained. The concentration of total protein in the sample was determined to be 83.70 g/L by using BCA method (protein quantification kit, Shanghai Generay Biotech CO., Ltd). SDS-PAGE analysis suggested that 80% of the total protein was the target protein. Thus, the concentration of target protein in the concentrated sample was estimated to be 66.96 g/L.

AfGA1TR was purified by affinity chromatography using an AKTA Explorer 100 FPLC system (GE Healthcare). β-Cyclodextrin (Sigma-Aldrich, 856088) was coupled to epoxy-activated Sepharose beads (GE Healthcare, 17-0480-01) and employed for purification. The pH of 40 ml concentrated fermentation broth from the 7 L fermenter was adjusted to 4.3 and the solution was loaded onto 30 ml β-CD-Sepharose column pre-equilibrated with 25 mM, pH 4.3 sodium acetate (buffer A). The column was washed with 2 column volume of buffer A. The target protein was eluted with three column volume of buffer B which containing buffer A and 10 mM α-cyclodextrin (Sigma-Aldrich, C4642). Fractions were analyzed by SDS-PAGE gel and assayed for glucoamylase activity. The fractions containing target protein were pooled and run through a Hiprep 26×10 desalting column to remove β-cylcodextrin. The resulting sample was more than 95% pure, the solution was concentrated using 10K Amicon Ultra-15 devices and stored in 40% glycerol at −80° C.

### Example 3

### Determination of AfGA1TR Substrate Specificity

Glucoamylase activity was assayed based on the release of glucose by glucoamylases, AfGA1TR, AnGA or wild-type AfGA, from different substrates, including maltose, isomaltose, maltoheptaose (DP7), maltodextrin (DE4-DE10), potato amylopectin, and soluble starch. The rate of glucose release was measured using a coupled glucose oxidase/peroxidase (GOX/HRP) method (Anal. Biochem. 105 (1980), 389-397). Glucose was quantified as the rate of oxidation of 2,2′-Azino-bis 3-ethylbenzothiazoline-6-sulfonic acid (ABTS) by peroxide which was generated from coupled GOX/HRP enzymes reacted with glucose.

Substrate solutions were prepared by mixing 9 mL of each substrate (1% in water, w/w) and 1 mL of 0.5 M pH 5.0 sodium acetate buffer in a 15-mL conical tube. Coupled enzyme (GOX/HRP) solution with ABTS was prepared by dissolving GOX/HRP in 50 mM sodium acetate buffer (pH 5.0), with the final concentrations of 2.74 mg/mL ABTS, 0.1 U/mL HRP, and 1 U/mL GOX.

Serial dilutions of glucoamylase samples, the benchmark AnGA (Genencor product, Optidex L-400), wild-type AfGA, and glucose standard were also prepared in 50 mM sodium acetate buffer (pH 5.0). Each glucoamylase sample (10 μl) was transferred into a new microtiter plate (Corning 3641) containing 90 μl of substrate solution preincubated at 50° C. for 5 min at 600 rpm. The reactions were carried out at 50° C. for 10 min with shaking (600 rpm) in a thermomixer (Eppendorf), 10 μl of reaction mixtures as well as 10 μl of serial dilutions of glucose standard were quickly transferred to new microtiter plates (Corning 9017), respectively, followed by the addition of 100 μl of ABTS/GOX/HRP solution. The microtiter plates containing the reaction mixture were immediately measured at 405 nm at 11 seconds intervals for 5 min on SoftMax Pro plate reader (Molecular Device). The output was the reaction rate, Vo, for each enzyme concentration. Linear regression was used to determine the slope of the plot Vo vs. enzyme dose. The specific activity of glucoamylase activity was calculated based on the glucose standard curve using Equation 1:

Specific Activity(Unit/mg)=Slope(enzyme)/slope(std)×100 (1),
where 1 Unit=1 μmol glucose/min.

Representative specific activities of AfGA1TR and the benchmark glucoamylases AnGA and wild-type/AfGA are shown in Table 2.

TABLE 2
Specific activity of purified glucoamylases on various substrates.

| | Specific activity (U/mg) | | |
|---|---|---|---|
| Substrate | AnGA | Wild-type AfGA | AfGA1TR |
| Maltose (DP2) | 29.2 | 29.2 | 42.7 |
| Isomaltose | 0 | 0.6 | 0.9 |
| Maltoheptaose (DP7) | 159.9 | 180.3 | 254.8 |
| Maltodextrin (DE4-DE10) | 128.8 | 127.8 | 211.5 |
| Amylopectin from | 142.5 | 146.5 | 197.8 |
| potato | | | |
| Soluble starch | 137.5 | 128.0 | 213.4 |
| Pullulan | 29.2 | 25.6 | 31.1 |

### Example 4

### Effect of pH on AfGA1TR Glucoamylase Activity

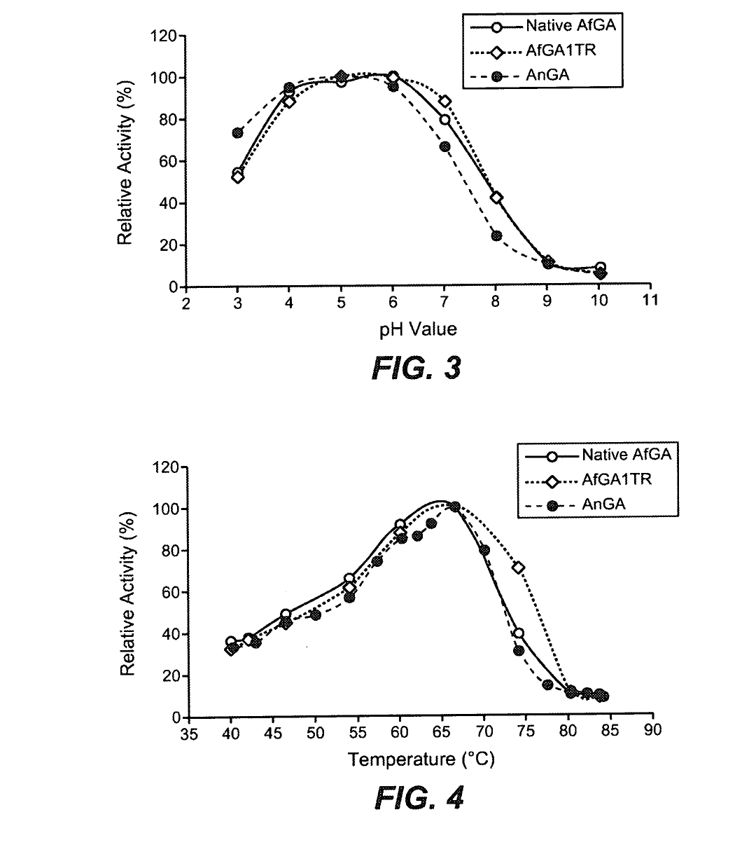

The effect of pH (from 3.0 to 10.0) on AfGA1TR activity was monitored using the ABTS assay protocol as described in Example 3. Buffer working solutions consisted of the combination of glycine/sodium acetate/HEPES (250 mM), with pH varying from 3.0 to 10.0. Substrate solutions were prepared by mixing soluble starch (1% in water, w/w) with 250 mM buffer solution at a ratio of 9:1. Enzyme working solutions were prepared in water at a certain dose (showing signal within linear range as per dose response curve). All the incubations were carried out at 50° C. for 10 min following the same protocol as described for glucoamylase activity assay in Example 3. Enzyme activity at each pH was reported as relative activity compared to enzyme activity at optimum pH. The pH profile of AfGA1TR is shown in Table 3 and FIG. 3. AfGA1TR was found to have an optimum pH at about 5.0, and retain greater than 70% of maximum activity between pH 3.5 and 7.5.

TABLE 3
pH profiles for purified glucoamylases

| | Relative activity (%) | | |
|---|---|---|---|
| pH | AnGA | Native AfGA | AfGA1TR |
| 3 | 73.5 | 54.9 | 52.6 |
| 4 | 94.9 | 92.5 | 88.3 |
| 5 | 100 | 97.6 | 100 |
| 6 | 95.2 | 100 | 99.3 |
| 7 | 66.5 | 79.2 | 87.9 |
| 8 | 23.8 | 43 | 42.1 |
| 9 | 9.9 | 11 | 11.7 |
| 10 | 5.3 | 8.4 | 5.3 |

### Example 5

### Effect of Temperature on AfGA1TR Glucoamylase Activity

The effect of temperature (from 40° C. to 84° C.) on AfGA1TR activity was monitored using the ABTS assay protocol as described in Example 3. Substrate solutions were prepared by mixing 3.6 mL of soluble starch (1% in water, w/w) and 0.4 mL of 0.5 M buffer (pH 5.0 sodium acetate) into a 15-mL conical tube. Enzyme working solutions were prepared in water at a certain dose (showing signal within linear range as per dose response curve). Incubations were done at temperatures from 40° C. to 84° C., respectively, for 10 min following the same protocol as described for glucoamylase activity assay in Example 3. Enzyme activity at each temperature was reported as relative activity compared to enzyme activity at optimum temperature. The temperature profile of AfGA1TR is shown in Table 4 and FIG. 4. AfGA1TR was found to have an optimum temperature of 68° C., and retain greater than 70% of maximum activity between 56° C. and 74° C.

TABLE 4
Temperature profiles for glucoamylases.

| | Relative activity (%) | | |
|---|---|---|---|
| | | Native | |
| Temp (° C.) | AnGA | AfGA | AfGA1TR |
| 40 | 33.9 | 36.6 | 32.7 |
| 42.1 | 36 | 38.2 | 37.6 |
| 46.5 | 46.1 | 49.4 | 45 |
| 54 | 57.2 | 66.3 | 61.9 |
| 60 | 85.1 | 91.9 | 87.1 |
| 66.6 | 100 | 100 | 100 |
| 74.1 | 31.1 | 39.4 | 70.4 |
| 80.2 | 11.8 | 10.9 | 11.2 |
| 83.5 | 8.8 | 9.6 | 8.8 |

### Example 6

### AfGA1TR Product Profile Analysis

To assay the products of fungal glucoamylase catalysis of polysaccharides, the glucoamylases, AnGA (0.118 mg/gds starch) and AfGA1TR (0.118 mg/gds or 0.059 mg/gds), were incubated with 34% DS LIQUOZYME® Supra liquefied starch (CPI, Stockton, Calif.), at 60° C., pH 4.2 to 4.5 for 2 days. Pullulanase (PU) and acid-stable alpha-amylase from Aspergillus kawachii, GC626® (AkAA) were dosed along with purified AfGA1TR at 0.256 ASPU/gds and 0.35 SSU/gds, respectively. Samples were taken at different intervals of time and analyzed for sugar composition by HPLC.

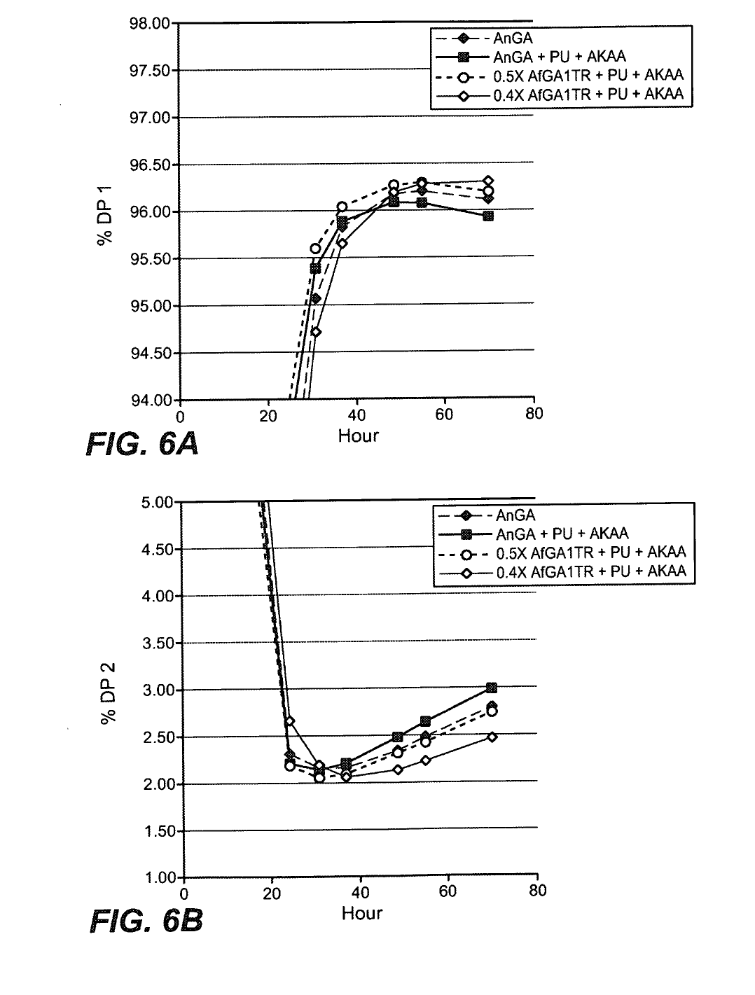

Table 5 shows the profile of oligosaccharides saccharified by AnGA/PU/AkAA and AfGA1TR/PU/AkAA at 100% and 50% the concentration of AnGA. (FIG. 6 depicts the profile of oligosaccharides saccharified by AnGA and AfGA1TR at 100%, 50% and 40% the concentration of AnGA, with and without PU and AkAA). Only oligosaccharides with DP1, DP2, DP3 and HS are shown. The numbers in Table 5 reflect the weight percentage of each DPn as a fraction of the total DP1, DP2, DP3, and HS.

TABLE 5
Product profile of fungal glucoamylases on liquefied starch.

| | | | | % | % | % | % |
|---|---|---|---|---|---|---|---|
| Flask | Enzymes | Dose:/gds | Hr | DP1 | DP2 | DP3 | HS |
| 1 | AnGA + | 0.118 mg + | 8 | 69.75 | 9.29 | 0.66 | 20.30 |
| | PU + GC626 | 0.256ASPU + | 24 | 93.40 | 2.21 | 0.71 | 3.68 |
| | | 0.35SSU | 31 | 95.40 | 2.15 | 0.67 | 1.79 |
| | | | 37 | 95.90 | 2.22 | 0.60 | 1.27 |
| | | | 48.5 | 96.10 | 2.48 | 0.53 | 0.89 |
| | | | 55 | 96.09 | 2.64 | 0.49 | 0.78 |
| | | | 70 | 95.95 | 2.99 | 0.45 | 0.62 |
| 2 | AfGA1TR + | 0.118 mg + | 8 | 86.34 | 2.35 | 0.47 | 10.84 |
| | PU + GC626 | 0.256ASPU + | 24 | 96.01 | 2.29 | 0.49 | 1.21 |
| | | 0.35SSU | 31 | 96.11 | 2.58 | 0.44 | 0.88 |
| | | | 37 | 96.03 | 2.84 | 0.42 | 0.71 |
| | | | 48.5 | 95.70 | 3.33 | 0.43 | 0.54 |
| | | | 55 | 95.52 | 3.56 | 0.44 | 0.48 |
| | | | 70 | 94.97 | 4.12 | 0.49 | 0.42 |
| 3 | AfGA2TR + | 0.059 mg + | 8 | 71.27 | 9.07 | 0.61 | 19.05 |
| | PU + GC626 | 0.256ASPU + | 24 | 93.80 | 2.19 | 0.72 | 3.29 |
| | | 0.35SSU | 31 | 95.61 | 2.06 | 0.68 | 1.65 |
| | | | 37 | 96.06 | 2.09 | 0.63 | 1.22 |
| | | | 48.5 | 96.29 | 2.31 | 0.54 | 0.86 |
| | | | 55 | 96.31 | 2.43 | 0.50 | 0.76 |
| | | | 70 | 96.21 | 2.74 | 0.45 | 0.59 |

Table 5 showed that AfGA1TR resulted in >95.5% DP1 in 24 hours, compared to AnGA which took 48.5 hours under an equal dose of protein. The data in Table 5 showed that AfGA1TR demonstrated an improved performance over AnGA at 50% dose equivalent based on protein (under the identical conditions of complimentary enzymes dosage).

### Example 7

### Comparison of DP2 Levels

DP2 level from AfGA1TR treated liquefied starch was compared to the one from AnGA treated liquefied starch based on the same DP1 level (96%). The comparison showed a statistically significant reduction in DP2 level at equal DP1 level approx. by 0.2%, possibly due to lower glucoamylase dose. Reversion reaction by AnGA and AfGA1TR (as the triple blend) was measured by calculating isomaltose/maltose ratio through ion chromatography.

TABLE 6
Product profile of fungal glucoamylases on liquefied starch.

| | | % of the total | | % of the total DP2 | | | |
|---|---|---|---|---|---|---|---|
| | | composition after | | after glucoamylase | | reversion | |
| | | glucoamylase reaction | | reaction | | reaction | |
| | | % | % | % | % | Ratio | |
| | Hour | DP1 | DP2 | IsoMaltose | Maltose | (IsoM/M) | ΔRatio |
| AnGA | 48 | 96.08 | 2.26 | 55.6 | 44.4 | 1.25 | |
| | 70 | 95.91 | 2.77 | 64.9 | 35.1 | 1.85 | |
| AfGA1TR | 48 | 96.16 | 2.13 | 54.8 | 45.2 | 1.21 | 0.04 |
| | 70 | 96.10 | 2.60 | 63.4 | 36.6 | 1.73 | 0.13 |

Δ Ratio = [AnGA ratio − AfGA1TR ratio] at 48 and 70 hours

Table 6 shows that for both glucoamylases isomaltose is accumulating over time according to increasing ratio of Isomaltose:Maltose. However, isomaltose formation appears to be slightly lower with AfGA1TR because the difference of ratio between AnGA and AfGA1TR is increased from 48 hours to 70 hours, which may support the lower reversion reaction by AfGA1TR.

### Example 8

### Titration of AkAA

To assay the products of fungal glucoamylase catalysis of polysaccharides using varied doses of accessory alpha-amylase, AfGA1TR (0.059 mg/gds) and Pullulanase (PU, OPTIMAX® L-1000)(0.256 ASPU/gds) were incubated with different concentrations of acid-stable alpha-amylase, GC626® (AkAA) from Aspergillus kawachi. AkAA was added at 0 to 0.3 SSU/ds in increments of 0.1 SSU and 34% DS LIQUOZYME® Supra liquefied starch (CPI, Stockton, Calif.), at 60° C., pH 4.5 for 2 days. Pullulanase (PU) and GC626® (AkAA) were dosed along with purified AfGA1TR at 0.256 ASPU/gds and 0.35 SSU/gds, respectively. Samples were taken at different intervals of time and analyzed for sugar composition by HPLC.

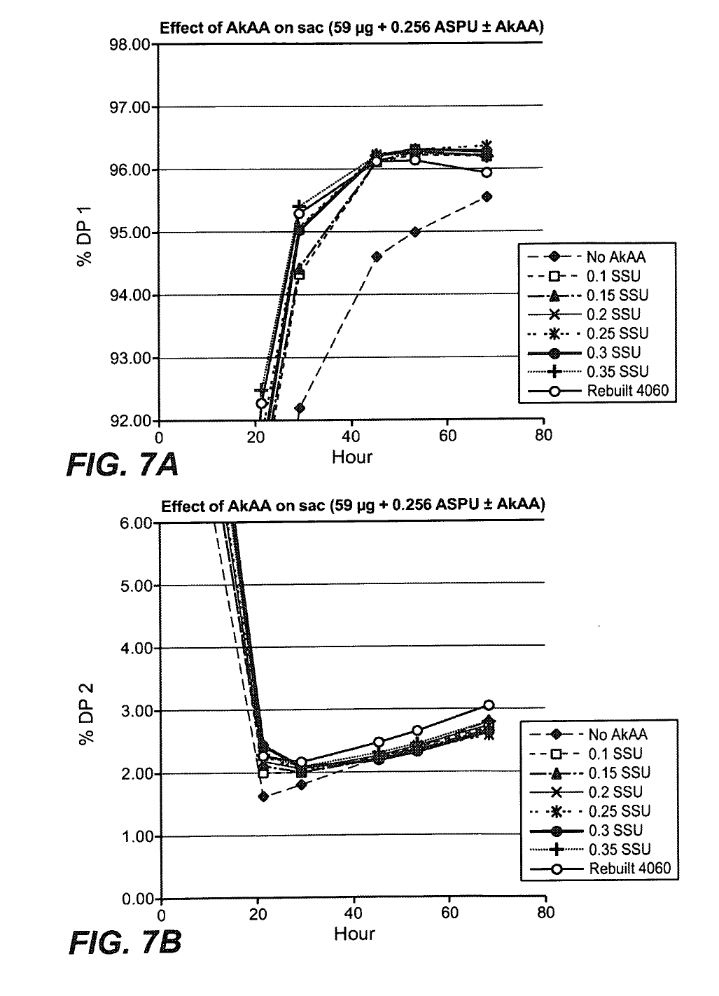

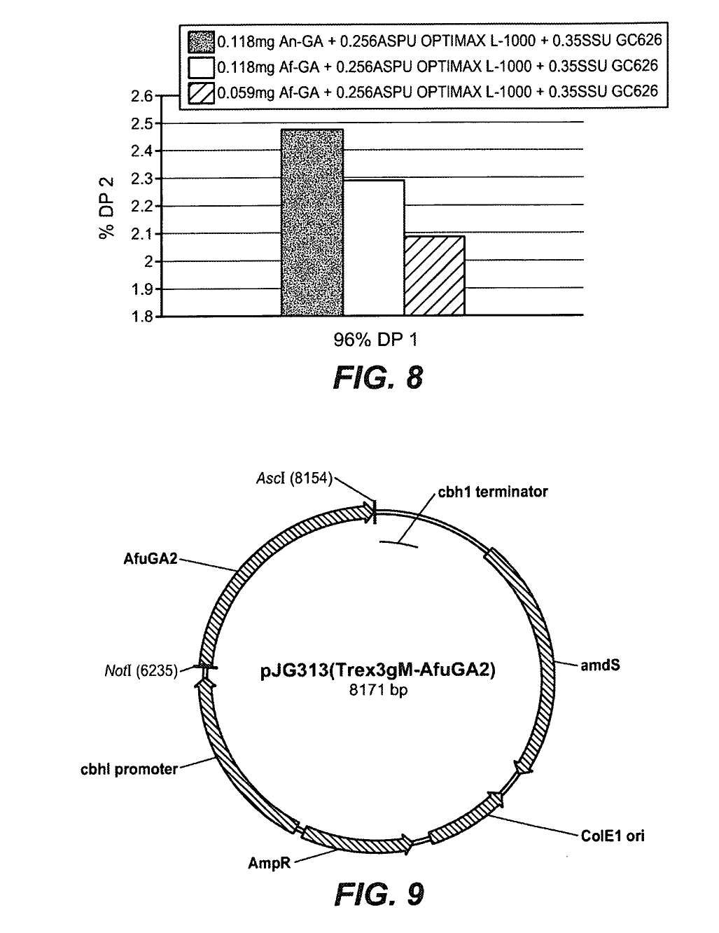

Table 7 and FIG. 7 disclose profiles of oligosaccharides saccharified by AfGA1TR, PU and varying doses of AkAA. Only oligosaccharides with DP1, DP2, DP3 and HS are shown. The numbers in Table 7 reflect the weight percentage of each DPn as a fraction of the total DP1, DP2, DP3, and HS.

TABLE 7
Effect of AkAA during saccharification
enzyme liquefied starch by AfGA1TR.

| | AkAA | | | | | |
|---|---|---|---|---|---|---|
| Flask | (GC626 ®)dose: /gds | Hr | % DP1 | % DP2 | % DP3 | % HS |
| 1 | 0 | 6 | 64.43 | 8.51 | 0.68 | 26.37 |
| | | 21 | 88.93 | 1.60 | 0.46 | 9.01 |
| | | 29 | 92.19 | 1.80 | 0.45 | 5.56 |
| | | 45 | 94.60 | 2.22 | 0.40 | 2.78 |
| | | 53 | 94.99 | 2.40 | 0.38 | 2.23 |
| | | 68 | 95.55 | 2.77 | 0.37 | 1.30 |
| 2 | 0.1 SSU | 6 | 63.10 | 10.00 | 0.77 | 26.12 |
| | | 21 | 91.07 | 2.01 | 0.59 | 6.33 |
| | | 29 | 94.33 | 2.01 | 0.58 | 3.09 |
| | | 45 | 96.13 | 2.23 | 0.49 | 1.15 |
| | | 53 | 96.24 | 2.39 | 0.46 | 0.91 |
| | | 68 | 96.20 | 2.72 | 0.42 | 0.66 |
| 3 | 0.2 SSU | 6 | 63.66 | 10.47 | 0.92 | 24.96 |
| | | 21 | 91.75 | 2.18 | 0.65 | 5.43 |
| | | 29 | 95.03 | 2.05 | 0.64 | 2.28 |
| | | 45 | 96.19 | 2.25 | 0.52 | 1.03 |
| | | 53 | 96.28 | 2.41 | 0.48 | 0.83 |
| | | 68 | 96.22 | 2.73 | 0.43 | 0.62 |
| 4 | 0.3 SSU | 6 | 59.97 | 11.76 | 1.74 | 26.53 |
| | | 21 | 91.52 | 2.44 | 0.71 | 5.33 |
| | | 29 | 95.03 | 2.08 | 0.70 | 2.19 |
| | | 45 | 96.21 | 2.20 | 0.57 | 1.01 |
| | | 53 | 96.31 | 2.34 | 0.52 | 0.83 |
| | | 68 | 96.27 | 2.64 | 0.46 | 0.63 |

Table 7 shows that AfGA1TR was able to reach >96% DP1 in 45 hours in presence of at least 0.1 SSU/gds, while lack of AkAA resulted in a statistically significantly reduced rate of saccharification. The result indicates that a significant increase in the final glucose yield was achieved by the addition of AkAA during saccharification of enzyme liquefied starch by AfGA1TR.

### Example 9

### Solubilization and Hydrolysis of Granular Starch by an Enzyme Blend Containing Alpha-Amylase, AfGA1TR and Pullulanase

Granular corn starch slurry having 35% dry solid starch in distilled water was prepared and the pH was adjusted to pH 5.0 using NaOH. 10 AAU/gds of alpha-amylase (SPEZYME® XTRA) and purified protein of AfGA1TR were added at 0.047 mg/gds along with 0.15 ASPU/gds of pullulanase (OPTIMAX® L-1000) to the starch slurry. Then, the starch slurry was kept in a water bath maintained at 60° C. with constant stirring. An aliquot was withdrawn at different time intervals and centrifuged. The clear supernatant was used for refractive index (RI) to calculate percent solubilization and analyzed for sugar composition by HPLC.

TABLE 8
Product profile of fungal glucoamylase-on starch during liquefaction.

| Enzymes | Dose:/gds | | Hr | % Solubility | % DP1 | % DP2 | % DP3 | % HS |
|---|---|---|---|---|---|---|---|---|
| SPEZYME XTRA | 10 | AAU | 5 | 52.5% | 73.05 | 13.20 | 1.55 | 12.20 |
| | | | 20.5 | 72.5% | 93.00 | 2.38 | 1.86 | 2.76 |
| | | | 29 | 76.2% | 94.10 | 2.21 | 1.55 | 2.13 |
| OPTIMAX L-1000 | 0.15 | ASPU | 44.5 | 82.0% | 95.14 | 2.16 | 1.16 | 1.54 |
| | | | 52 | 83.5% | 95.43 | 2.20 | 1.02 | 1.36 |
| AfGA1TR | 0.047 | mg | 68 | 86.1% | 95.75 | 2.36 | 0.81 | 1.08 |

Table 8 shows that AfGA1TR was able to reach >95.5% DP1 in 68 hours using granular starch in presence of alpha-amylase and PU, where granular starch was 86% solubilized.

### Example 10

### Effect of Residual Alpha-Amylase Activity on DP3 Level

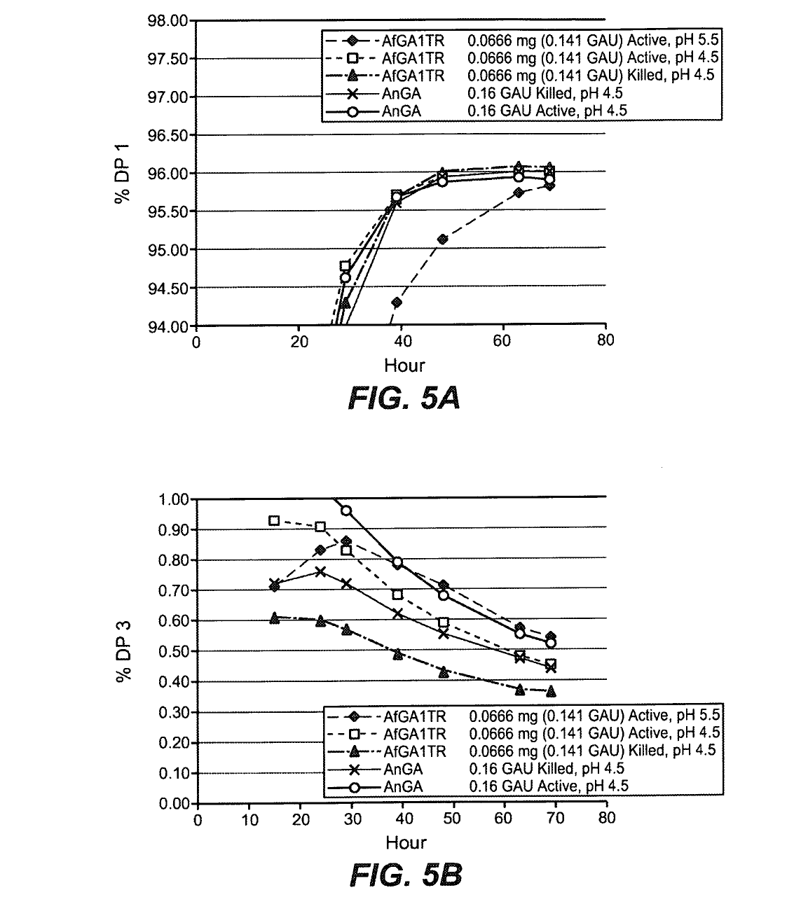

The effect of single pH (5.5) and the effect of residual alpha-amylase activity at pH 4.5 on both DP1 and DP3 with AfGA1TR by adding 0.066 KG/MTds of LIQUOZYME® Supra (NZ) back to alpha-killed starch liquefact was analyzed. 0.066 mg/gds of AfGA1TR was blended with 0.25 ASPU/gds of OPTIMAX® L-1000 (pullulanase) and 0.1 SS U/gds of AkAA. Table 9 and FIG. 5 disclose profiles of oligosaccharides saccharified by AfGA1TR and OPTIMAX® 4060 VHP (an AnGA/pullanase blend).

TABLE 9
Product profile of fungal glucoamylase on liquefied starch.

| Sugar composition at 48 hours | % DP1 | % DP2 | % DP3 | % HS |
|---|---|---|---|---|
| AfGA1TR triple 0.066 mg | 95.12 | 1.99 | 0.71 | 2.19 |
| (0.141GAU), Active, pH 5.5 | | | | |
| AfGA1TR triple 0.066 mg | 95.92 | 2.19 | 0.59 | 1.30 |
| (0.141GAU), Active, pH 4.5 | | | | |
| AfGA1TR triple 0.066 mg | 96.00 | 2.26 | 0.43 | 1.31 |
| (0.141GAU), Killed, pH 4.5 | | | | |
| OPTIMAX ® 4060 VHP | 95.93 | 2.27 | 0.55 | 1.25 |
| 0.16GAU, Killed, pH 4.5 | | | | |
| OPTIMAX ® 4060 VHP | 95.86 | 2.27 | 0.68 | 1.18 |
| 0.16GAU, Active, pH 4.5 | | | | |

AfGA1TR triple blend showed significant loss in the rate of saccharification at pH 5.5 compared to pH 4.5 possibly due to unfavorable pH to OPTIMAX® L-1000 but was able to achieve >95.5% DP1 in 48 hours. Both AfGA1TR and OPTIMAX® 4060 VHP were a bit negatively affected by residual alpha-amylase activity to maximize glucose yield because of higher DP3 as expected. In the case of alpha-amylase killed liquefact, AfGA1TR resulted in significantly lower DP3 than OPTIMAX® 4060 VHP by 0.1%. Levels of AfGA1TR with alpha-amylase active liquefact were as low as the one of OPTIMAX® 4060 VHP with alpha-“killed” liquefact.

### Example 11

### Comparison of AfGA1TR with Wild-Type Aspergillus fumigatus Glucoamylase

Starch liquefact was prepared to have 34% dry solids by diluting with water and the saccharification was carried out using the 2 different glucoamylases; 1) AfGA1TR at 0.067 mg/gds starch and 2) purified protein of wild-type AfGA (expressed in Aspergillus fumigatus) from GLUCOTEAM DB (Nagase Co. & Ltd., Japan) at 0.065 mg/gds at pH 4.4 and 60° C. In addition, pullulanase (PU) and acid-stable alpha-amylase, GC626® (AkAA) at 0.14 ASPU/gds and 0.9 SSU/gds, respectively, were dosed along with each glucoamylase. Samples were taken at different intervals of time and analyzed for sugar composition by HPLC.

Table 10 showed that AfGA1TR resulted in >95.5% DP1 in 48 hours, whereas commercial Aspergillus fumigatus took longer saccharification time. Both glucoamylases were able to reach >96% DP1 with DP2 less than 3%.

TABLE 10
Product profile of fungal glucoamylase blends on liquefied starch.

| | | | | % | % | % | % |
|---|---|---|---|---|---|---|---|
| Enzymes | Dose:/gds | | Hr | DP1 | DP2 | DP3 | HS |
| AFGA1TR | 0.067 | mg | 15 | 86.62 | 4.60 | 0.76 | 8.02 |
| | | | 26 | 94.78 | 2.11 | 0.77 | 2.34 |
| OPTIMAX L-1000 | 0.14 | ASPU | 39 | 96.12 | 2.07 | 0.55 | 1.26 |
| | | | 48 | 96.27 | 2.26 | 0.45 | 1.02 |
| | | | 63 | 96.22 | 2.61 | 0.34 | 0.83 |
| GC626 ® | 0.9 | SSU | 71 | 96.16 | 2.77 | 0.31 | 0.76 |
| Wild-type AfGA | 0.065 | mg | 16 | 75.43 | 10.45 | 0.84 | 13.28 |
| | | | 24 | 87.33 | 4.87 | 0.93 | 6.87 |
| OPTIMAX L-1000 | 0.14 | ASPU | 40 | 95.11 | 2.05 | 0.89 | 1.95 |
| | | | 48 | 95.81 | 1.93 | 0.79 | 1.47 |
| | | | 64 | 96.16 | 2.09 | 0.61 | 1.14 |
| GC626 ® | 0.9 | SSU | 72 | 96.21 | 2.19 | 0.55 | 1.05 |

### Example 12

### Solubilization and Hydrolysis of Granular Starch by Enzyme Blend Containing Alpha-Amylase, Pullulanase and Aspergillus fumigatus GAs

In a typical example, granular corn starch slurry having 35% dry solid starch in distilled water was prepared and the pH was adjusted to pH 5.0 using sodium hydroxide. Ten AAU/gds of SPEZYME® XTRA and purified protein of AfGA1TR or wild-type Aspergillus fumigatus GA (AfGA) from GLUCOTEAM DB (Nagase Co. & Ltd., Japan) were added along with 0.15 ASPU/gds of OPTIMAX® L-1000 to the starch slurry. Then, the starch slurry was kept in a water bath maintained at 60° C. with constant stirring. An aliquot was withdrawn at different time intervals and centrifuged. The clear supernatant was used for refractive index (RI) to calculate percent solubilization and analyzed for sugar composition by HPLC. Table 11 shows that both Aspergillus fumigatus glucoamylases were able to reach >95.5% DP1 by 68 hours using granular starch in presence of alpha-amylase and PU, where granular starch was >86% solubilized.

TABLE 11
Effect of alpha-amylase and pullulanase on granular starch solubilization

| Enzymes | Dose:/gds | | Hr | % Solubility | % DP1 | % DP2 | % DP3 | % HS |
|---|---|---|---|---|---|---|---|---|
| SPEZYME XTRA | 10 | AAU | 5 | 52.5% | 73.05 | 13.20 | 1.55 | 12.20 |
| | | | 20.5 | 72.5% | 93.00 | 2.38 | 1.86 | 2.76 |
| OPTIMAX L-1000 | 0.15 | ASPU | 29 | 76.2% | 94.10 | 2.21 | 1.55 | 2.13 |
| | | | 44.5 | 82.0% | 95.14 | 2.16 | 1.16 | 1.54 |
| | | | 52 | 83.5% | 95.43 | 2.20 | 1.02 | 1.36 |
| AfGA1TR | 0.047 | mg | 68 | 86.1% | 95.75 | 2.36 | 0.81 | 1.08 |
| SPEZYME XTRA | 10 | AAU | 15 | 72.3% | 87.15 | 4.92 | 2.25 | 5.68 |
| | | | 25 | 78.7% | 91.93 | 2.68 | 2.15 | 3.24 |
| OPTIMAX L-1000 | 0.14 | ASPU | 39 | 86.0% | 94.24 | 2.00 | 1.70 | 2.06 |
| | | | 50 | 87.3% | 94.84 | 1.96 | 1.45 | 1.75 |
| | | | 64 | 91.5% | 95.42 | 2.04 | 1.15 | 1.39 |
| Wild-type AfGA | 0.075 | mg | 73 | 92.1% | 95.62 | 2.12 | 1.01 | 1.25 |

### Example 13

### Expression and Purification of AfGA2TR

The nucleic acid sequence for the AfGA2 gene (NCBI Reference Sequence DS499595 from 145382 to 147441) was mined from Aspergillus fumigatus A1163, and the amino acid sequence of the hypothetical protein encoded by the AfGA2 gene was found in the NCBI Databases (NCBI Accession No. EDP53734). The nucleotide sequence of the AfGA2 gene from Aspergillus fumigatus A1163 was optimized and synthesized by Generay (Generay Biotech Co., Ltd, Shanghai, China).

The DNA sequence of AfGA2 was optimized for its expression in Trichoderma reesei, then synthesized and inserted into the pTrex3gM expression vector (described in U.S. Published Application 2011/0136197 A1) by Generay (Generay Biotech Co., Ltd, Shanghai, China), resulting in pJG313 (FIG. 9).

The plasmid pJG313 was transformed into a quad-deleted Trichoderma reesei strain (described in WO 05/001036) using biolistic method (Te'o et al., J. Microbiol. Methods 51:393-99, 2002). Transformants were selected on a medium containing acetamide as a sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies (about 50-100) appeared in about 1 week. After growth on acetamide plates, transformants were picked and transferred individually to acetamide agar plates. After 5 days of growth on acetamide plates, transformants displaying stable morphology were inoculated into 200 μl Glucose/Sophorose defined media in 96-well microtiter plates. The microtiter plate was incubated in an oxygen growth chamber at 28° C. for 5 days. Supernatants from these cultures were used to confirm the protein expression by SDS-PAGE analysis and assay for enzyme activity. The stable strains were subsequently grown in a 7 L fermenter in a defined medium containing 60% glucose-sophorose feed. Glucose/Sophorose defined medium (per liter) consists of (NH₄)₂SO₄ 5 g, PIPPS buffer 33 g, Casamino Acids 9 g, KH₂PO₄ 4.5 g, CaCl₂ (anhydrous) 1 g, MgSO₄.7H₂O 1 g, pH to 5.5 adjusted with 50% NaOH with Milli-Q H₂O to bring to 966.5 mL. After sterilization, the following were added: 26 mL 60% Glucose/Sophrose, and 400×T. reesei Trace Metals 2.5 mL.

The protein, AfGA2TR, was purified via two steps of chromatography. Ammonium sulfate was added to 600 mL fermentation broth until the final concentration of ammonium sulfate reaches 1 M. The sample was loaded onto a 50 mL hydrophobic interaction chromatography Phenyl HP column pre-equilibrated with 20 mM sodium phosphate pH 7.0 containing 1 M Ammonium sulfate (buffer A). The column was washed with a linear salt gradient from 1 to 0 M ammonium sulfate. The active fractions were pooled and applied to affinity chromatography. The sample after hydrophobic interaction chromatography was exchanged into 25 mM pH 4.3 sodium acetate buffer (buffer B) and loaded onto β-cyclodextrin coupled Sepharose 6B column pre-equilibrated with buffer B. The target protein was eluted by 25 mM pH 4.3 sodium acetate with 10 mM α-cyclodextrin (Buffer C). The eluant was concentrated by using a 10K Amicon Ultra-15 device. The final product was above 98% pure and stored in 40% glycerol at −80° C. for further studies.

### Example 14

### Glucoamylase Activity of AfGA2TR

AfGA2TR belongs to the glycosyl hydrolase 15 family (GH15, CAZy number). The glucoamylase activity of AfGA2TR was measured using 1% w/w soluble starch (Sigma S9765) as a substrate. The assay was performed in 50 mM sodium acetate buffer pH 5.0 at 50° C. for 10 minutes. The rate of glucose release was measured using the glucose oxidase/peroxidase (GOX/HRP) technique disclosed in Example 3. Glucose was quantified as the rate of oxidation of 2,2′-azino-bis 3-ethylbenzothiazoline-6-sulfonic acid (ABTS) via excess coupled GOX/HRP enzymes. The enzyme activity was calculated based on a glucose standard curve. In this assay, one glucoamylase unit is defined as the amount of enzyme required to generate 1 μmol of glucose per minute under the conditions of the assay. The specific activity towards soluble starch of purified AfGA2TR was determined to be 214 units/mg using the above method.

### Example 15

### pH Profile of AfGA2TR

The effect of pH (from 3.0 to 10.0) on AfGA2TR activity was monitored by following the ABTS assay protocol as described in Example 3. Buffer working solutions consisted of the combination of glycine/sodium acetate/HEPES (250 mM), with pH variation from 3.0 to 10.0. Substrate solutions were prepared by mixing soluble starch (1% in water, w/w) with 250 mM buffer solution at a ratio of 9:1. Enzyme working solutions were prepared in water at a certain dose (showing signal within linear range as per dose response curve). All the incubations were done at 50° C. for 10 min using the same protocol as described for glucoamylase activity assay in Example 3. Enzyme activity at each pH was reported as relative activity towards enzyme activity at optimum pH. The pH profile of AfGA2TR is shown in FIG. 10. AfGA2TR was found to have an optimum pH at about 5.3, and retain greater than 70% of maximum activity between pH 3.3 and 7.3.

### Example 16

### Temperature Profile of AfGA2TR

The effect of temperature (from 40° C. to 84° C.) on AfGA2TR activity was monitored by following the ABTS assay protocol as described in Example 3. Substrate solutions were prepared by mixing 9 mL of soluble starch (1% in water, w/w) and 1 mL of 0.5 M buffer (pH 5.0 sodium acetate) into a 15-mL conical tube. Enzyme working solutions were prepared in water at a certain dose (showing signal within linear range as per dose response curve). Incubations were carried out at temperatures from 40° C. to 84° C., respectively, for 10 min. After incubation, the activities were determined following the same protocol as described for glucoamylase activity assay in Example 3. The activity was reported as relative activity towards the enzyme activity at optimum temperature. The temperature profile of AfGA2TR is shown in FIG. 11. AfGA2TR was found to have an optimum temperature of 69° C., and retain greater than 70% of maximum activity between 61° C. and 74° C.

### Example 17

### Thermostability of AfGA2TR

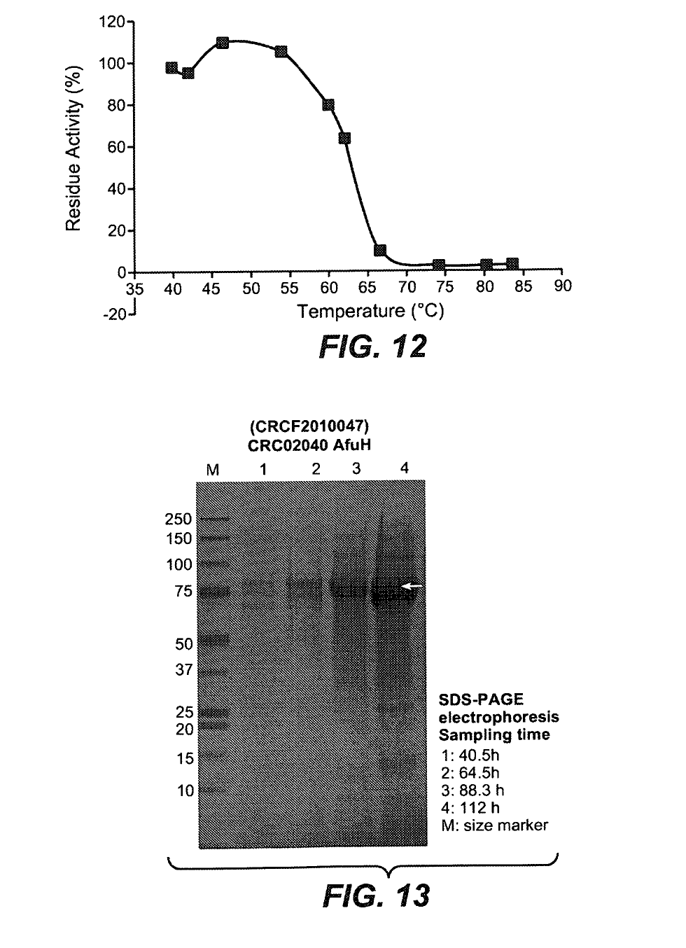

The thermostability of AfGA2TR was determined in 50 mM sodium acetate buffer pH 5.0. The enzyme was incubated at desired temperature for 2 hours in a PCR machine prior to addition into substrate. The remaining activity of the samples was measured as described in Example 3. The activity of the sample kept on ice was defined as 100% activity. As shown in FIG. 12, at temperature lower than 63° C., AfGA2TR retained over 50% activity during a 2-hour incubation period.

### Example 18

### Comparison of AfGA1TR with AfGA2TR

Starch liquefact was prepared to have 34% dry solids by diluting with water and the saccharification was carried out using the 2 different glucoamylases; 1) AfGA1TR at 0.06 mg/gds starch and 2) purified protein of AfGA2TR at 0.06 mg/gds at pH 4.4 and 60° C. In addition, pullulanase (OPTIMAX® L-1000) and acid-stable alpha-amylase, GC626® (AkAA) at 0.14 ASPU/gds and 0.9 SSU/gds, respectively, were dosed along with each glucoamylase to enhance glucose production. Samples were taken at different intervals of time and analyzed for sugar composition by HPLC.

TABLE 12
Product profile of AfGA1TR and AfGA2TR
blends on liquefied starch.

| | | | | % | % | % | % |
|---|---|---|---|---|---|---|---|
| Enzymes | Dose:/gds | | Hr | DP1 | DP2 | DP3 | HS |
| AFGA1TR | 0.06 | mg | 16 | 82.99 | 6.76 | 1.14 | 9.11 |
| | | | 24 | 91.65 | 3.30 | 1.21 | 3.84 |
| | | | 40 | 95.32 | 2.33 | 0.93 | 1.42 |
| OPTIMAX ® L-1000 | 0.14 | ASPU | 48 | 95.66 | 2.39 | 0.79 | 1.16 |
| | | | 64 | 95.73 | 2.71 | 0.64 | 0.92 |
| GC626 ® | 0.9 | SSU | 72 | 95.64 | 2.87 | 0.62 | 0.87 |
| AfGA2TR | 0.06 | mg | 16 | 86.40 | 4.32 | 1.05 | 8.23 |
| | | | 24 | 93.34 | 2.34 | 1.05 | 3.27 |
| OPTIMAX ® L-1000 | 0.14 | ASPU | 40 | 95.68 | 2.17 | 0.78 | 1.37 |
| | | | 48 | 95.91 | 2.28 | 0.67 | 1.14 |
| | | | 64 | 95.93 | 2.62 | 0.55 | 0.90 |
| GC626 ® | 0.9 | SSU | 72 | 95.88 | 2.77 | 0.51 | 0.84 |

Table 12 showed that both glucoamylases resulted in >95.5% DP1 in 48 hours with a slightly faster saccharification using AfGA2TR.

### Example 19

### Simultaneous Liquefaction and Saccharification of Starch with AfGA1TR

An aqueous slurry containing 32% ds corn starch was prepared using standard methods known in the art. The pH was adjusted to 5.0 using sodium hydroxide. A thermostable liquefying alpha-amylase, SPEZYME® Xtra (DuPont Industrial Bioscience), was added at 1.2 kg./MT ds corn starch. AfGA1TR was added at 1.0, 2.0, and 5 mg of protein grams ds starch. The reaction mixture was then placed in a water bath maintained at 80° C. The reaction mixture was constantly mixed during incubation and samples were taken at different intervals of time for total sugar composition and percent starch solubilized (Table 13).

TABLE 13
Simultaneous Liquefaction and Saccharification of
Starch Above Starch Gelatinization Temperature.

| | SLS | | | | | | |
|---|---|---|---|---|---|---|---|
| GAU/ | Time, | % | Supernatant | % composition of the sugar | | | |
| gds | Hour | Solubility | DS (%) | DP1 | DP2 | DP3 | DP4+ |
| 0.5 | 23 | 93.7 | 36.0 | 91.00 | 2.47 | 1.30 | 5.23 |
| | 31 | 94.3 | 36.2 | 90.94 | 2.49 | 1.31 | 5.26 |
| 1.0 | 23 | 94.0 | 36.6 | 94.21 | 2.82 | 0.81 | 2.16 |
| | 31 | 95.4 | 37.0 | 94.15 | 2.83 | 0.82 | 2.20 |

In this example, a syrup containing over 90% glucose was generated by a simultaneous liquefaction and saccharification process conducted 8 degrees above the gelatinization temperature of corn starch. Specifically, the results in Table 13 demonstrate that that AfGA1TR was able to produce >90% DP1 at 80° C. with >93% starch solubilization, which is a significant improvement from previously suggested process for granular starch hydrolysis below starch gelatinization temperature in a given time.

Although the compositions and methods of making and using has been described in detail with reference to examples above, it is understood that various modifications can be made without departing from the spirit these compositions and methods, and would be readily known to the skilled artisan.

All cited patents and publications referred to in this application are herein incorporated by reference in their entirety for all purposes.

SEQUENCE LISTING

SEQ ID NO: 1-AfGA1 precursor
MPRLSYALCALSLGHAAIAAPQLSARATGSLDSWLGTETTVALNGILANIGADGAYAKSAKPGIIIASPS
TSEPDYYYTWTRDAALVTKVLVDLFRNGNLGLQKVITEYVNSQAYLQTVSNPSGGLASGGLAEPKYNVDM
TAFTGAWGRPQRDGPALRATALIDFGNWLIDNGYSSYAVNNIWPIVRNDLSYVSQYWSQSGFDLWEEVNS
MSFFTVAVQHRALVEGSTFAKRVGASCSWCDSQAPQILCYMQSFWTGSYINANTGGGRSGKDANTVLASI
HTFDPEAGCDDTTFQPCSPRALANHKVYTDSFRSVYAINSGIPQGAAVSAGRYPEDVYYNGNPWFLTTLA
AAEQLYDAIYQWKKIGSISITSTSLAFFKDIYSSAAVGTYASSTSTFTDIINAVKTYADGYVSIVQAHAM
NNGSLSEQFDKSSGLSLSARDLTWSYAAFLTANMRRNGVVPAPWGAASANSVPSSCSMGSATGTYSTATA
TSWPSTLTSGSPGSTTTVGTTTSTTSGTAAETACATPTAVAVTFNEIATTTYGENVYIVGSISELGNWDT
SKAVALSASKYTSSNNLWYVSVTLPAGTTFEYKYIRKESDGSIVWESDPNRSYTVPAACGVSTATENDTW
Q
SEQ ID NO: 2-AfGA2 precursor
MPRLSYALCALSLGHAAIAAPQLSARATGSLDSWLGTETTVALNGILANIGADGAYAKSAKPGIIIASPS
TSEPDYYYTWTRDAALVTKVLVDLFRNGNLGLQKVITEYVNSQAYLQTVSNPSGGLASGGLAEPKYNVDM
TAFTGAWGRPQRDGPALRATALIDFGNWLIDNGYSSYAVNNIWPIVRNDLSYVSQYWSQSGFDLWEEVNS
MSFFTVAVQHRALVEGSTFAKRVGASCSWCDSQAPQILCYMQSFWTGSYINANTGGGRSGKDANTVLASI
HTFDPEAGCDDTTFQPCSPRALANHKVYTDSFRSVYAINSGIPQGAAVSAGRYPEDVYYNGNPWFLTTLA
AAEQLYDAIYQWKKIGSISITSTSLAFFKDIYSSAAVGTYASSTSTFTDIINAVKTYADGYVSIVQAHAM
NNGSLSEQFDKSSGLSLSARDLTWSYAAFLTANMRRNGVVPAPWGAASANSVPSSCSMGSATGTYSTATA
TSWPSTLTSGSPGSTTTVGTTTSTTSGTATETACATPTAVAVTFNEIATTTYGENVYIVGSISELGNWDT
SKAVALSASKYTSSNNLWYVSVTLPAGTTFEYKYIRKESDGSIVWESDPNRSYTVPAACGVSTATENDTW
R
SEQ ID NO: 3-Nf_NRRL_181_GA
MPRLSYALCALSLGHAAIAAPQLSPRATGSLDSWLATESTVSLNGILANIGADGAYAKSAKPGIIIASPS
TSDPDYYYTWTRDAALVTKVLVDLFRNGNLGLQKVITEYVNSQAYLQTVSTPSGGLSSGGLAEPKYNVDM
TAFTGAWGRPQRDGPALRATALIDFGNWLIDNGYSSYAVNNIWPIVRNDLSYVSQYWSQSGFDLWEEVNS
MSFFTVAVQHRALVEGSTFAKRVGASCSWCDSQAPQILCYMQSFWTGSYINANTGGGRSGKDANTVLASI
HTFDPEAGCDDTTFQPCSPRALANHKVYTDSFRSVYAINSGIPQGVAVSAGRYPEDVYYNGNPWFLTTLA
AAEQLYDAIYQWKKIGSISITSTSLAFFKDIYSSVAVGTYASSSSTFTAIIDAVKTYADGYVSIVEAHAM
TNGSLSEQFDKSSGMSLSARDLTWSYAALLTANMRRNGVVPAPWGAASANSVPSSCSMGSATGTYSTATA
TSWPSTLTSGSPSDTTSGTTPGTTTTTSACTTPTSVAVTFDEIATTTYGENVYIIGSISQLGSWDTSKAV
PLSSSKYTSSNNLWYVTINLPAGTTFEYKYIRKESDGSIEWESDPNRSYTVPSACGVSTATEKDTWR
SEQ ID NO: 4-Ts_ATCC0_10500_GA
MTRLSSVLCALAALGQTALAAPGLSPRASTSLDAWLATETTVSLSGILANIGADGAYSKSAKPGVVIASP
STDNPNYYYTWTRDSALTLKVLIDLFRNGNLGLQTVIEEYVNAQAYLQTVSNPSGDLSSGAGLAEPKFNV
DMSAFTGSWGRPQRDGPALRAIALIDFGNWLIENGYTSLAANNIWPIVRNDLSYVAQYWSQSGFDLWEEV
NSMSFFTVANQHRSLVEGSTFAAKVGASCSWCDSQAPQILCYMQTFWTGSYMNANTGGGRSGKDANTVLT
SIATFDPEATCDDVTFQPCSPRALANHKVYTDSFRSVYGLNSGIAEGVAVAVGRYPEDSYYNGNPWFLSN
LAAAEQLYDAIYQWNKIGSITITSTSLAFFKDVYSSAAVGTYASGSSAFTSIINAVKTYADGYISVVQSH
AMNNGSLSEQFDKNTGAELSARDLTWSYAALLTANMRRNGVVPPSWGAASATSIPSSCTTGSAIGTYSTP
TATSWPSTLTSGTGSPGSTTSATGSVSTSVSATTTSAGSCTTPTSVAVTFDEIATTSYGENVYIVGSISQ
LGSWNTANAIALSASKYTTSNNLWYVTINLPAGTTFQYKYIRKESDGTVKWESDPNRSYTVPSACGVSTA
TENDTWR
SEQ ID NO: 5-Pm_ATCC_18224_GA
MTFSRLSSSVLCALAALGHNALAAPQFSPRATVGLDAWLASETTFSLNGILANIGSSGAYSASAKPGVVI
ASPSTNNPNYYYTWTRDSALTLKVLIDLFGNGNLSLQTVIEEYINAQAYLQTVSNPSGDLSSGAGLAEPK
YNVDMSPFTGGWGRPQRDGPALRAIALIEFGNWLIDNGYSSYAVNNIWPIVRNDLSYVSQYWSQSGFDLW
EEVNSMSFFTVANQHRALVQGSTFAARVGASCSWCDSQAPQILCYMQTFWTGSYINANTGGGRSGKDSNT
VLTTIHTFDPEATCDDVTFQPCSPRALANHKVYTDSFRSIYGVNSGIAQGVAVSVGRYPEDSYYGGNPWF
LSNLAAAEQLYDAIYQWNKIGSITITSTSLAFFKDVYSSAAVGTYASGSTAFTSIISAVKTYADGYVSIV
QGHAAANGSLSEQFDRNSGVEISARDLTWSYAALLTANLRRNGVMPPSWGAASANSVPSSCSMGSATGTY
STPTATAWPSTLTSATGIPVTTSATASVTKATSATSTTTSATTCTTPTSVAVTFDEIATTTYGENVFIVG
SISQLGSWDTSKAIALSASQYTSSNHLWFATLSLPAGTTFQYKYIRKESNGSIVWESDPNRSYTVPSGCG
VSTATENDTWR
SEQ ID NO: 6-An_FGSC_A4_GA
MPTTILKITLFPLIDSIFSVQLSPVRIAMLTLSKVLPVLALSHAVAAAPQLSARATASLNTWLSTEASFA
LDGILTNIGANGAYAKTAKAGADYYTWTRDAALTVKVLVDLFHNGDLSLQTILEEYTNSQAYLQTVSNPS
GGLASGGLAEPKFYVDMTAFTGSWGRPQRDGPALRATTLIGFGNWLIDNGYSSYASNNIWPIVRNDLTYV
AQYWSKSGYDLWEEVNSMSFFTVAVQHRALVEGSTFAHRVGASCPWCDSQAPQILCYMQNFWTGSYINAN
TGGGRSGKDANTVLASIHTFDPDAACDDITFQPCSSRALANHKVYTDSFRSVYSLNTGIAQGVAVAAGRY
PEDSYYNGNPWFLTTLAAAEQLYDAIYQWQKARSISITSTSLAFFKDIYSSAAVGTYASGSSAFTAIIDA
VKTYADGYVSIVKAHAMANGSLSEQFDKTYGTCVSARDLTWSYAALLTASMRRNGVVPPSWDAASANTLP
SSCSTGSATGTYSTATVTTWPSTLTSGSASATTTIMATSTATSSSTTTSTTTACTTPSTVAVTFNVIATT
TYGENVYIVGSISQLGNWDTGSAVALSASKNTSSNNLWYVDINLPGGTAFEYKYIRKETDGSIVWESDPN
RSYTVPSSCGVSTATESDTWRCTLETQSVRN
SEQ ID NO: 7-AfGA1 and AfGA2 CBM
FNEIATTTYGENVYIVGSISELGNWDTSKAVALSASKYTSSNNLWYVSVTLPAGTTFEYKYIRKESDGSI
VWESDPNRSYTVPAACGVSTATENDTW
SEQ ID NO: 8-AfGA1 gene of pTrex3gM-AfGA1
ATGCCTCGCCTTTCCTACGCGCTCTGTGCGCTGTCTCTCGGGCATGCTGCTATTGCAGCTCCTCAGTTAT
CCGCTCGTGCTACCGGCAGCTTGGACTCCTGGTTGGGTACTGAGACCACCGTTGCGCTCAATGGTATTCT
GGCCAACATCGGTGCCGACGGTGCTTATGCGAAGAGCGCTAAGCCTGGCATAATCATTGCCAGTCCGAGC
ACCAGCGAACCAGACTGTGAGAACCTTCCTGAACTGGCCCTGTCCGGCAGTCATTGACCTCGGTAGACTA
CTATACCTGGACGAGAGATGCTGCTCTCGTCACGAAAGTCCTGGTCGACCTCTTCCGCAACGGCAACCTG
GGTCTGCAGAAAGTCATTACCGAATACGTCAACTCTCAGGCGTACTTGCAGACCGTGTCTAATCCGTCGG
GTGGTCTTGCGAGCGGAGGTCTCGCGGAGCCTAAGTACAACGTCGACATGACGGCCTTTACCGGAGCCTG
GGGTCGTCCTCAGCGTGATGGTCCGGCTCTGCGGGCCACCGCCCTCATCGACTTTGGCAACTGGCTGATT
GTATGTTCTCCATACGAGCCCCAGGAAGCGTTGCTGACGTCTACAGGACAACGGCTACTCCAGCTATGCT
GTCAACAACATCTGGCCCATTGTGCGCAACGACTTGTCCTACGTTTCTCAGTACTGGAGCCAGAGTGGCT
TTGGTGAGTCCCGACTCTCTGGAAGTTTACAACGTGCATCGATTACTGACAATTGAGATTCTACGTGACA
GATCTCTGGGAAGAAGTCAACTCCATGTCCTTCTTCACCGTCGCTGTCCAGCACCGTGCCCTCGTGGAGG
GAAGCACGTTCGCTAAACGGGTGGGAGCGTCGTGCTCGTGGTGTGACTCGCAGGCCCCCCAGATCCTCTG
CTACATGCAGAGTTTCTGGACTGGCTCGTATATCAACGCCAACACCGGTGGTGGCCGGTCCGGCAAGGAT
GCCAACACCGTCCTCGCCAGCATCCATACCTTCGACCCCGAAGCCGGCTGCGACGATACTACTTTCCAGC
CCTGCTCTCCTCGGGCCCTTGCCAACCACAAGGTGTACACCGATTCGTTCCGCTCGGTCTACGCGATCAA
CTCCGGCATCCCACAGGGCGCTGCCGTTTCCGCTGGCCGCTACCCCGAGGACGTCTACTACAACGGCAAC
CCTTGGTTCCTCACCACCCTCGCCGCTGCCGAGCAGCTCTACGACGCTATCTACCAGTGGAAGAAGATCG
GTTCCATCAGCATCACCAGCACCTCCCTCGCCTTCTTCAAGGACATCTACAGCTCCGCCGCGGTCGGCAC
CTACGCCTCTAGCACCTCCACCTTCACGGACATCATCAACGCGGTCAAGACCTACGCAGACGGCTACGTG
AGCATCGTCCAGGCACACGCCATGAACAACGGCTCCCTTTCGGAGCAATTCGACAAGTCCTCTGGGCTGT
CCCTCTCCGCCCGCGATCTGACCTGGTCCTACGCCGCTTTCCTCACCGCCAACATGCGTCGTAACGGCGT
GGTGCCTGCCCCCTGGGGCGCCGCCTCCGCCAACTCCGTCCCCTCGTCTTGCTCCATGGGCTCGGCCACG
GGCACCTACAGCACCGCGACAGCCACCTCCTGGCCCAGCACGCTGACCAGCGGCTCGCCAGGCAGCACCA
CCACCGTGGGCACCACGACCAGTACCACCTCTGGCACCGCCGCCGAGACCGCCTGTGCGACCCCTACCGC
CGTGGCCGTCACCTTTAACGAGATCGCCACCACCACCTACGGCGAGAATGTTTACATTGTTGGGTCCATC
TCCGAGCTCGGGAACTGGGATACCAGCAAAGCAGTGGCCCTGAGTGCGTCCAAGTATACCTCCAGCAATA
ACCTCTGGTACGTGTCCGTCACCCTGCCGGCTGGCACGACATTCGAGTACAAGTATATCCGCAAGGAAAG
CGATGGCTCGATCGTGTGGGAGAGTGACCCCAACCGCTCGTATACGGTGCCGGCAGCTTGTGGAGTGTCT
ACTGCGACCGAGAATGATACTTGGCAGTGA
SEQ ID NO: 9-AfGA1
GCGGCGGCCGCACCATGCCTCGCCTTTCCTACGC
SEQ ID NO: 10-AfGA1
CCGGCGCGCCCTTATCACTGCCAAGTATCATTCTCG
SEQ ID NO: 11-AfGA1 and AfGA2 signal peptide
MPRLSYALCALSLGHAAIA
SEQ ID NO: 12-AfGA1 Mature form
APQLSARATGSLDSWLGTETTVALNGILANIGADGAYAKSAKPGIIIASPSTSEPDYYYTWTRDAALVTK
VLVDLFRNGNLGLQKVITEYVNSQAYLQTVSNPSGGLASGGLAEPKYNVDMTAFTGAWGRPQRDGPALRA
TALIDFGNWLIDNGYSSYAVNNIWPIVRNDLSYVSQYWSQSGFDLWEEVNSMSFFTVAVQHRALVEGSTF
AKRVGASCSWCDSQAPQILCYMQSFWTGSYINANTGGGRSGKDANTVLASIHTFDPEAGCDDTTFQPCSP
RALANHKVYTDSFRSVYAINSGIPQGAAVSAGRYPEDVYYNGNPWFLTTLAAAEQLYDAIYQWKKIGSIS
ITSTSLAFFKDIYSSAAVGTYASSTSTFTDIINAVKTYADGYVSIVQAHAMNNGSLSEQFDKSSGLSLSA
RDLTWSYAAFLTANMRRNGVVPAPWGAASANSVPSSCSMGSATGTYSTATATSWPSTLTSGSPGSTTTVG
TTTSTTSGTAAETACATPTAVAVTFNEIATTTYGENVYIVGSISELGNWDTSKAVALSASKYTSSNNLWY
VSVTLPAGTTFEYKYIRKESDGSIVWESDPNRSYTVPAACGVSTATENDTWQ
SEQ ID NO: 13-AfGA2 Mature form
APQLSARATGSLDSWLGTETTVALNGILANIGADGAYAKSAKPGIIIASPSTSEPDYYYTWTRDAALVTK
VLVDLFRNGNLGLQKVITEYVNSQAYLQTVSNPSGGLASGGLAEPKYNVDMTAFTGAWGRPQRDGPALRA
TALIDFGNWLIDNGYSSYAVNNIWPIVRNDLSYVSQYWSQSGFDLWEEVNSMSFFTVAVQHRALVEGSTF
AKRVGASCSWCDSQAPQILCYMQSFWTGSYINANTGGGRSGKDANTVLASIHTFDPEAGCDDTTFQPCSP
RALANHKVYTDSFRSVYAINSGIPQGAAVSAGRYPEDVYYNGNPWFLTTLAAAEQLYDAIYQWKKIGSIS
ITSTSLAFFKDIYSSAAVGTYASSTSTFTDIINAVKTYADGYVSIVQAHAMNNGSLSEQFDKSSGLSLSA
RDLTWSYAAFLTANMRRNGVVPAPWGAASANSVPSSCSMGSATGTYSTATATSWPSTLTSGSPGSTTTVG
TTTSTTSGTATETACATPTAVAVTFNEIATTTYGENVYIVGSISELGNWDTSKAVALSASKYTSSNNLWY
VSVTLPAGTTFEYKYIRKESDGSIVWESDPNRSYTVPAACGVSTATENDTWR
SEQ ID NO: 14-AfGA2 gene of pTrex3gM-AfGA2
ATGCCTCGACTGAGCTACGCTCTCTGCGCTCTGTCCCTGGGTCACGCTGCCATCGCCGCTCCCCAACTGA
GCGCCCGAGCTACTGGCAGCCTCGATTCCTGGCTGGGCACTGAGACCACCGTTGCTCTGAACGGCATCCT
CGCTAACATCGGCGCTGATGGTGCCTATGCCAAGAGCGCTAAACCTGGCATCATCATCGCCAGCCCTAGC
ACCAGCGAGCCTGATTACTACTATACTTGGACCCGCGACGCTGCTCTGGTCACCAAGGTCCTCGTTGACC
TGTTCCGCAATGGTAACCTGGGCCTCCAGAAAGTCATTACCGAGTACGTCAACAGCCAAGCTTATCTGCA
AACCGTTAGCAATCCCTCCGGTGGCCTCGCTTCCGGCGGCCTGGCCGAGCCCAAATACAACGTCGACATG
ACCGCCTTTACCGGTGCCTGGGGTCGCCCCCAGCGAGATGGCCCTGCCCTGCGCGCCACCGCTCTCATCG
ACTTCGGCAACTGGCTGATCGACAACGGCTATTCCAGCTATGCTGTCAACAACATTTGGCCCATCGTCCG
CAACGACCTGTCCTATGTTTCCCAATACTGGTCCCAGTCCGGTTTCGACCTCTGGGAGGAGGTTAATTCC
ATGAGCTTTTTCACCGTCGCTGTCCAACATCGAGCTCTCGTCGAGGGCTCCACTTTCGCTAAGCGCGTCG
GCGCCAGCTGTTCCTGGTGCGATTCCCAGGCCCCTCAGATTCTGTGCTACATGCAGTCCTTTTGGACCGG
TAGCTATATCAATGCCAATACCGGCGGTGGTCGAAGCGGCAAGGACGCTAATACTGTTCTGGCTTCCATC
CACACCTTCGATCCCGAGGCCGGCTGTGATGATACTACCTTTCAGCCCTGCTCCCCTCGCGCTCTCGCCA
ACCATAAAGTTTACACCGACAGCTTTCGCAGCGTTTACGCCATCAACTCCGGCATTCCTCAAGGCGCTGC
TGTTTCCGCTGGTCGCTACCCCGAGGACGTTTACTATAATGGCAACCCCTGGTTCCTCACTACTCTGGCT
GCTGCTGAGCAGCTCTATGACGCTATCTACCAATGGAAGAAAATCGGCAGCATCAGCATTACTTCCACCT
CCCTCGCCTTCTTCAAAGACATCTATAGCTCCGCTGCCGTTGGCACTTATGCTTCCTCCACTAGCACTTT
CACTGATATTATCAACGCTGTTAAAACCTACGCTGACGGCTACGTCAGCATCGTTCAAGCCCACGCTATG
AACAACGGTTCCCTCTCCGAGCAGTTCGACAAGTCCAGCGGTCTGAGCCTCAGCGCTCGCGACCTCACCT
GGTCCTACGCCGCCTTCCTGACTGCCAACATGCGCCGAAACGGCGTCGTTCCTGCCCCTTGGGGTGCCGC
CAGCGCCAATTCCGTCCCCAGCAGCTGTAGCATGGGCTCCGCCACTGGTACCTACAGCACCGCTACCGCT
ACTAGCTGGCCCAGCACCCTGACTAGCGGCTCCCCCGGTTCCACTACTACCGTCGGCACCACTACCTCCA
CCACTTCCGGTACTGCCACCGAGACTGCCTGTGCCACCCCTACCGCCGTCGCCGTCACCTTTAACGAGAT
TGCTACCACCACCTACGGCGAGAACGTCTACATCGTCGGTAGCATCTCCGAGCTCGGCAATTGGGACACT
TCCAAGGCTGTCGCCCTGTCCGCCTCCAAATATACTAGCAGCAACAACCTGTGGTATGTCTCCGTTACCC
TGCCTGCTGGTACTACTTTTGAGTACAAGTACATTCGCAAAGAGTCCGATGGCTCCATCGTTTGGGAGTC
CGATCCCAACCGAAGCTACACCGTTCCCGCTGCTTGTGGCGTCTCCACTGCTACTGAGAATGACACCTGG
CGCTAA

1. A method of simultaneously liquefying and saccharifying a composition comprising starch to produce a composition comprising glucose, wherein said method comprises:
(i) contacting said composition comprising starch with an isolated AfGATR, or variant thereof, having at least 80% sequence identity to SEQ ID NO: 12 or 13 and an α-amylase; and
(ii) liquefying and saccharifying the composition comprising starch above the gelatinization temperature of said starch in the same reaction vessel to produce said glucose composition; wherein said AfGATR, or variant thereof, and said α-amylase catalyze the saccharification of the composition comprising starch to the composition comprising glucose.

2. The method of claim 1, wherein said AfGATR, or variant thereof, has at least 70% activity at 74° C. at pH 5.0 over 10 min.

3. The method of claim 2, wherein said AfGATR, or variant thereof, is AfGA1TR, or variant thereof.

4. The method of claim 3, wherein said AfGA1TR, or variant thereof, has at least 70% activity over a temperature range of 55°-74° C. at pH 5.0 over 10 min.

5. The method of claim 4, wherein said AfGA1TR, or variant thereof, has an optimum temperature of about 68° C.

6. The method of claim 2, wherein said AfGATR, or variant thereof, is AfGA2TR, or variant thereof.

7. The method of claim 6, wherein said AfGA2TR, or variant thereof, has at least 70% activity over a temperature range of 61° to 74° C. at pH 5.0 over 10 min.

8. The method of claim 7, wherein said AfGA2TR, or variant thereof, has an optimum temperature of about 69° C.

9. The method of claim 1, wherein said AfGATR, or variant thereof, comprises an amino acid sequence with at least 90%, 95%, or 99% amino acid sequence identity to SEQ ID NO: 12.

10. The method of claim 9, wherein said AfGATR, or variant thereof, comprises SEQ ID NO: 12.

11. The method of claim 1, wherein said AfGATR, or variant thereof, consists of an amino acid sequence with at least 80%, 90%, 95%, or 99% amino acid sequence identity to SEQ ID NO: 12.

12. (canceled)

13. The method of claim 1, wherein said AfGATR, or variant thereof, comprises an amino acid sequence with at least 90%, 95%, or 99% amino acid sequence identity to SEQ ID NO: 13.

14. The method of claim 13, wherein said AfGATR, or variant thereof, comprises SEQ ID NO: 13.

15. The method of claim 1, wherein said AfGATR, or variant thereof, consists of an amino acid sequence with at least 80%, 90%, 95%, or 99% amino acid sequence identity to SEQ ID NO: 13.

16. (canceled)

17. The method of claim 1, wherein at least 93% of the starch is solubilized after about 23 hrs.

18-20. (canceled)

21. The method of claim 1, further comprising adding a pullulanase.

22-24. (canceled)

25. The method of claim 1, wherein said liquefaction and saccharification is conducted between about 70° C. to about 80° C.

26. The method of claim 25, wherein said liquefaction and saccharification is conducted at about 70° C.

27. The method of claim 25, wherein said liquefaction and saccharification is conducted at about 75° C.

28. The method of claim 25, wherein said liquefaction and saccharification is conducted at about 80° C.

29. The method of claim 1, wherein said liquefaction and saccharification is conducted over a pH range of pH 3.0-pH 7.5.

30. The method of claim 29, wherein said pH range is pH 3.5-pH 7.0.

31. The method of claim 29, wherein said pH range is pH 4.0-pH 6.7.

32. The method of claim 1, wherein said liquefaction and saccharification is conducted for at least 23 hours, at about pH 4.0-6.7 and 34% DS, and at a temperature of about 70° C. to 84° C.

33. The method of claim 32, wherein said liquefaction and saccharification is conducted for at least about 23 hours, at about pH 5.0 and at a temperature of about 70° C. to about 80° C.

34. The method of claim 1, further comprising fermenting the glucose composition to produce an End of Fermentation (EOF) product.

35. The method of claim 34, wherein the EOF product comprises a metabolite.

36. The method of claim 35, wherein the metabolite is citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, an amino acid, lysine, itaconic acid, 1,3-propanediol, or isoprene.

37. The method of claim 1, further comprising adding a glucoamylase that is not AfGATR, or variant thereof, hexinase, xylase, glucose isomerase, xylose isomerase, phosphatase, phytase, pullulanase, β-amylase, an additional α-amylase, protease, cellulase, hemicellulase, lipase, cutinase, isoamylase, redox enzyme, esterase, transferase, pectinase, alpha-glucosidase, beta-glucosidase, or a combination thereof, to said starch solution.

38. The method of claim 1, wherein said isolated AfGATR, or variant thereof, is secreted by a Trichoderma reesei host cell.

39. The method of claim 38, wherein the host cell further expresses and secretes an α-amylase and/or a pullulanase.

40-49. (canceled)
